(19) United States
(12) Patent Application Publication
(10) Pub. No.: US 2013/0178454 A1
(43) Pub. Date: Jul. 11, 2013

(54) COMBINATION OF TESTOSTERONE AND ORNITHINE DECARBOXYLASE (ODC) INHIBITORS

(71) Applicants: Shalender Bhasin, Weston, MA (US); Ravi Jasuja, Weston, MA (US)

(72) Inventors: Shalender Bhasin, Weston, MA (US); Ravi Jasuja, Weston, MA (US)

(21) Appl. No.: 13/679,889

(22) Filed: Nov. 16, 2012

Related U.S. Application Data
(60) Provisional application No. 61/560,870, filed on Nov. 17, 2011.

(51) Int. Cl. A61K 31/568; A61K 45/06; A61K 31/197; A61K 45/00

(52) U.S. Cl. CPC A61K 31/568; A61K 45/00; A61K 45/06; A61K 31/197
USPC 514/171

(57) ABSTRACT

The present invention is generally directed to a method of increasing testosterone levels in a subject comprising; administration of testosterone or analogue thereof along with an ornithine decarboxylase (ODC) inhibitor. The method selectively promotes beneficial effects of testosterone, while preventing side effects associated with testosterone administration. The present invention also directed to pharmaceutical composition and kits comprising testosterone or analogue thereof; ornithine decarboxylase inhibitors. The composition selectively promotes beneficial effects of testosterone, while preventing side effects of testosterone administration. In some embodiments, the present invention relates to a method of treatment of low testosterone levels in a subject by administrating testosterone and ODC inhibitor while preventing side effects associated with testosterone administration. The method and composition of the invention particularly prevents potential adverse effects on the prostate in men and virilization in women.

### CROSS REFERENCE TO RELATED APPLICATIONS

This application claims the benefit of priority to U.S. provisional application No. 61/560,870 filed on 17 Nov. 2011, which is hereby expressly incorporated by reference in its entirety.

### GOVERNMENT SUPPORT

This invention was made with U.S. Government Support under Contract Number 5P30AG031679 awarded by the National Institutes of Health (NIH). The Government has certain rights in the invention.

### FIELD OF THE INVENTION

The present invention is generally directed to a method of increasing testosterone levels in a subject comprising; administration of testosterone or analogue thereof along with an ornithine decarboxylase (ODC) inhibitor. The method selectively promotes beneficial effects of testosterone, while preventing side effects associated with testosterone administration. The present invention also directed to pharmaceutical composition and kits comprising testosterone or analogue thereof; ornithine decarboxylase inhibitors. The composition selectively promotes beneficial effects of testosterone, while preventing side effects of testosterone administration. The method and composition of the invention particularly prevents potential adverse effects on the prostate in men and virilization in women.

### BACKGROUND OF THE INVENTION

Testosterone (T) is a primary androgenic hormone produced predominantly in the interstitial cells of the testes and is responsible for normal growth, development and maintenance of male sex organs and secondary sex characteristics (e.g., deepening voice, muscular development, facial hair, etc.). Throughout adult life, testosterone is necessary for proper functioning of the testes and its accessory structures, prostate and seminal vesicle; for sense of well-being; and for maintenance of libido, erectile potency.

Testosterone deficiency is insufficient secretion of Testosterone characterized by low serum Testosterone concentrations, can give rise to medical conditions (e.g., hypogonadism) in males. Symptoms associated with male hypogonadism include impotence and decreased sexual desire, fatigue and loss of energy, mood depression, regression of secondary sexual characteristics, decreased muscle mass, and increased fat mass. Furthermore, hypogonadism in men is a risk factor for anemia, osteoporosis, metabolic syndrome, type II diabetes and cardiovascular disease.

Various testosterone replacement therapies are commercially available for the treatment of male hypogonadism. Pharmaceutical preparations include both testosterone and testosterone derivatives in the form of intramuscular injections, implants, oral tablets of alkylated Testosterone (e.g., methyltestosterone), topical gels, or topical patches. However, all of the current T therapies have some adverse side effects, such as erythrocytosis, liver toxicity, prostate hyperplasia, and prostate-side effects. Over time, therefore, the current methods of treating testosterone deficiency suffer from poor compliance and thus unsatisfactory treatment of men with low Testosterone.

As men age, they generally produce less testosterone from the Leydig cells in their testes. Middle-aged men possessing the lowest quartile of testosterone levels (0.003 to 0.23 nmol/liter) are more likely to suffer from type 2 diabetes, metabolic disease, dyslipidemia, and obesity. Additionally, patients suffering from AIDS experience low testosterone or low androgen levels that result in a reduction of muscle and bone mass.

Testosterone replacement therapy helps to provide and maintain normal levels of testosterone. The market for this therapy, however, is far from saturated, with only 5% of the people affected by this disorder receiving treatment.

Testosterone replacement (e.g., administering exogenous testosterone) is often associated with undesired consequences and side effects in men and women like prostate associated side effects and virilization respectively. Accordingly, alternative ways/methods of testosterone administration are needed which selectively promotes beneficial effects of testosterone, while preventing side effects associated with testosterone administration. Additionally alternate methods and pharmaceutical compositions are needed that effectively treat low testosterone symptoms in male subjects while preventing associated side effects and undesired consequences.

Testosterone is a primary androgen which is largely produced by Leydig cells in the testes. Production increases in response to luteinizing hormone (LH). Longitudinal studies of aging men clearly demonstrate age-dependent reductions in both total and free testosterone levels. Further studies reveal association between low testosterone levels with type 2 diabetes or both metabolic syndrome and diabetes. Other cross-sectional studies reveal a correlation between low levels of both free and total testosterone and dyslipidemia, obesity, and insulin resistance or hyperinsulinemia. The present invention is directed toward overcoming one or more of the problems discussed above.

Testosterone maintains a woman's general wellbeing, mood and sexual functioning. Besides its psychological and sexual effects, adequate levels of testosterone play an important role in helping women maintain a healthy body composition. Obese women are given low doses of synthetic analogues of testosterone (nandralone). Women have been shown to loose more body fat and subcutaneous abdominal fat, and gain muscle mass by testosterone administration.

However, high testosterone levels in women also causes significant side effects like virilization. Virilization is an indication of significantly high levels of testosterone in women. Symptoms of virilization include enlargement of the clitoris, lowering of the voice, breast atrophy and other side effects of high testosterone such as hirsutism. This condition is often associated with tumors of the adrenal glands or ovaries. Virilization also includes increased libido and male pattern muscle mass gain and if left untreated, can lead to increased risks of heart disease and hypertension.

Accordingly, alternative ways/methods of testosterone administration are needed which selectively promotes beneficial effects of testosterone, while preventing side effects associated with testosterone administration. There is also thus clearly a need for novel therapies and methods that overcome the decrease in testosterone level in a male and female subject but at the same time reduce the undesired consequences and side effects associated with high testosterone levels.

Additionally alternate methods and pharmaceutical compositions are needed that effectively treat low testosterone symptoms in male and female subjects while preventing associated side effects and undesired consequences.

### SUMMARY OF THE INVENTION

An object of the present invention is to provide alternative ways/methods of testosterone administration which selectively promotes beneficial effects of testosterone, while preventing side effects associated with testosterone administration.

An object of the present invention is to also to provide compositions comprising testosterone or analogue thereof which selectively promotes beneficial effects of testosterone, while preventing side effects associated with testosterone administration.

Another object of the present invention is to provide a method for increasing testosterone levels in a subject comprising administration of; testosterone or analogue thereof; and an ornithine decarboxylase (ODC) inhibitor. The method of the invention selectively promotes beneficial effects of testosterone, while preventing side effects related to testosterone administration. Another object of the present invention is to s provide to a subject in need with selective beneficial effect of testosterone, or an analogue thereof, while reducing adverse side effects of testosterone like prostate related side effects in males and virilization in females.

Yet another object of the present invention is to provide a method for increasing testosterone levels in a subject, such that said method selectively promotes beneficial effects of testosterone, while preventing side effects related to testosterone administration.

Yet another object of the present invention is to provide a method for treating a subject with low testosterone levels by selectively promoting beneficial effects of testosterone, while preventing side effects related to testosterone administration, comprising administration of; testosterone or analogue thereof; and an ornithine decarboxylase (ODC) inhibitor.

Yet another object of the present invention is to provide a method for treating hypogonadism in males by selectively promoting beneficial effects of testosterone, while preventing prostate related side effects, comprising administration of; testosterone or analogue thereof; and an ornithine decarboxylase (ODC) inhibitor.

A yet another objective of the present invention is to provide a kit comprising a combination of testosterone or analogue thereof; and an ornithine decarboxylase (ODC) inhibitor.

There are several adverse side effects associated with testosterone therapy, including but not limited to, prostate related side effects in men and virirlization in women. The side effects of testosterone in particular also include increased incidence of prostate cancer, prostate growth, increased red cell mass, cardiac adverse side-effects and the like. In particular, a side-effect of current testosterone treatments, e.g., testosterone therapy, are commonly associated with the prostate. In particular, testosterone therapy can lead to prostate growth, which can have a negative effect on men in two ways. Firstly, the prostate may increase in size (benign prostatic hyperplasia (BPH), which may cause problems with urination. Second, there is concern that testosterone therapy may also lead to the growth of subclinical prostate cancers that are commonly present in many middle aged and older men. Furthermore, prostate cancer is a common cancer for older men, and is the second most common cause of cancer deaths in older men. Therefore, due to the prostate-associated side effects of testosterone, testosterone therapy in older men or men with prostate cancer or with high levels of PSA (prostate specific antigen) is not recommended long term. An object, therefore of the present invention is to provide alternative ways/methods of testosterone administration which selectively promotes beneficial effects of testosterone, while preventing its adverse side effects.

It is well known in the art that ODC is required and mediates testosterone's growth promoting effect on muscle and prostate. (see Lee et al., Am J. Phys Endrocrinol Metab 2011; 301: E172-179 “Ornithine decarboxylase is upregulated by the androgen receptor in skeletal muscle and regulates myoblast proliferation”; Kapyaho et al (Biochem J., 1984; 219; 811-817); Danzin et al. (Biochem. J. (1982) 202 (175-181)); “Effect on prostatic growth of 2-difluoromethylomithine, an effective inhibitor of ornithine decarboxylase” and Lee et al., J. Cell Physiol, 2011; 226: 1453-60; Polyamines, androgens and skeletal muscle hypertrophy). These reports indicated that ODC mediates muscle and prostate growth by testosterone. Having said this, a person skilled in the art would be directed to understand that ODC is obligatory for muscle growth and therefore ODC inhibition will prevent muscle growth by testosterone. In direct contrast, the inventors demonstrate that ODC activity is not essential for the anabolic effects of testosterone in muscles, but is essential for the effects of testosterone in the prostate and that administration of testosterone and ODC inhibitor promotes muscle growth without stimulating prostate, thereby marking a breakthrough in the field and teaching away from the prior art. Accordingly, the inventors have discovered that by inhibiting ODC activity, the effects of testosterone on the prostate can be excluded while maintaining the beneficial effects on muscle growth and other tissues.

The inventors have discovered that testosterone's effects on the prostate require an essential mediation of the ornithine decarboxylase (ODC) pathway. The inventors have demonstrated that inhibition of ODC in the presence of testosterone blocks testosterone's effects on the prostate without affecting testosterone's other beneficial effects, e.g., increasing muscle mass, and/or for the treatment of low testosterone disorders and androgen deficiency. Accordingly, the inventors have demonstrated that they can increase the selectivity of testosterone and increase the selective beneficial effects of testosterone in a subject without adverse prostate-associated side effects by administering testosterone or an analogue or pharmaceutically acceptable salt in combination with an ODC inhibitor.

Testosterone and follistatin (an endogenous protein that increases muscle mass) are both known to increase muscle mass. The inventors herein have surprisingly discovered that follistatin has no effect on the prostate in vitro and in vivo. Using microarrays, the inventors have characterized the signaling pathways that are differentially activated by testosterone and follistatin in the muscle and in the prostate and have discovered that testosterone activates ornithine decarboxylase (ODC) and polyamine pathway in the prostate, whereas follistatin surprisingly does not activate prostate ornithine decarboxylase (ODC) and polyamine pathway in the prostate. Also, the inventors have found that testosterone stimulates the expression of follistatin which is associated with increased skeletal muscle mass. Thus, the inventors have demonstrated that testosterone's effects on the prostate can be blocked by an ODC inhibitor, such as, but not limited to 2-difluoromethylornithine (DFMO), while DFMO does not affect testosterone's beneficial effects on the muscle. In other words, it has been found that testosterone when administered along with ODC inhibitors inhibits prostate growth while simultaneously increasing muscle mass. In some embodiments, an ornithine decarboxylase inhibitor is N-(4′-Pyridoxyl)-Ornithine(BOC)-OMe [POB]. In some embodiments, an ornithine decarboxylase inhibitor is a-methyl ornithine. In some embodiments, an ornithine decarboxylase inhibitor is antizyme (AZ), which binds to ODC and accelerates the ATP-dependent degradation of the ODC enzyme, as disclosed in U.S. Pat. No. 6,914,079, which is incorporated herein in its entirety by reference (and Chattopadhyay et al., 2001, JBC, 276; 21235-21241 “Antizyme Regulates the Degradation of Ornithine Decarboxylase in Gission Yeast Schizosaccharomyces pombe”.

Thus, the inventors have demonstrated a method to selectively target testosterone's anabolic effects on the muscle and reduce prostate-associated side effects by administering testosterone in combination with an ODC inhibitor, such as DFMO. Thus, combination of testosterone plus an ODC inhibitor, such as DFMO can serve as a selective anabolic agent that can increase muscle mass, strength and physical function while sparing the prostate. Thus, one aspect of the present invention relates to the use of combination of testosterone plus an ODC inhibitor, such as DFMO in a new formulation for testosterone replacement therapy for conditions in which testosterone may be contraindicated or associated with high risk of adverse prostate events, such as men with prostate cancer who have undergone radical prostatectomy, hypogonadal men at risk of prostate cancer, or older men with low testosterone level. Testosterone and an ODC inhibitor, e.g., DFMO are each approved for other indications, and are available commercially.

Accordingly, a further aspect of the present invention relates to the selective beneficial effect of testosterone, or an analogue thereof particularly while reducing adverse side effects associated with administering testosterone by using testosterone in combination with an ODC inhibitor.

The present invention also relates to a method of treating a low-testosterone associated disorder in a subject while preventing side effects related to testosterone administration, like an increase in prostate mass or prostate growth in the subject by at least 10%.

Accordingly, one aspect of the present invention relates to a method of treating a subject with a low testosterone disease or disorder by administering a composition comprising a combination of testosterone or an analogue thereof and an ornithine decarboxylase (ODC) inhibitor as disclosed herein. In some embodiments, the ODC inhibitor is 2-difluoromethylornithine (DFMO) or an analogue or derivative thereof. In some embodiments, the subject is a human. Typically, a subject is a male subject, however, female subjects with low androgen levels are also amenable to treatment according to the methods as disclosed herein. Additional aspects of the present invention also relate to methods to increase testosterone production in a subject, for example, a subject with low testosterone levels. Other aspects of the present invention relate to a method for the treatment of a subject with a disease or disorder associated with low testosterone, for example, but not limited to, a hypogonadal subject, or a subject with male menopause, or a subject who has undergone removal of part, or a whole testis for example, after testicular cancer, or where the subject has previously, or is undergoing radiation therapy for testicular cancer, or a subject has one or more symptoms of a low-testosterone associated disease such as, but not limited to; HIV-infection, end stage renal disease, type 2 diabetes, metabolic syndrome, dyslipidemia, obesity, insulin resistance or hyperinsulinemia. Another aspect of the present invention relates to kits comprising the compositions as disclosed herein, for example, for use in methods for the treatment of low testosterone diseases and disorders, and associated diseases and disorders. In some embodiments, the kit further comprises instructions for use.

Accordingly, the present invention provides co-administrable combination of testosterone or an analogue thereof and an ornithine decarboxylase inhibitor, that allows for the beneficial sexual and anabolic effects of testosterone like improvement in muscle mass and muscle performance, and physical function or measures of physical function in subjects with sarcopenia, frailty, or falls, or increased risk of falls with diminished side effects connected with testosterone administration. The present invention also provides a method for increasing testosterone levels in a subject comprising administration of; testosterone or analogue thereof; and an ornithine decarboxylase (ODC) inhibitor or salts thereof. The method selectively promotes beneficial effects of testosterone, while preventing side effects related to testosterone administration. The present invention further provides a pharmaceutical composition comprising testosterone or analogue thereof; ornithine decarboxylase inhibitors. Said composition selectively promotes beneficial effects of testosterone, while preventing side effects related to testosterone administration.

Yet another aspect of the present invention relates to a method of treating a subject with a low testosterone disease or disorder by administering a composition comprising a combination of testosterone or an analogue thereof and an ornithine decarboxylase (ODC) inhibitor. By administering to the subject a testosterone or analogue thereof in first therapeutically effective amount and an ornithine decarboxylase (ODC) inhibitor in a second therapeutically effective amount, side effects related to administration of testosterone are reduced by 10%.

In some embodiments, the effective amount of the ODC inhibitor in the presence of testosterone can be assessed in a rodent (e.g., mouse) model, e.g., a therapeutically effective amount of an ODC inhibitor is the amount which in the presence of testosterone, inhibits the testosterone-induced increase in prostate growth in the mouse by at least 10% as compared to the amount of prostate growth occurring in the presence of testosterone alone (e.g. in the absence of the ODC inhibitor).

In some embodiments, the effective amount of the ODC inhibitor in the presence of testosterone can be assessed in an animal (e.g., mouse) model, e.g., a therapeutically effective amount of an ODC inhibitor is the amount which, in the presence of testosterone, inhibits the testosterone-induced increase in prostate mass in the mouse by at least 10% as compared to the amount of prostate growth occurring in the presence of testosterone alone (e.g. in the absence of the ODC inhibitor).

In some embodiments, an effective amount of an ODC inhibitor can inhibit the testosterone-induced increase in prostate mass and/or prostate growth in subject or a mouse model by at least about 10%, or at least about 20%, or at least about 30%, or at least about 40%, or at least about 50%, or at least about 60%, or at least about 70%, or at least about 80%, or at least about 90%, or more than 90%, as compared to the amount of prostate mass and/or prostate growth occurring in the presence of testosterone alone (e.g. in the absence of the ODC inhibitor). In some embodiments, an effective amount of an ODC inhibitor can inhibit the testosterone-induced increase in prostate mass and/or prostate growth in subject or a mouse model by about 90% to 100% as compared to the amount of prostate mass and/or prostate growth occurring in the presence of testosterone alone (e.g. in the absence of the ODC inhibitor).

Stated in another way and for the purposes of illustration only, if, for example, testosterone alone (in the absence of the ODC inhibitor) causes an increase in prostate mass and/or prostate growth in subject or mouse model by 10% as compared to absence of testosterone, an effective amount of an ODC inhibitor can prevent this 10% increase by at least 10%, thus amount of prostate growth and/or mass would be 9% or less than 9% if testosterone is administered in combination or conjunction with an ODC inhibitor. As another illustrative example, if testosterone alone causes a 10% increase in prostate mass and/or prostate growth in a mouse model or a subject, an effective amount of an ODC inhibitor which prevents this 10% increase by at least 90% will result in an increase in prostate growth and/or prostate mass by less than 1% if testosterone is administered in combination or conjunction with an ODC inhibitor.

Aspects of the invention concern a pharmaceutical composition for selectively promoting beneficial effects of testosterone, while preventing side effects related to testosterone administration comprising testosterone or analogue thereof; ornithine decarboxylase inhibitors. In some embodiments, the pharmaceutical composition has testosterone analogues that are selected from the group consisting of testosterone ester, testosterone salts, testosterone prodrugs, fatty acid ester of testosterone, or testosterone metabolites. In other embodiments, the pharmaceutical composition has a testosterone metabolite that is dihydrotestosterone. In other embodiments, the pharmaceutical composition includes an ester of testosterone that is a C2-C13 alkyl ester (e.g., an undecanoate acid ester of testosterone). In more embodiments, the pharmaceutical composition has an ornithine decarboxylase inhibitor that is selected from the group consisting of 2-difluoromethylomithine, N-(4′-Pyridoxyl)-Ornithine(BOC)-OMe(POB), α-methyl ornithine, 1,4-diamino-2-butanone (DAB), and antizyme (AZ) or combination thereof. These pharmaceutical compositions can be provided in a dosage of 0.002 mg/kg to about 50 mg/kg per day each of testosterone or analogue thereof and ODC inhibitor to a subject. In some embodiments, the side effects that are prevented comprise prostate related side effects in males and virilization in females and, in some embodiments, the side effects related to testosterone administration are prevented by at least 10%. In more embodiments, the pharmaceutical composition described above, provides therapeutic benefit while reducing prostate related side effects such as prostate cancer and enlarged prostate or hirsutism and/or acne. In other embodiments, the pharmaceutical composition described above, provides beneficial effects such as improvement in muscle mass and muscle performance, and physical function or measures of physical function in subjects with sarcopenia, frailty, or falls, or increased risk of falls. In other embodiments, the pharmaceutical composition described above, provides beneficial effects such as improved mobility in subjects with mobility limitation associated with sarcopenia, muscle atrophy and weakness due to acute or chronic illness, bed rest, or fracture. In other embodiments, the pharmaceutical composition described above, provides beneficial effects such as treating muscle wasting and physical dysfunction in subjects who have muscle wasting due to bed rest, immobilization, surgery, or placement of an orthopedic cast or because of any acute or chronic illness and to promote recovery of physical function and strength and promote mobility after an acute or chronic illness, or hip fracture. In other embodiments, the pharmaceutical composition described above, is provided or formulated in a timed release, sustained release or controlled release composition. In more embodiments, said pharmaceutical composition further comprises an additional agent (e.g., leutropin, human chorionicgonadotrophin, anti-cancer agent, or an anti-viral agent. The pharmaceutical composition described above, can be administered in the form of gel, tablet, capsule, granulate, food product, troches, dispersions, suspensions solutions, implants, or patches.

Additional embodiments include a co-administrable combination of testosterone or analogue thereof in a first therapeutically effective amount, and an ornithine decarboxylase (ODC) inhibitor in a second therapeutically effective amount for selectively promoting beneficial effects of testosterone, while preventing side effects related to testosterone administration. This co-administrable combination can include a testosterone analogue selected from the group consisting of testosterone ester, testosterone salts, testosterone prodrugs, fatty acid ester of testosterone, and testosterone metabolites. In some embodiments, The co-administrable combination comprises the testosterone metabolite dihydrotestosterone. In other embodiments, the co-administrable combination comprises an ester of testosterone that is a C2-C13 alkyl ester. In other embodiments, the co-administrable combination comprises an undecanoate acid ester of testosterone. In other embodiments, the co-administrable combination comprises an ornithine decarboxylase inhibitor that is selected from the group consisting of 2-difluoromethylornithine, N-(4′-Pyridoxyl)-Ornithine(BOC)-OMe(POB), α-methyl ornithine, 1,4-diamino-2-butanone (DAB), and antizyme (AZ) or a combination thereof. In other embodiments, the co-administrable combination prevents side effects such as prostate related side effects in males and virilization in females. In other embodiments, the co-administrable combination selectively promotes beneficial effects of testosterone, while preventing side effects related to testosterone administration by at least 10%. In other embodiments, the co-administrable combination prevents side effects such as prostate cancer and enlarged prostate or hirsutism or acne. In other embodiments, the co-administrable combination provides beneficial effects such as improvement in muscle mass and muscle performance, and physical function or measures of physical function in subjects with sarcopenia, frailty, or falls, or increased risk of falls. In other embodiments, the co-administrable combination provides benefits such as improved mobility in subjects with mobility limitation associated with sarcopenia, muscle atrophy and weakness due to acute or chronic illness, bed rest, or fracture. In other embodiments, the co-administrable combination provides benefits such as treating muscle wasting and physical dysfunction in subjects who have muscle wasting due to bed rest, immobilization, surgery, or placement of an orthopedic cast or because of any acute or chronic illness, and promoting recovery of physical function and strength, and mobility after an acute or chronic illness, or hip fracture.

Additional embodiments include methods for increasing testosterone levels in a subject, for selectively promoting beneficial effects of testosterone, while preventing side effects related to testosterone administration, comprising administration of; testosterone or analogue thereof; and an ornithine decarboxylase (ODC) inhibitor. In some embodiments, the subject has low testosterone levels (e.g., a testosterone level of less than 350 pg/ml), which can be due to age-related low testosterone production and/or a low-testosterone associated disorder such as type 2 diabetes, metabolic syndrome, dyslipidemia, obesity-related insulin resistance or hyperinsulinemia, male menopause, andropause, hypogonadotropichypogonadism, testicular failure, hyperprolactinemia, hypopituitarism, hypothalamic or pituitary disease, genetic diseases such as Klinefelter's syndrome, Kallman's syndrome, and Prader-Willi syndrome, infertility, myotonic dystrophy, acquired damage to the testes, such as alcoholism, physical injury, or viral diseases like mumps or testicular cancer. In some embodiments, the method is practiced by providing an ornithine decarboxylase inhibitor that is selected from the group consisting of 2-difluoromethylornithine, N-(4′-Pyridoxyl)-Ornithine(BOC)-OMe(POB), α-methyl ornithine, 1,4-diamino-2-butanone (DAB), and antizyme (AZ) or combination thereof. In other such methods, a testosterone analogue is used and this analogue is selected from the group comprising of testosterone ester, testosterone salts, testosterone prodrugs, fatty acid ester of testosterone, and testosterone metabolites. In some embodiments, said testosterone metabolite is dihydrotestosterone, or said ester of testosterone is a C2-C13 alkyl ester or said ester of testosterone is an undecanoate acid ester of testosterone. In some embodiments, the testosterone or analogue thereof and ODC inhibitor are each administered in an amount of about 0.0002 mg/kg to about 50 mg/kg of body weight per day. In other embodiments, the method provides benefits such as reducing the side effects associated with testosterone therapy including prostate related side effects in males and virilization in females. In other embodiments, the method provides benefits such as improvement in muscle mass and muscle performance, and physical function or measures of physical function in subjects with sarcopenia, frailty, or falls, or increased risk of falls. In other embodiments, the method provides benefits such as improved mobility in subjects with mobility limitation associated with sarcopenia, muscle atrophy and weakness due to acute or chronic illness, bed rest, or fracture. In other embodiments, the method provides benefits such as treating muscle wasting and physical dysfunction in subjects who have muscle wasting due to bed rest, immobilization, surgery, or placement of an orthopedic cast or because of any acute or chronic illness and to promote recovery of physical function and strength and promote mobility after an acute or chronic illness, or hip fracture. In other embodiments, the method provides benefits such as reducing prostate related side effects such as prostate cancer, enlarged prostate, hirsutism and acne. In some embodiments, the method selectively promotes beneficial effects of testosterone, while preventing side effects related to testosterone administration by at least 10%. In some methods, the testosterone or analogue thereof and ornithine decarboxylase (ODC) inhibitor are administered simultaneously. In some methods, the testosterone or analogue thereof and ornithine decarboxylase (ODC) inhibitor are administered sequentially. In some methods, the subject is a male or female. In some methods, the testosterone or analogue thereof; and an ornithine decarboxylase (ODC) inhibitor can be administered through oral, intravenous, intramuscular, transdermal, rectal, parenteral, intrathecal, vaginal routes or by direct absorption through mucous membrane. In some methods, said composition is administered in the form of gel, tablet, capsule, granulate, food product, troches, dispersions, suspensions solutions, or patches.

Additional methods include methods for treating a subject with low testosterone levels by selectively promoting beneficial effects of testosterone, while preventing side effects related to testosterone administration, comprising administration of testosterone or analogue thereof and an ornithine decarboxylase (ODC) inhibitor. In some methods, the subject has testosterone level of less than 350 pg/ml, or age-related low testosterone production, or the subject suffers from a low-testosterone associated disorder (e.g., type 2 diabetes, metabolic syndrome, dyslipidemia, obesity-related insulin resistance or hyperinsulinemia, male menopause, andropause, hypogonadotropichypogonadism, testicular failure, hyperprolactinemia, hypopituitarism, hypothalamic or pituitary disease, genetic diseases such as Klinefelter's syndrome, Kallman's syndrome, and Prader-Willi syndrome, infertility, myotonic dystrophy, acquired damage to the testes, such as alcoholism, physical injury, or viral diseases like mumps or testicular cancer). In some of such methods, the ornithine decarboxylase inhibitor is selected from the group consisting of 2-difluoromethylornithine, N-(4′-Pyridoxyl)-Ornithine(BOC)-OMe(POB), α-methyl ornithine, 1,4-diamino-2-butanone (DAB), and antizyme (AZ) or combination thereof. In some embodiments, the testosterone analogue is selected from the group consisting of testosterone ester, testosterone salts, testosterone prodrugs, fatty acid ester of testosterone, and testosterone metabolites. In some of such methods, the testosterone analogue is selected from the group comprising of testosterone ester, testosterone salts, testosterone prodrugs, fatty acid ester of testosterone, and testosterone metabolites. In some of these methods, the ester of testosterone is a C2-C13 alkyl ester or an undecanoate acid ester of testosterone. In some embodiments, the testosterone or analogue thereof and ODC inhibitor are each administered in an amount of about 0.0002 mg/kg to about 50 mg/kg of body weight per day. In some of such methods, the side effects that are reduced include prostate related side effects in males and virilization in females. In some embodiments, the beneficial effects comprise improvement in muscle mass and muscle performance, and physical function or measures of physical function in subjects with sarcopenia, frailty, or falls, or increased risk of falls. In other methods, the beneficial effects include improved mobility in subjects with mobility limitation associated with sarcopenia, muscle atrophy and weakness due to acute or chronic illness, bed rest, or fracture. In more embodiments, the beneficial effects include treating muscle wasting and physical dysfunction in subjects who have muscle wasting due to bed rest, immobilization, surgery, or placement of an orthopedic cast or because of any acute or chronic illness and to promote recovery of physical function and strength and promote mobility after an acute or chronic illness, or hip fracture. In other embodiments, the prostate related side effects that are reduced by said methods are prostate cancer, enlarged prostate, hirsutism and acne. In some embodiments, these methods selectively promote beneficial effects of testosterone, while preventing side effects related to testosterone administration by at least 10%. In some of such methods, the testosterone or analogue thereof and ornithine decarboxylase (ODC) inhibitor are administered simultaneously. In some methods, the testosterone or analogue thereof and ornithine decarboxylase (ODC) inhibitor are administered sequentially. In some methods, the subject is a male or female. In some methods, the testosterone or analogue thereof; and an ornithine decarboxylase (ODC) inhibitor can be administered through oral, intravenous, intramuscular, transdermal, rectal, parenteral, intrathecal, vaginal routes or by direct absorption through mucous membrane. In some methods, said composition is administered in the form of gel, tablet, capsule, granulate, food product, troches, dispersions, suspensions solutions, or patches.

Additional methods include methods of treating or inhibiting hypogonadism in males by selectively promoting beneficial effects of testosterone, while preventing prostate related side effects, comprising administration of testosterone or analogue thereof; and an ornithine decarboxylase (ODC) inhibitor.

More embodiments include a diagnostic kit comprising a combination of testosterone or analogue thereof; and an ornithine decarboxylase (ODC) inhibitor, which can include instructions for use thereof.

### BRIEF DESCRIPTION OF THE DRAWINGS

FIGS. 1A-1D shows prior-art images from Bhasin et al., (Nature CPEM, 2005) and Tracz et al., (JCEM, 206) showing that testosterone improves lean body mass (LBM), a measure of muscle mass, muscle strength and bone mineral density (BMD) and reduces fat mass. FIG. 1A shows mean difference in LBM (kg) change in men treated with testosterone and those treated with placebo in previous randomized trials of older men with low or low normal testosterone levels. FIG. 1B shows mean difference in grip strength (kg) change with testosterone in men treated with testosterone and those treated with placebo in previous randomized trials of older men with low or low normal testosterone levels. FIG. 1C shows mean difference in fat mass (kg) with testosterone in men treated with testosterone and those treated with placebo in previous randomized trials of older men with low or low normal testosterone levels. FIG. 1D shows mean difference in vertebral bone mineral density (BMD) with testosterone in men treated with testosterone and those treated with placebo in previous randomized trials of older men with low or low normal testosterone levels.

FIG. 2 is a table of results from a meta-analysis of adverse side effects in testosterone replacement clinical trials, showing adverse side-effects associated with prostate are a major concern.

FIG. 3 is a schematic diagram showing the mechanisms by which testosterone and other androgens exert their effects. The diagram makes the point that upon binding to its ligand, testosterone, androgen receptor associates with beta-catenin, translocates into the nucleus, and binds TCF-4, resulting in activation of a number of Wnt-target genes, including follistatin. Follistatin mediates the effects of testosterone on the muscle and bone. The diagram illustrates the identification of follistatin as an androgen regulated transcript.

FIG. 4A-4B show prior-art images of a superarray analysis showing that testosterone activates a number of Wnt target genes (Singh et al., Endocrinology, 2006), including follistatin. FIG. 4A shows the superarray analysis of 3T3-L1 cells in the presence and absence of testosterone and identifies upregulated genes in the testosterone treated cells (Right panel). FIG. 4B is a graph showing the upregulation of Fst (follastin) and CD44 and Lef1 gene expression when 3T3-L1 cells are incubated with 100 nM testosterone.

FIG. 5A-5D show prior art images demonstrating anti-follistatin antibody blocks the effects of testosterone on myogenic differentiation of mesenchymal multipotent cells, thus establishing an important role of follistatin in mediating the effects of testosterone on myogenic differentiation. (Singh et al., Endocrinology. 2009 March; 150(3): 1259-1268). FIG. 5A shows that testosterone (upper right panel) and follistatin (lower right panel) stimulate myogenic differentiation of mesenchymal multipotent cells (C3H 10T1/2 cells). C3H 10T1/2 cells were treated with testosterone (T) or Follistatin (rFST) or the combination of testosterone and ant-follistatin antibody (T+Fst ab) as compared to control (Con) and allowed to differentiate in myogenic medium for 3 days. Cells were fixed with 2% paraformaldehyde, and immunocytochemical analysis was performed using anti-MyoD antibody. The experiment was performed three times, and a representative photograph is shown. Anti-follistatin antibody blocked the effects of testosterone on myogenic differentiation. FIG. 5B shows a quantitative image analysis of MyoD+cells showing the number of MyoD-positive nuclei in mesenchymal multipotent cells after treatment with testosterone (T) or follistatin (rFST) or the combination of testosterone and anti-follistatin (T+Fst ab) as compared to control (Con). Three independent experiments were performed, and data are mean±sem. **, P≦0.001 for comparisons of groups, as shown in the figure. Statistical analysis was performed by using ANOVA; pairwise comparisons between groups were performed by Tukey's procedure. FIG. 5C shows cells were treated for 12 d, and immunocytochemical analysis was performed using anti-MHC II antibody. The experiment was performed three times, and a representative photograph is shown. Magnification, ×400. FIG. 5D shows a quantitative image analysis of MHC II+cells, showing IOD (Integrated OD which is area×average intensity). Three independent experiments were performed, and data are mean±sem. **, P≦0.01; #, P≦0.05. Statistical analysis was performed by ANOVA; pairwise comparisons between groups were performed by Tukey's procedure. Con, Control group treated with vehicle; T, 100 nm testosterone; T+Fst ab, 100 nm testosterone plus 0.5 μg/ml anti-Fst antibody; rFst, recombinant Fst protein (0.2 μg/ml).

FIGS. 6A-6C show the effect of systemic follistatin treatment (100 ug daily for 4-weeks) or vehicle (FST 0) on the muscle mass of treated mice. FIG. 6A shows images of the hindlimb of C57BL/6 mice treated with 0 or 100 μg recombinant follistatin daily for 28 days. FIG. 6B is a histogram of the fat mass, expressed as a percent of body weight, of mice after 13 weeks of systematic administration of 10, 30 and 100 μg follistatin, demonstrating that fat mass is decreased in follistatin treated animals. FIG. 6C shows a histogram of the lean mass of mice after 13 weeks of systemic administration of 0, 10, 30 and 100 μg of recombinant follistatin, demonstrating that lean body mass, measured by NMR is increased dose-dependently by graded doses of follistatin.

FIGS. 7A-7C show follistatin does not stimulate prostate growth, PSA or PCNA. FIG. 7A shows histologic sections of the prostate, showing atrophic prostate glands in castrated (CX) mice (upper right panel) compared to the vehicle-treated intact mice (upper left panel). Testosterone administration restored prostate mass and growth of prostate glands in castrated mice (lower right panel). In contrast, follistatin administration did not stimulate prostate growth or glands in castrated mice. FIG. 7B shows levels of PSA expression in the prostate epithelial cells treated with follistatin (BJS 1 ng/ml and 5 ng/ml) and 1 nM R1881 (an androgen), demonstrating that unlike and androgen which stimulates PSA expression, follistatin does not stimulate PSA expression in the prostate cells. FIG. 7C shows levels of PCNA expression in the prostate epithelial cells treated with follistatin (1 ng/ml and 5 ng/ml) and 10 nM DHT (Dihydrotestosterone, testosterone derivative). DHT upregulated PCNA expression, but follistatin did not.

FIG. 8A-8B show that follistatin and testosterone differentially regulate different sets of genes in the prostate of castrated mice, but share some commonly regulated genes in the skeletal muscle (levator ani). FIG. 8A is a schematic of microarray experiments to identify testosterone-selective signalling pathways in the prostate which are not activated by follistatin (Fst), and identify genes which are follistatin sensitive and those which are testosterone sensitive. Adult male mice were either sham-castrated or castrated under ketamine-xylazine anesthesia and treated with vehicle (sham), follistatin or testosterone. RNA was extracted from the prostate and levator ani muscle and subjected to microarrays. FIG. 8B shows a heat-map of differentially expressed genes. Shown in the box is the region of highly differentially expressed genes between the follistatin (Fst/CX) and testosterone (Tes/CX) treated mice. Testosterone was discovered to upregulate several biosynthetic pathways including the polyamine biosynthetic pathway, which were not upregulated in the follistatin treated mice. One gene upregulated by testosterone in the prostate in the polyamine biosynethic pathway is ornithine decarboxylase (ODC).

FIG. 9 shows that ODC inhibition retains testosterone-induced muscle anabolism while attenuating prostate growth. Shown is a histogram showing prostate weights and levator ani muscle weights in vehicle-treated intact C57BL/6 mice, and castrated mice treated with vehicle, testosterone, or testosterone plus an ODC inhibitor DFMO. Castration was associated with marked reduction in prostate and levator ani muscle mass, and testosterone administration restored prostate as well as levator ani muscle mass. In contrast, administration of testosterone in presence of ODC inhibitor DFMO, increased only levator ani muscle mass without restoring prostate mass. These data demonstrate that muscle and prostate growth are stimulated in castrated mice treated with testosterone alone, but only muscle growth, but not prostate growth, was enhanced in castrated mice treated a combination of testosterone and the ODC inhibitor DFMO. Accordingly, the ODC inhibitor DFMO suppresses testosterone-induced prostate growth in vivo, while maintaining muscle anabolic effects of testosterone.

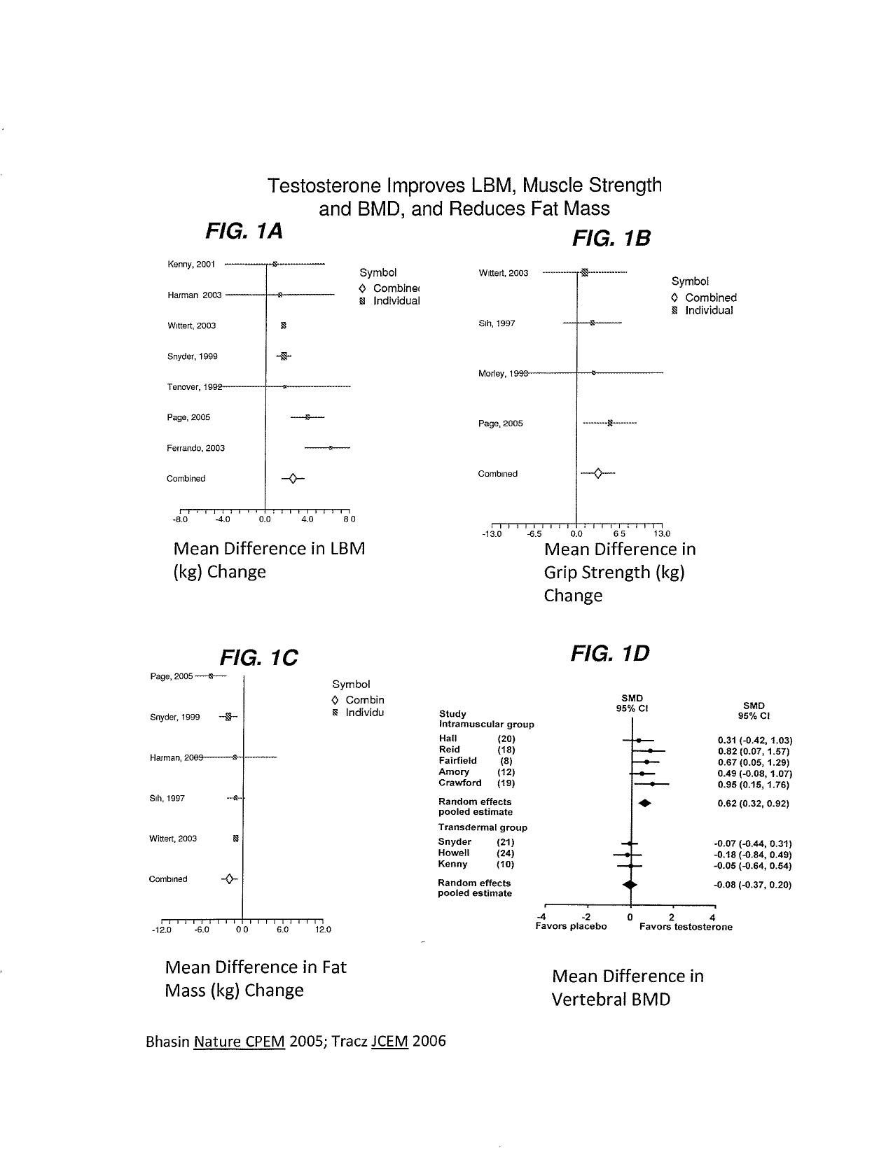

### DETAILED DESCRIPTION OF THE INVENTION

The present invention generally relates to the combination of testosterone or an analogue thereof and an ODC inhibitor, e.g., DFMO.

The inventors have surprisingly found that testosterone's effects on prostate growth require obligatory activation of the ornithine decarboxylase (ODC) and polyamine pathway, whereas testosterone's anabolic effects, e.g., on muscle growth do not require obligatory activation of ODC. The inventors have discovered that blocking this pathway by an ODC inhibitor, e.g., DFMO blocks testosterone' effects on the prostate but not on the skeletal muscle. Thus, one aspect of the present invention relates to the combined administration of testosterone plus an ODC inhibitor, e.g., DFMO as a unique approach to achieve selectivity of testosterone's beneficial effects on other organs and physiologic systems while sparing the prostate and preventing prostate-associated side effects.

The present invention is directed to a pharmaceutical composition comprising testosterone or analogue thereof; ornithine decarboxylase inhibitors. The composition of the invention selectively promotes beneficial effects of testosterone, while preventing side effects related to testosterone administration.

Another aspect of the present invention is directed to co-administrable combination of testosterone or analogue thereof in a first therapeutically effective amount, and an ornithine decarboxylase (ODC) inhibitor in a second therapeutically effective amount. The combination selectively promotes beneficial effects of testosterone, while preventing side effects related to testosterone administration.

Yet another aspect of the present invention is directed to a method for increasing testosterone levels in a subject comprising administration of; testosterone or analogue thereof in a first therapeutically effective amount; and an ornithine decarboxylase (ODC) in a second therapeutically effective amount. The method selectively promotes beneficial effects of testosterone, while preventing side effects related to testosterone administration.

The present invention relates to the combination of testosterone or an analogue or pharmaceutically acceptable salt thereof and a ODC inhibitor, e.g., DFMO which is useful for the treatment of subjects with low-testosterone associated disorders, or a variety of other disorders, such as, but not limited to, older men with low testosterone levels, older men with frailty or sarcopenia or other physical functional limitations, or in men with prostate cancer who are receiving androgen deprivation therapy, or who have undergone surgical orchiectomy, or who are hypogonadal for other reasons.

The present invention also relates to the combination of testosterone or an analogue or pharmaceutically acceptable salt thereof and an ODC inhibitor, e.g., DFMO as an androgen replacement therapy of older men with low testosterone levels, or in methods for the treatment of older persons with frailty, sarcopenia or any physical functional limitation such as mobility limitation.

In some embodiments, the present invention relates to the combination of testosterone or an analogue or pharmaceutically acceptable salt thereof and a ODC inhibitor, e.g., DFMO in methods for the treatment of men with prostate cancer who are receiving androgen deprivation therapy, or who have undergone surgical orchiectomy, or who are hypogonadal or have low testosterone for any other reason.

In some embodiments, the present invention relates to the combination of testosterone or an analogue or pharmaceutically acceptable salt thereof and an ODC inhibitor, e.g., DFMO as a method of testosterone replacement therapy of men at high risk of prostate cancer to minimize risk of prostate growth. In some embodiments, the present invention relates to the combination of testosterone or an analogue or pharmaceutically acceptable salt thereof and a ODC inhibitor, e.g., DFMO in a method of androgen replacement therapy of hypogonadal men.

Other aspects of the present invention relate to methods for increasing testosterone levels in a subject and methods for treating disorders and diseases associated with low testosterone levels in a subject, by administering a combination of testosterone and an ODC inhibitor, e.g., DFMO, as well as related kits for the above-described therapeutic uses.

Another aspect of the present invention relates to kits comprising testosterone or an analogue thereof and an ODC inhibitor, e.g., DFMO as disclosed herein, and instructions for administering the testosterone and an ODC inhibitor, e.g., DFMO to a subject in a combined matter to preserve the prostate function in the subject for the treatment of low testosterone levels in the subject, or to treat a testosterone associated disease or disorder for the treatment of low testosterone levels, or for the treatment to increase the testosterone levels, such as to increase testosterone level in a subject with male menopause, testicular cancer, testicle removal or AIDS.

Other aspects of the present invention relate to kits comprising a testosterone and an ODC inhibitor, e.g., DFMO as disclosed herein, and instructions for administering the testosterone in combination with an ODC inhibitor, e.g., DFMO to a subject to preserve prostate function for the treatment to subjects with low adrenal steroid or to increase the levels of adrenal steroid in the subject, or to treat a disease or disorder associated with low adrenal steroid, or for the treatment to increase adrenal steroid levels in a subject.

### DEFINITIONS

For convenience, certain terms employed herein, in the specification, examples and appended claims are collected here. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

The term “antagonist” or “inhibitor”, as used herein in reference to an ornithine decarboxylase (ODC) inhibitor, refers to a molecule which, when bound to ODC, decreases the amount or the duration of the effect of the biological or immunological activity of ODC. Antagonists may include proteins, nucleic acids, carbohydrates, antibodies or any other molecules that decrease the activity or the effect of ODC or the ODC pathway.

The term a “selective” ODC inhibitor as used herein refers to an agent that inhibits ODC activity with a Ki at least 10-fold less, preferably, at least 100-fold less, than the Ki for inhibition of a molecule which is not an ODC molecule.

The term “decreased ODC activity” means a substantial decrease by a statistically significant amount in the total ODC polypeptide activity of the ODC enzyme as a result of inhibition with a ODC inhibitor as disclosed herein as compared to in the absence of such inhibitor.

The term “biologically active”, as used herein, refers to a protein having structural, regulatory, or biochemical functions of a naturally occurring molecule, and displays the activity of the molecule in a cellular and/or in vivo assay.

The term “isomer” as used herein refers to a compound with the same molecular formula but different structural formulas. Isomers do not necessarily share similar properties, unless they also have the same functional groups. There are many different classes of isomers, like stereoisomers, enantiomers, geometrical isomers, etc. There are two main forms of isomerism: structural isomerism and stereoisomerism (spatial isomerism).

The phrase “combination therapy” (or “co-therapy”) embraces the administration of a testosterone (or an analogue, derivative or salt thereof) and an ODC inhibitor as part of a specific treatment regimen intended to provide a beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected). “Combination therapy” generally is not intended to encompass the administration of two or more of these therapeutic agents as part of separate monotherapy regimens that incidentally and arbitrarily result in the combinations of the present invention. “Combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. The sequence in which the therapeutic agents are administered is not narrowly critical. “Combination therapy” also can embrace the administration of the therapeutic agents as described above in further combination with other biologically active ingredients (such as, but not limited to, a second and different agent for treatment of low testosterone levels, and/or for treatment of prostate cancer etc.,) and non-drug therapies (such as, but not limited to, surgery or radiation treatment). Where the combination therapy further comprises radiation treatment, the radiation treatment may be conducted at any suitable time so long as a beneficial effect from the co-action of the combination of the therapeutic agents and radiation treatment is achieved. For example, in appropriate cases, the beneficial effect is still achieved when the radiation treatment is temporally removed from the administration of the therapeutic agents, perhaps by days or even weeks.

The phrases “low dose” or “low dose amount”, in characterizing a therapeutically effective amount of the testosterone therapy in the combination therapy, defines a quantity of such agent, or a range of quantity of such agent, that is capable of increasing testosterone levels in the subject while preventing or minimizing testosterone-induced prostate side-effects, such as but not limited to increases in prostate growth and/or prostate mass.

The term “treating”, as used herein, refers to altering the disease course of the subject being treated. Therapeutic effects of treatment include, without limitation, preventing occurrence or recurrence of disease, alleviation of symptom(s), diminishment of direct or indirect pathological consequences of the disease, decreasing the rate of disease progression, amelioration or palliation of the disease state, and remission or improved prognosis. In some embodiments, treating includes an increase in the level of testosterone, (e.g., total testosterone and/or free testosterone) in a subject, and/or an increase in the production of testosterone from Leydig cells in the subject. In some embodiments, treatment can be prophylactic treatment, for example, treatment of low testosterone levels for prevention of low-testosterone related diseases, for example, but not limited to type 2 diabetes, t metabolic syndrome, dyslipidemia, obesity, insulin resistance, hyperinsulinemia and the like.

The term “male sexual dysfunction” as used herein includes impotence, loss of libido, erectile dysfunction, and ejaculatory dysfunction.

The term “erectile dysfunction” as used herein refers to a disorder involving the failure of a male mammal to achieve erection, ejaculation, or both. Symptoms of erectile dysfunction include an inability to achieve or maintain an erection, ejaculatory failure, premature ejaculation, or inability to achieve an orgasm. An increase in erectile dysfunction is often associated with age and is generally caused by a physical disease or as a side-effect of drug treatment.

The term “pharmaceutically acceptable excipient”, as used herein, refers to carriers and vehicles that are compatible with the active ingredient (for example, a compound of the invention) of a pharmaceutical composition of the invention (and preferably capable of stabilizing it) and not deleterious to the subject to be treated. For example, solubilizing agents that form specific, more soluble complexes with the compounds of the invention can be utilized as pharmaceutical excipients for delivery of the compounds. Suitable carriers and vehicles are known to those of extraordinary skill in the art. The term “excipient” as used herein will encompass all such carriers, adjuvants, diluents, solvents, or other inactive additives. Suitable pharmaceutically acceptable excipients include, but are not limited to, water, salt solutions, alcohol, vegetable oils, polyethylene glycols, gelatin, lactose, amylose, magnesium stearate, talc, silicic acid, viscous paraffin, perfume oil, fatty acid monoglycerides and diglycerides, petroethral fatty acid esters, hydroxymethyl-cellulose, polyvinylpyrrolidone, etc. The pharmaceutical compositions of the invention can also be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like, which do not deleteriously react with the active compounds of the invention.

The terms “salts” and “pharmaceutically acceptable salts” refer to organic and inorganic salts of a compound, a stereoisomer of a compound, or a prodrug of a compound as disclosed herein. Thus, as used herein, the term “pharmaceutically acceptable salt,” is a salt formed from an acid and a basic group of a compound of the invention. Illustrative salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, and pamoate salts.

The term “pharmaceutically acceptable salt” also refers to a salt prepared from a compound as disclosed herein having an acidic functional group, such as a carboxylic acid functional group, and a pharmaceutically acceptable inorganic or organic base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl, N-ethylamine; diethylamine; triethylamine; mono-, bis-, or tris-(2-hydroxy-lower alkyl amines), such as mono-, bis-, or tris-(2-hydroxyethyl)amine, 2-hydroxy-tert-butylamine, or tris-(hydroxymethyl)methylamine, N,N,-di-lower alkyl-N-(hydroxy lower alkyl)-amines, such as N,N-dimethyl-N-(2-hydroxyethyl)amine, or tri-(2-hydroxyethyl)amine; N-methyl-D-glucamine; and amino acids such as arginine, lysine, and the like. Other pharmaceutically acceptable salts are described in the Handbook of Pharmaceutical Salts. Properties, Selection, and Use (P. Heinrich Stahl and C. Wermuth, Eds., Verlag Helvetica Chica Acta, Zurich, Switzerland (2002)).

The term “subject” as used herein refers to a vertebrate, preferably a mammal, more preferably a primate, still more preferably a human. Mammals include, without limitation, humans, primates, wild animals, feral animals, farm animals, sports animals, and pets. In some embodiment, a subject includes domestic and commercial farm animal, for example, but not limited to, cattle, pigs, horses and other commercial animals. In some embodiments, a subject is a male subject, however, subjects also include female subjects as well as subjects who are transgendered female to male subjects. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. A patient or subject includes any subset of the foregoing, e.g., all of the above. In certain embodiments of the aspects described herein, the subject is a mammal, e.g., a primate, e.g., a human. The terms, “patient” and “subject” are used interchangeably herein.

Mammals other than humans can be advantageously used as subjects that represent animal models of age related low testosterone levels, or conditions or disorders associated with low testosterone. In addition, the methods and compositions described herein can be used to treat domesticated animals and/or pets, for example, to increase testosterone production in male commercial animals, such as cattle, bison, and horses, such as male stud cattle and horses, and the like.

In some embodiments, a subject can be one who has been diagnosed with, or identified as has having a low testosterone level as determined by one of ordinary skill in the art and as disclosed herein, or a subject who is currently being treated for low testosterone levels, erectile dysfunction or male menopause. In some embodiments of the aspects described herein, the method further comprising diagnosing a subject for low testosterone levels by the methods as disclosed herein before beginning treatment with a method described herein. Methods of diagnosing low testosterone levels in a subject are well known in the art. In some embodiments, the method of treatment further comprises selecting a subject who has been identified or diagnosed with a low testosterone level before beginning treatment with a compound as disclosed herein according to the kits and methods as described herein.

The term “prodrug” refers to a compound that formulated as a precursor compound that, following administration, activates or releases the active component of the compound in vivo via a chemical or physiological process (e.g., upon being brought to physiological pH or through enzyme activity). A discussion of the synthesis and use of prodrugs is provided by Higuchi and Stella, Prodrugs as Novel Delivery Systems, vol. 14 of the ACS Symposium Series, and Bioreversible Carriers in Drug Design, ed. Edward B. Roche, American Pharmaceutical Association and Pergamon Press, 1987. Accordingly, the term “prodrug” refers to compounds that can be converted via some chemical or physiological process (e.g., enzymatic processes and metabolic hydrolysis) to an inactive form that can be activated in vivo by some co-compound or a specific environmental condition, e.g., pH etc. A prodrug may be inactive when administered to a subject, i.e. an ester, but is converted in vivo to an active compound, for example, by hydrolysis to the free carboxylic acid or free hydroxyl. The prodrug compound often offers advantages of solubility, tissue compatibility or delayed release in an organism. The term “prodrug” is also meant to include any covalently bonded carriers, which release the active compound in vivo when such prodrug is administered to a subject.

The term “therapeutically effective amount” as used herein refers to an amount sufficient to effect a beneficial or desired clinical result upon treatment. Specifically, the term “therapeutically effective amount” means an amount of a compound of this invention sufficient to measurably (i) increase the production of testosterone from Leydig cells from the subject, and/or (ii) increase the level of testosterone in a subject by a statistically significant level as compared to in the absence of a compound, where the level of testosterone refers either to free testosterone (FT) and/or total testosterone (TT), (iii) or using a cell-based assay as disclosed herein that increases the growth of fission yeast in 5FOA media expressing PDE8 after 48 hrs of incubation, and/or (iv) cause a measurable improvement in an animal model of a low testosterone levels, for example, increase testosterone level in Mice, that display age-dependent reduction in testosterone levels. Alternatively, a “therapeutically effective amount” is an amount of a compound as disclosed herein sufficient to confer a prophylactic effect on the treated subject against one or more of: dyslipidemia, obesity, metabolic syndrome, insulin resistance or hyperinsulinemia that are a consequence of a low testosterone level in the subject. Therapeutically effective amounts will vary, as recognized by those skilled in the art, depending on the specific disease treated, the route of administration, the excipient selected, and the possibility of combination therapy.

Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents. Furthermore, therapeutically effective amounts will vary, as recognized by those skilled in the art, depending on the specific disease treated, the route of administration, the excipient selected, and the possibility of combination therapy.

A physiological effects of a compound as disclosed herein on the subject can be measured to determine the therapeutically effective amount include, without limitation, levels of testosterone in a subject, gene induction (e.g., testosterone expression) and the like. Relevant assays to measure levels of testosterone include, with limitation, radioimmunoassay, immunofluorometric assays, immunochemiluscent assays, bioassays and mass spectrometry-based assays.

The term “pharmacologically effective amount” shall mean that amount of a drug or pharmaceutical agent that will elicit the biological or medical response of a tissue, system, animal or human that is being sought by a researcher or clinician. This amount can be a therapeutically effective amount.

The term “biologically active” refers to a compound that is capable of eliciting a biological response in a tissue, system, animal or human.

The term “pharmaceutically acceptable” is used herein to mean that the modified noun is appropriate for use in a pharmaceutical product. Pharmaceutically acceptable cations include metallic ions and organic ions. More preferred metallic ions include, but are not limited to, appropriate alkali metal salts, alkaline earth metal salts and other physiological acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include, without limitation, hydrochloric acid, hydroiodic acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid oxalacetic acid, fumaric acid, propionic acid, aspartic acid, glutamic acid, benzoic acid, and the like.

The phrase “administration” in defining the use of testosterone (or an analogue, derivative or salt thereof) and an ODC inhibitor, such as DFMO is intended to encompass administration of each agent individually, or together (e.g., combined in the same composition) in a manner and in a regimen that will provide beneficial effects of the drug combination therapy, and is intended to encompass co-administration of testosterone(or an analogue, derivative or salt thereof) in a substantially simultaneous manner with one or more, or 2 or more ODC inhibitors, such as DFMO in a substantially simultaneous manner, e.g., in a single capsule or dosage device having a fixed ratio of testosterone:ODC inhibitor, or alternatively in multiple, separate capsules or dosage devices for each agent (e.g., separate administration doses of testosterone and an ODC inhibitor), where the separate capsules or dosage devices can be taken together contemporaneously, or taken within a period of time sufficient to receive a beneficial effect or a therapeutically effective dose of testosterone (or an analogue, derivative or salt thereof) to increase the testosterone levels in the subject and a therapeutically effective dose of an ODC inhibitor, such as DFMO to inhibit the testosterone-induced side effects in the prostate.

The terms “decrease”, “reduced”, “reduction”, “decrease” or “inhibit” are all used herein generally to mean a decrease by a statistically significant amount. However, for avoidance of doubt, ““reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level (e.g., in the absence of a compound of the invention).

The terms “increased”, “increase” or “enhance” or “activate” are all used herein to generally mean an increase by a statistically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level (e.g., in the absence of a compound of the invention).

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

The term “substantially” as used herein means a proportion of at least about 60%, or preferably at least about 70% or at least about 80%, or at least about 90%, at least about 95%, at least about 97% or at least about 99% or more, or any integer between 70% and 100%.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth.

Other than in the operating examples, or where otherwise indicated, all numbers expressing quantities of ingredients or reaction conditions used herein should be understood as modified in all instances by the term “about.” The term “about” when used in connection with percentages can mean±1%.

In this application and the claims, the use of the singular includes the plural unless specifically stated otherwise. In addition, use of “or” means “and/or” unless stated otherwise. Moreover, the use of the term “including”, as well as other forms, such as “includes” and “included”, is not limiting. Also, terms such as “element” or “component” encompass both elements and components comprising one unit and elements and components that comprise more than one unit unless specifically stated otherwise.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology, and molecular biology can be found in The Merck Manual of Diagnosis and Therapy, 18th Edition, published by Merck Research Laboratories, 2006 (ISBN 0-911910-18-2); Robert S. Porter et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006. Definitions of common terms in molecular biology are found in Benjamin Lewin, Genes IX, published by Jones & Bartlett Publishing, 2007 (ISBN-13: 9780763740634); Kendrew et al. (eds.), The Encyclopedia of Molecular Biology, published by Blackwell Science Ltd., 1994 (ISBN 0-632-02182-9); and Robert A. Meyers (ed.), Maniatis et al., Molecular Cloning: A Laboratory Manual, Cold Spring Harbor Laboratory Press, Cold Spring Harbor, N.Y., USA (1982); Sambrook et al., Molecular Cloning: A Laboratory Manual (2 ed.), Cold Spring Harbor Laboratory Press, Cold Spring Harbor, N.Y., USA (1989); Davis et al., Basic Methods in Molecular Biology, Elsevier Science Publishing, Inc., New York, USA (1986); or Methods in Enzymology: Guide to Molecular Cloning Techniques Vol. 152, S. L. Berger and A. R. Kimmerl Eds., Academic Press Inc., San Diego, USA (1987); Current Protocols in Molecular Biology (CPMB) (Fred M. Ausubel, et al. ed., John Wiley and Sons, Inc.), Current Protocols in Protein Science (CPPS) (John E. Coligan, et. al., ed., John Wiley and Sons, Inc.) and Current Protocols in Immunology (CPI) (John E. Coligan, et. al., ed. John Wiley and Sons, Inc.), which are all incorporated by reference herein in their entireties.

It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

### Testosterone

Without wishing to be bound by theory, testosterone is commonly known to persons of ordinary skill in the art and is shown by compound with the formula:

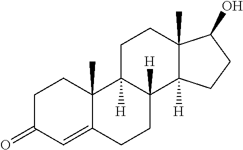

Testosterone is also known under the chemical name 17-.beta.-hydroxyandrost-4-en-3-one which can be obtained in various ways: it may be isolated and purified from nature or synthetically produced by any manner. Besides testosterone also an “analogue of testosterone” can be used in an embodiment of the present invention. The term “or analogue thereof” includes any useful metabolite or precursor of testosterone, for example the metabolite dihydrotestosterone. It is clear to the skilled person that if a metabolite or precursor of testosterone is used, the time point for administration of for example an ODC inhibitor needs to be reconsidered. If, for example, dihydrotestosterone (DHT) is used, the time of administration of an ODC inhibitor lies approximately half an hour earlier (as this is the approximate time it takes for excess testosterone to be converted to dihydrotestosterone).

In some embodiments, testosterone can be a derivative or analogue or a salt of testosterone. In some embodiments, testosterone analogue is a testosterone ester, prodrug or fatty acid ester of testosterone.

In some embodiments, a fatty acid ester of testosterone of long chain (i.e., 14 or more carbons) slow the rate of hydrolysis of the ester by esterases, releasing the testosterone into the system. In some embodiments, testosterone is an oral form of testosterone such as methyltestosterone (in which the methyl group is covalently bonded to the testosterone nucleus as the C17 position to inhibit hepatic metabolism). In some embodiments, testosterone for use in the methods and compositions as disclosed herein is disclosed in US Patent Application US2011/0251167 which is incorporated herein in its entirety by reference.

In some embodiments, testosterone for use in the methods and compositions as disclosed herein is testosterone undecanote. In some embodiments, testosterone for use in the methods and compositions as disclosed herein can be an undecanoate acid ester of testosterone (herein referred to as “TU”), other esters of hydrophobic compounds, including Testosterone (herein also referred to as “T”). In fact, it should be readily apparent to one of ordinary skill in the art from the teachings herein that the testosterone, alone or in combination with the ODC inhibitor as disclosed herein can be suitable for oral delivery of other testosterone esters, such as short-chain (C₂-C₆), medium-chain (C₇-C₁₃) and long-chain (C₁₄-C₂₄) fatty acid esters, preferably medium-chain fatty acid esters of testosterone.

In some embodiments, a testosterone analogue for use in the methods and compositions as disclosed herein is testosterone alkyl ester.

In some embodiments, any pharmaceutical composition described herein comprises about 10 mg to about 400 mg, or about 10 mg to about 1000 mg of testosterone alkyl ester. In some embodiments, any pharmaceutical composition as described herein comprises about 10 mg to about 300 mg of testosterone alkyl ester. In certain embodiments, any pharmaceutical composition as described herein comprises about 10 mg to about 240 mg of testosterone alkyl ester. In some embodiments, any pharmaceutical composition as described herein comprises about 10 mg to about 150 mg of testosterone alkyl ester.

In some embodiments, any pharmaceutical composition as described herein comprises about 120 mg of testosterone alkyl ester. In some embodiments, a testosterone analogue is C₂-C₁₃ alkyl ester.

The formulation of the testosterone or testosterone derivatives and analogues or salts thereof should provide a dose of testosterone adequate to maintain the male subject's serum total testosterone level within the normal male range (approximately 300 to 1000 ng/dL range), based on measures of serum total testosterone. The amount of the testosterone or testosterone derivatives and analogues or salts thereof present in the compositions as disclosed herein depends on the patient's starting serum total testosterone and the mode of administration. For oral administration, the compositions are preferably provided in the form of tablets containing 0.01, 0.05, 0.1, 0.5, 1.0, 2.5, 5.0, 10.0, 15.0, 25.0, 50.0 and 100 milligrams of active ingredient for the symptomatic adjustment of the dosage to the subject to be treated.

An effective amount of the drug is ordinarily supplied at a dosage level of from about 0.0002 mg/kg to about 50 mg/kg of body weight per day. The range is more particularly from about 0.001 to 7 mg/kg of body weight per day.

In particular, testosterone and testosterone derivatives and analogues or salts thereof delivered by intramuscular injections may be provided in injections of 50 to 750 mg every 1 to 4 weeks. In one embodiment, testosterone and testosterone derivatives and analogues or salts thereof are provided by intramuscular injections of 100 to 500 mg every one to 4 weeks. In one class of this embodiment, testosterone and testosterone derivatives and analogues or salts thereof are provided by intramuscular injections of 50 to 250 mg every 2 to 4 weeks.

Testosterone and testosterone derivatives and analogues or salts thereof may be provided in gel or cream forms in doses of 20 to 200 mg per day.

In one embodiment, testosterone and testosterone derivatives and analogues or salts thereof are provided in a gel at doses of 50 to 100 mg/day, particularly 50 mg/day, 75 mg/day and 100 mg/day.

Transdermal patches can be used to deliver testosterone and testosterone derivatives and analogues or salts thereof of 1 to 10 mg per day, particularly, 4 to 6 mg/day.

Testosterone, testosterone derivatives and analogues or salts thereof may also be provided by means of a buccal gel at a dose of 10 mg/day to 100 mg/day. In one embodiment, the dose of testosterone or testosterone derivatives and analogues or salts thereof is a buccal gel is 40 to 80 mg/day. In one class of this embodiment, the dose of testosterone or testosterone derivatives and analogues or salts thereof in a buccal gel is 60 mg/day.

In some embodiments, testosterone administered in combination with an ODC inhibitor as disclosed herein can be administered to a subject, e.g., a subject with a low-testosterone associated disorder, once- or twice-daily, e.g., via oral administration, provides an average serum testosterone concentration at steady state falling in the range of about 300 to about 1100 ng/dL. In some embodiments, the pharmaceutical composition provides a Cᵣₙₐₓ that, when administered with a meal, does not exceed 2500 ng/dL, preferably does not exceed 1800 ng/dL, and most preferably does not exceed 1500 ng/dL.

According to the invention the level of free testosterone should be a peak plasma level of free testosterone of about at least 0.010 nmol/L, which will typically occur between 1 and 20 minutes after administration of the testosterone. About three and a half to five and a half hours after this plasma testosterone peak, there is a testosterone effect peak, i.e. there is a time lag in the effect of testosterone on genital arousal in sexually functional women.

Provided in certain embodiments herein is testosterone composition comprising an ODC inhibitor that provides to a human in need of androgen therapy by delivering to the human a composition comprising a therapeutically effective amount of testosterone, testosterone analogue or derivative or salt thereof in combination with an ODC inhibitor. In some embodiments, a testosterone analogue is testosterone undecanoate (TU). In some embodiments, a composition comprising testosterone undecanoate provides in a human (e.g., a male human) a mean Cₘₐₓ of testosterone that is less than about 15 ng/mL; or less than about 19 ng/mL after a single administration of the composition. In certain embodiments, a composition comprising testosterone undecanoate provides to a human (e.g., a male human) a mean plasma Cₘₐₓ of dihydrotestosterone that is about 3.6 ng/mL or less; or about 4.5 ng/mL or less after a single administration of the composition. In some embodiments, a composition comprising testosterone undecanoate provides to a human (e.g., a male human) a testosterone mean plasma Cₘₐₓ at steady state of about 1300 ng/dL or less. In certain embodiments, a composition comprising testosterone undecanoate provides to a human (e.g., a male human) a testosterone mean plasma Cₘₐₓ at steady state of about 200 ng/dL or more. In some embodiments, a composition comprising testosterone undecanoate provides to a human (e.g., a male human) a mean Cₘₐₓ of testosterone at steady state to dose ratio of about 15 or less. In specific embodiments, the ratio is 15 or less, or 13 or less. In some embodiments provided herein is a pharmaceutical composition that provides with administration to an individual a ratio of a testosterone C₂-C₁₃ alkyl ester dose, in mg, to a mean steady state testosterone Cₘₐₓ, in mg/mL, the ratio of testosterone equivalent dose from the testosterone alkyl ester to a mean steady state testosterone Cₘₐₓ, the ratio being about 500.times.10.sup.6 mL or less (e.g., with b.i.d. or q.d. administration to an otherwise testosterone deficient individual). In certain embodiments, a composition comprising testosterone undecanoate provides to a human (e.g., a male human) a difference between a mean plasma Cₘₐₓ of testosterone at steady state and mean plasma Cₘᵢₙ of testosterone at steady state of about 11 ng/mL or less, or about 16 ng/mL or less. In some embodiments, a composition comprising testosterone undecanoate provides to a human (e.g., a male human) a difference between a mean plasma Cₘₐₓ at steady state and mean plasma Cₘᵢₙ at steady state of testosterone alkyl ester of about 200 ng/mL or less; or about 275 ng/mL or less. In certain instances, when a mean plasma concentration is utilized, the value is obtained from a statistically significant population of individuals.

In some embodiments, the testosterone C₂-C₁₃ alkyl ester is testosterone undecanoate. In certain embodiments, the pharmaceutical composition comprises about 10 mg to about 1000 mg of testosterone C₂-C₁₃ alkyl ester.

In some embodiments, a single dose of any pharmaceutical composition provided herein provides a mean plasma Cₘₐₓ of testosterone that is about 15 ng/mL or less; or about 19 ng/mL or less upon oral administration (e.g., to a testosterone deficient individual). In certain embodiments, a single dose of any pharmaceutical composition provided herein provides a mean plasma Cₘₐₓ of dihydrotestosterone that is about 4.5 ng/mL or less; or about 3.6 ng/mL or less upon oral administration (e.g., to a testosterone deficient individual). In some embodiments, any pharmaceutical composition provided herein provides a testosterone mean plasma C. at steady state of about 1300 ng/dL or less with oral administration (e.g., with b.i.d. or q.d. administration to an otherwise testosterone deficient individual). In certain embodiments, any pharmaceutical composition provided herein provides a testosterone mean plasma Cₘᵢₙ at steady state of about 200 ng/dL or more with oral administration (e.g., with b.i.d. or q.d. administration to an otherwise testosterone deficient individual). In some embodiments, any pharmaceutical composition provided herein provides with administration to an individual (e.g., oral administration) a ratio of testosterone equivalent dose from the testosterone alkyl ester to a mean a mean steady state testosterone Cₘₐₓ, the ratio being about 500×10⁶ mL, or less (e.g., with b.i.d. or q.d. administration to an otherwise testosterone deficient individual).

In some embodiments, the difference between the mean plasma Cₘₐₓ of testosterone at steady state and mean plasma Cₘᵢₙ of testosterone at steady state is about 11 ng/mL or less, or about 16 ng/mL or less (e.g., with b.i.d. or q.d. administration to an otherwise testosterone deficient individual). In some embodiments, the difference between the mean plasma Cₘₐₓ at steady state and mean plasma Cₘᵢₙ at steady state of testosterone C₂-C₁₃ alkyl ester is about 200 ng/mL or less; or about 275 ng/mL or less (e.g., with b.i.d. or q.d. administration to an otherwise testosterone deficient individual). In some embodiments, a single dose of any pharmaceutical composition provided herein provides a mean plasma concentration of testosterone after 1 hour that is about 150 ng/dL or less upon oral administration. In certain embodiments, a single dose of any pharmaceutical composition provided herein provides a mean plasma concentration of testosterone after 2 hours that is about 500 ng/dL or less upon oral administration.

In some embodiments, testosterone or derivatives or analogues or salts thereof are is a delayed release composition, e.g., an oral formulation, wherein a single dose of the delayed release oral dosage form provides a mean plasma Cₘₐₓ of testosterone that is at least 5% lower; or at least 10% lower than the mean plasma Cₘₐₓ of testosterone that is provided by a single dose of an immediate release oral dosage form having an identical amount of the testosterone C₂-C₁₃ alkyl ester. In some embodiments, the testosterone C₂-C₁₃ alkyl ester is testosterone undecanoate. In certain embodiments, the pharmaceutical composition comprises about 10 mg to about 1000 mg of testosterone C₂-C₁₃ alkyl ester.

In some embodiments, a single dose of any delayed release oral dosage form provided herein provides a mean plasma Cₘₐₓ of the that is at least 5%, at least 10% or at least 15% lower than the mean plasma Cₘₐₓ of testosterone C₂-C₁₃ alkyl ester that is provided by a single dose of an immediate release oral dosage form having an identical amount of the testosterone C₂-C₁₃ alkyl ester. In some embodiments, a single administration to a human of a dose of the delayed release oral dosage form provides a ratio of testosterone equivalent dose from the C₂-C₁₃ alkyl ester present in the dose of the delayed release oral dosage form to mean plasma testosterone Cₘₐₓ provided by the single administration of the dose of the delayed oral release dosage form, the ratio being about 500×10⁶ mL or less. In certain embodiments, a single dose of any delayed release oral dosage form provided herein provides a mean plasma Cₘₐₓ of that is at least 5% lower than the mean plasma Cₘₐₓ of dihydrotestosterone provided by a single dose of an immediate release oral dosage form having an identical amount of the testosterone C₂-C₁₃ alkyl ester. In some embodiments, a single administration to a human a dose of the delayed release oral dosage form provides a ratio of testosterone equivalent dose from the C₂-C₁₃ alkyl ester to mean plasma dihydroxytestosterone Cₘₐₓ provided by the single administration of the dose of the delayed oral release dosage form, the ratio being about 350×10⁶ mL or less. In some embodiments, any delayed release oral dosage form provided herein provides a mean plasma Cₘₐₓ at steady state of testosterone C₂-C₁₃ alkyl ester that is at least 5% lower, or at least 10% lower than mean plasma Cₘₐₓ of testosterone C₂-C₁₃ alkyl ester at steady state provided by an immediate release oral dosage form having an identical amount of the testosterone C₂-C₁₃ alkyl ester (e.g., when orally administered to a testosterone deficient individual b.i.d. or q.d.).

In certain embodiments, any delayed release oral dosage form provided herein comprises at least one pharmaceutically acceptable carrier that comprises at least one hydrophilic carrier. In specific embodiments, the hydrophilic carrier is a hydrophilic triglyceride. In more specific embodiments, the hydrophilic triglyceride is a polyoxylated castor oil, or a polyoxylated hydrogenated castor oil. In some embodiments, any delayed release oral dosage form provided herein consists essentially of a lipophilic carrier or combination of lipophilic carriers. In some embodiments, a lipophilic carrier selected from the group consisting of a monoglyceride, a diglyceride, a Vitamin E compound, a triglyceride, a fatty acid, polyoxylated fatty acid, polyoxylated triglyceride, polyoxylated vegetable oil, and a combination thereof. In certain embodiments, any delayed release oral dosage form provided herein comprises a lipophilic carrier and less than 10% w/w or less than 5% w/w of a hydrophilic carrier.

Testosterone can be given in a formulation wherein there is a short-lasting high peak of testosterone in the blood circulation of the subject to whom it is administered. The invention therefore provides a use, wherein the testosterone or an analogue thereof is provided in the form of a sublingual formulation, such as a sublingual formulation comprising cyclodextrins as carrier. Another example of a suitable route of administration is buco-mucosally or intranasally, which can also be performed with the use of a cyclodextrin formulation or other usual excipients, diluents and the like. A typical example of a formulation is given in hydroxypropyl-beta cyclodextrin, but other beta cyclodextrins and other usual excipients, diluents and the like are within the skill of the art for preparing a formulation comprising testosterone or an analogue thereof, which releases essentially all of the testosterone within one short burst. Said burst will typically be within a short time interval (for example within 60-120 seconds, more preferably within 60 seconds) upon administration, leading to blood peak levels of testosterone about 1-20 minutes later. A typical example of a prepared testosterone sample (for 0.5 mg of testosterone) consists of 0.5 mg testosterone, 5 mg hydroxypropyl-betacyclodextrines (carrier), 5 mg ethanol, and 5 ml water, but each of the amounts of these substances might be higher or lower.

Testosterone in the circulation is typically bound by SHBG (steroid hormone binding globulin) and by albumin. It is important that the peak plasma level of testosterone as defined in the present invention is present and calculated as free testosterone, so a fraction not bound by albumin and SHBG. Thus the dose of testosterone given should be high enough to saturate the albumin and SHBG (i.e. the concentration of testosterone must be high enough to overcome complete binding of testosterone by SHBG or albumin), or another way of avoiding binding to albumin or SHBG must be designed, such as the use of a competitor for the testosterone binding site on SHBG.

In contrast to other sexual dysfunction treatments based on testosterone, the use (and method) described herein aim at a temporary increase in the testosterone level in the treated subject. Most other methods aim at restoring/replacing/replenishing of the testosterone level to normal (i.e. physiological) levels (as found in a normal subject). In a preferred embodiment, testosterone is applied such that a short-lasting high peak of testosterone in the blood circulation of the subject to whom it is administered, is obtained. The term “short-lasting” refers to an application of testosterone such that the blood serum testosterone levels are back to base-line level within 2 hours after administration.

Ornithine Decarboxylase Inhibitors

Without wishing to be bound by theory, the enzyme ornithine decarboxylase (OCD) catalyzes the decarboxylation of ornithine to putrescine, which is the first step in the biosynthesis of the polyamines known as spermidine and spermine. Spermidine is formed by the transfer of an activated aminopropyl moiety from S-adenosyl S-methyl homocysteamine to putrescine, while spermine is formed by the transfer of a second aminopropyl group to spermidine. S-Adenosyl S-methyl homocysteamine is formed by the decarboxylation of S-adenosylmethionine (SAM), a reaction catalyzed by the enzyme S-adenosylmethionine decarboxylase (SAM-DC).

The polyamines, which are found in animal tissues and microorganisms, are known to play an important role in cell growth and proliferation. The onset of cell growth and proliferation is associated with both a marked increase in ODC activity and an increase in the levels of putrescine and the polyamines. Although the exact mechanism of the role of the polyamines in cell growth and proliferation is not known, it appears that the polyamines may facilitate macromolecular processes such as DNA, RNA, or protein synthesis. Polyamine levels are known to be high in embryonic tissue; in the testes, ventral prostrate, and thymus; in tumor tissue; in psoriatic skin lesions; and in other cells undergoing rapid growth or proliferation.

Ornithine decarboxylase (ODC) is elevated in most tumors and premalignant lesions. Induction of cell growth and proliferation is associated with dramatic increases in ornithine decarboxylase activity and subsequent polyamine synthesis. Further, blocking the formation of polyamines slows or arrests growth in transformed cells. Consequently, polyamines are thought to play a role in tumor growth.

In one embodiment, an ornithine decarboxylase inhibitor for use in the methods and compositions as disclosed herein is eflornithine (also referred to herein as α-difluoromethylornithine or DFMO). DFMO is also referred to as (RS)-2,5-diamino-2-(difluoromethyl)pentanoic acid and is commonly known to persons of ordinary skill in the art and is shown by compound with the formula:

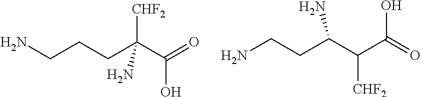 [SMILES: NCCC[C@](N)(C(=O)O)C(F)F ; NCC[C@H](N)C(C(=O)O)C(F)F]

In some embodiments, an ornithine decarboxylase inhibitor is an eflornithine hydrochloride, e.g., in the form of a cream, and is commercially available under the brand name VANIQA™ by Almirall in Europe, CSL in Australia, Triton in Canada, Mediso in Israel and SkinMedica in the USA. In some embodiments, an ODC inhibitor can be eflornithine in the formulation as injection, for example, available under the brand name ORNIDYL™ in the USA. Other forms of DFMO are encompassed in the methods and compositions as disclosed herein, and include an eflornithine hydrochloride, such as ORNIDYL™.

In some embodiments of the invention the ornithine decarboxylase inhibitor is 1,4-diamino-2-butanone (DAB).

In some embodiments, ornithine decarboxylase inhibitors can be used, for example, as disclosed in U.S. Pat. No. 4,720,489 which is incorporated herein in its entirety by reference. Among the ODC inhibitors which may be employed in the present invention are described in U.S. Pat. Nos. 4,201,788; 4,413,141; and 4,421,768, which are incorporated herein in their entirety by reference. In some embodiments, ODC inhibitors for use include 2(difluoromethyl)-2,5-diaminopentanoic acid; alpha-ethynyl ornithine; 6-heptyne-2,5-diamine; and 2-methyl-6-heptyne diamine.

Difluoromethylornithine (DFMO) is a potent inhibitor of ornithine decarboxylase that has been shown to inhibit carcinogen-induced cancer development in a variety of rodent models (Meyskens et al. Development of Difluoromethylornithine (DFMO) as a chemoprevention agent. Clin. Cancer Res. May 1999, 5(%): 945-951, hereby incorporated by reference, herein). DFMO is also known as 2-difluoromethyl-2,5-diaminopentanoic acid, or 2-difluoromethyl-2,5-diami-novaleric acid, or a-(difluoromethyl) ornithine; DFMO is marketed under the tradename ELFORNITHINE™. Therefore, the use of DFMO in combination with testosterone, or testosterone analogues, derivatives or salts thereof is contemplated to treat low testosterone disorders and diseases, without having testosterone-induced prostate side effects which typically occur with administration of testosterone alone.

Other compounds that can serve as the ornithine decarboxylase inhibitor for use in the methods and compositions as disclosed herein are caffeic acid, chlorogenic acid, and ferulic acid, (See, e.g., Hagerman, A. E., Tannin-Protein Interactions, Ch. 19 in Phenolic Compounds in Food and their Effects of Health, Ho, C. T. et al., Eds, (1985)); antizyme 1 and antizyme 2, (See, e.g., Zhu, C. et al., J. Biol. Chem., 274(37): 26425-26430 (1999)); DL-alpha-monofluoromethyldehydroornithine methyl ester, (See, e.g., Hollingdale, M. R., et al., Exp. Parasitol., 60(1): 111-117 (1985)); and 3-amino-oxy-1-propanamine, (See, e.g., Mett, H., et al., Cancer Chemother. Pharmacol., 32:39-45 (1993)).

### Compositions Comprising Testosterone or Analogues or Derivatives Thereof and ODC Inhibitors

An aspect of the present invention is directed to a pharmaceutical composition comprising testosterone or analogue thereof; ornithine decarboxylase inhibitors, wherein said composition selectively promotes beneficial effects of testosterone, while preventing side effects related to testosterone administration.

In some embodiments, a composition as disclosed herein for use in the methods as disclosed herein comprises testosterone or an analogue thereof, for use in combination with an ornithine decarboxylase (ODC) inhibitor, e.g., DFMO as disclosed above.

In some embodiments, a composition comprising testosterone or an analogue thereof, for use in combination with an ornithine decarboxylase (ODC) inhibitor, e.g., DFMO comprises at least about 80%, or at least about 85%, or at least about 90%, or at least about 92%, or at least about 95%, or at least about 97%, or at least about 98%, or at least about 99%, or at least about 99.5%, or at least about 99.8% or more than 99.8% of one or more of testosterone or an analogue or derivative or salt thereof or and ODC inhibitor.

As used herein, the term “composition” is intended to encompass a product comprising the specified ingredients in the specified amounts, as well as any product which results, directly or indirectly, from combinations of the specified ingredients in the specified amounts.

Where the processes for the preparation of the compounds according to the invention give rise to mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

In an embodiment of the present invention the testosterone analogues are selected from the group consisting of testosterone ester, testosterone salts, testosterone prodrugs, fatty acid ester of testosterone, testosterone metabolites.

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Furthermore, some of the crystalline forms for the compounds may exist as polymorphs and as such are intended to be included in the present invention. In addition, some of the compounds may form solvates with water (i.e., hydrates) or common organic solvents, and such solvates are also intended to be encompassed within the scope of this invention.

All stereoisomers of the present compounds, including enantiomeric and diastereomeric forms, are contemplated within the scope of this invention. Individual stereoisomers of the compounds of the invention may, for example, be substantially free of other isomers, or may be admixed with all other, or other selected, stereoisomers. The chiral centers of the present invention can have the S or R configuration as defined by the IUPAC 1974 Recommendations.

One skilled in the art will recognize that wherein a reaction step of the present invention may be carried out in a variety of solvents or solvent systems, said reaction step may also be carried out in a mixture of the suitable solvents or solvent systems.

It will also be appreciated by those skilled in the art that the compounds of the present invention may also be utilized in the form of their pharmaceutically acceptable salts or solvates thereof. The pharmaceutically acceptable salts of the compounds of the present invention are in particular salts which are non-toxic, or which can be used physiologically.

Thus, when a compound of the present invention comprising testosterone or an analogue or derivative or salt thereof, for use in combination with an ornithine decarboxylase (ODC) inhibitor, e.g., DFMO, contain one or more basic groups, i.e. groups which can be protonated, they can form an addition salt with a non-toxic inorganic or organic acid. Examples of suitable inorganic acids include: boric acid, perchloric acid, hydrochloric acid, hydrobromic acid, hydrofluoric acid, sulfuric acid, sulfamic acid, phosphoric acid, nitric acid and other inorganic acids known to the person skilled in the art. Examples of suitable organic acids include: acetic acid, propionic acid, succinic acid, glycolic acid, stearic acid, lactic acid, malic acid, tartaric acid, citric acid, ascorbic acid, pamoic acid, maleic acid, hydroxymaleic acid, fumaric acid, phenylacetic acid, glutamic acid, benzoic acid, salicylic acid, sulfanilic acid, 2-acetoxybenzoic acid, toluenesulfonic acid, methanesulfonic acid, benzenesulfonic acid, ethane disulfonic acid, oxalic acid, isethionic acid, ketoglutaric acid, glycerophosphoric acid, aspartic acid, picric acid, lauric acid, palmitic acid, cholic acid, pantothenic acid, alginic acid, naphthoic acid, mandelic acid, tannic acid, camphoric acid and other organic acids known to the person skilled in the art. Preferred salts include methane sulphonic acid, hydrochloric acid and p-toluenesulphonic acid salts.

Thus, a compound of the present invention comprising testosterone or an analogue or derivative or salt thereof, can contain an acidic group they can form an addition salt with a suitable base. For example, such salts of the compounds of the present invention may include their alkali metal salts, such as Li, Na and K salts, or alkaline earth metal salts, like Ca and Mg salts, or aluminium salts, or salts with ammonia or salts of organic bases, such as lysine, arginine, guanidine, diethanolamine, choline and tromethamine.

As disclosed herein, a testosterone or an analogue or derivative or salt thereof, for use in combination with an ornithine decarboxylase (ODC) inhibitor can be formulated as a salt, prodrug and solvate. The term “salt(s)”, as employed herein, denotes acidic and/or basic salts formed with inorganic and/or organic acids and bases and Zwitterions (internal or inner salts) are also included. Also included herein are quaternary ammonium salts such as alkylammonium salts. Pharmaceutically acceptable (i.e., non-toxic, physiologically acceptable) salts are preferred.

Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, 2-hydroxyethanesulfonates, lactates, maleates, methanesulfonates, 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates, undecanoates, and the like.

Exemplary basic salts include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines, N-methyl-D-glucamines, N-methyl-D-glucamides, t-butyl amines, and salts with amino acids such as arginine, lysine and the like.

The pharmaceutically acceptable salts of a testosterone or an analogue or derivative or salt thereof, or an ornithine decarboxylase (ODC) inhibitor can be synthesized to contain a basic or an acidic moiety, by conventional chemical methods. Generally, the salts are prepared by contacting the free base or acid with stoichiometric amounts or with an excess of the desired salt-forming inorganic or organic acid or base in a suitable solvent or dispersant or from another salt by cation or anion exchange. Suitable solvents are, for example, ethyl acetate, ether, alcohols, acetone, THF, dioxane, or mixtures of these solvents.

For use in medicine, the salts of the compounds of this invention refer to non-toxic “pharmaceutically acceptable salts.” Other salts may, however, be useful in the preparation of compounds according to this invention or of their pharmaceutically acceptable salts. Suitable pharmaceutically acceptable salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts.

Thus, representative pharmaceutically acceptable salts include the following: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate.

Representative acids and bases which may be used in the preparation of pharmaceutically acceptable salts include the following: acids including acetic acid, 2,2-dichloroactic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, (+)-(1S)-camphor-10-sulfonic acid, capric acid, caproic acid, caprylic acid, cinnamic acid, citric acid, cyclamic acid, dodecylsulfuric acid, ethane-1,2-disulfonic acid, ethanesulfonic acid, 2-hydrocy-ethanesulfonic acid, formic acid, fumaric acid, galactaric acid, gentisic acid, glucoheptonic acid, D-gluconic acid, D-glucoronic acid, L-glutamic acid, α-oxo-glutaric acid, glycolic acid, hipuric acid, hydrobromic acid, hydrochloric acid, (+)-L-lactic acid, (±)-DL-lactic acid, lactobionic acid, maleic acid, (−)-L-malic acid, malonic acid, (+−)-DL-mandelic acid, methanesulfonic acid, naphthalene-2-sulfonic acid, naphthalene-1,5-disulfonic acid, 1-hydroxy-2-naphthoic acid, nicotinc acid, nitric acid, oleic acid, orotic acid, oxalic acid, palmitric acid, pamoic acid, phosphoric acid, L-pyroglutamic acid, salicylic acid, 4-amino-salicylic acid, sebaic acid, stearic acid, succinic acid, sulfuric acid, tannic acid, (+)-L-tartaric acid, thiocyanic acid, p-toluenesulfonic acid and undecylenic acid; and bases including ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

Prodrugs and solvates of a testosterone analogue or derivative or salt thereof, or an ornithine decarboxylase (ODC) inhibitor as disclosed herein are also contemplated herein. The term “prodrug”, as employed herein, denotes a compound which, upon administration to a subject, undergoes chemical conversion by metabolic or chemical processes to yield a compound described herein or a salt and/or solvate thereof.

Thus, the present invention includes within its scope prodrugs of the compounds of this invention. In general, such prodrugs will be functional derivatives of the compounds which are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs”, ed. H. Bundgaard, Elsevier, 1985.

Accordingly, in some embodiments, compounds of a testosterone analogue or derivative or salt thereof, or an ornithine decarboxylase (ODC) inhibitor as disclosed herein are prodrugs, and can become activated in vivo upon predefined chemical modifications. Prodrugs of an active compound of a testosterone analogue or derivative or salt thereof, or an ornithine decarboxylase (ODC) inhibitor can be prepared by modifying functional groups present in the active compound in such a way that the modifications are cleaved, either in routine manipulation or in vivo, to the parent active compound. Prodrugs of a testosterone analogue or derivative or salt thereof, or an ornithine decarboxylase (ODC) inhibitor include compounds wherein a hydroxy, amino or mercapto group is bonded to any group that, when the prodrug of the active compound is administered to a subject, cleaves to form a free hydroxy, free amino or free mercapto group, respectively. Examples of prodrugs include, but are not limited to, acetate, formate and benzoate derivatives of an alcohol or acetamide, formamide and benzamide derivatives of an amine functional group in the active compound and the like. See Harper, “Drug Latentiation” in Jucker, ed. Progress in Drug Research 4:221-294 (1962); Morozowich et al, “Application of Physical Organic Principles to Prodrug Design” in E. B. Roche ed. Design of Biopharmaceutical Properties through Prodrugs and Analogs, APHA Acad. Pharm. Sci. 40 (1977); Bioreversible Carriers in Drug in Drug Design, Theory and Application, E. B. Roche, ed., APHA Acad. Pharm. Sci. (1987); Design of Prodrugs, H. Bundgaard, Elsevier (1985); Wang et al. “Prodrug approaches to the improved delivery of peptide drug” in Curr. Pharm. Design. 5(4):265-287 (1999); Pauletti et al. (1997) Improvement in peptide bioavailability: Peptidomimetics and Prodrug Strategies, Adv. Drug. Delivery Rev. 27:235-256; Mizen et al. (1998) “The Use of Esters as Prodrugs for Oral Delivery of (3-Lactam antibiotics,” Pharm. Biotech. 11,:345-365; Gaignault et al. (1996) “Designing Prodrugs and Bioprecursors I. Carrier Prodrugs,” Pract. Med. Chem. 671-696; Asgharnejad, “Improving Oral Drug Transport”, in Transport Processes in Pharmaceutical Systems, G. L. Amidon, P. I. Lee and E. M. Topp, Eds., Marcell Dekker, p. 185-218 (2000); Balant et al., “Prodrugs for the improvement of drug absorption via different routes of administration”, Eur. J. Drug Metab. Pharmacokinet., 15(2): 143-53 (1990); Balimane and S inko, “Involvement of multiple transporters in the oral absorption of nucleoside analogues”, Adv. Drug Delivery Rev., 39(1-3): 183-209 (1999); Browne, “Fosphenyloin (Cerebyx)”, Clin. Neuropharmacol. 20(1): 1-12 (1997); Bundgaard, “Bioreversible derivatization of drugs—principle and applicability to improve the therapeutic effects of drugs”, Arch. Pharm. Chemi 86(1): 1-39 (1979); Bundgaard H. “Improved drug delivery by the prodrug approach”, Controlled Drug Delivery 17: 179-96 (1987); Bundgaard H. “Prodrugs as a means to improve the delivery of peptide drugs”, Arfv. Drug Delivery Rev. 8(1): 1-38 (1992); Fleisher et al. “Improved oral drug delivery: solubility limitations overcome by the use of prodrugs”, Arfv. Drug Delivery Rev. 19(2): 115-130 (1996); Fleisher et al. “Design of prodrugs for improved gastrointestinal absorption by intestinal enzyme targeting”, Methods Enzymol. 112 (Drug Enzyme Targeting, Pt. A): 360-81, (1985); Farquhar D, et al., “Biologically Reversible Phosphate-Protective Groups”, Pharm. Sci., 72(3): 324-325 (1983); Freeman S, et al., “Bioreversible Protection for the Phospho Group: Chemical Stability and Bioactivation of Di(4-acetoxy-benzyl) Methylphosphonate with Carboxyesterase,” Chem. Soc., Chem. Commun., 875-877 (1991); Friis and Bundgaard, “Prodrugs of phosphates and phosphonates: Novel lipophilic alphaacyloxyalkyl ester derivatives of phosphate- or phosphonate containing drugs masking the negative charges of these groups”, Eur. J. Pharm. Sci. 4: 49-59 (1996); Gangwar et al., “Pro-drug, molecular structure and percutaneous delivery”, Des. Biopharm. Prop. Prodrugs Analogs, [Symp.] Meeting Date 1976, 409-21. (1977); Nathwani and Wood, “Penicillins: a current review of their clinical pharmacology and therapeutic use”, Drugs 45(6): 866-94 (1993); Sinhababu and Thakker, “Prodrugs of anticancer agents”, Adv. Drug Delivery Rev. 19(2): 241-273 (1996); Stella et al., “Prodrugs. Do they have advantages in clinical practice?”, Drugs 29(5): 455-73 (1985); Tan et al. “Development and optimization of anti-HIV nucleoside analogs and prodrugs: A review of their cellular pharmacology, structure-activity relationships and pharmacokinetics”, Adv. Drug Delivery Rev. 39(1-3): 117-151 (1999); Taylor, “Improved passive oral drug delivery via prodrugs”, Adv. Drug Delivery Rev., 19(2): 131-148 (1996); Valentino and Borchardt, “Prodrug strategies to enhance the intestinal absorption of peptides”, Drug Discovery Today 2(4): 148-155 (1997); Wiebe and Knaus, “Concepts for the design of anti-HIV nucleoside prodrugs for treating cephalic HIV infection”, Adv. Drug Delivery Rev.: 39(1-3):63-80 (1999); Waller et al., “Prodrugs”, Br. J. Clin. Pharmac. 28: 497-507 (1989), content of all of which is herein incorporated by reference in its entirety.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art.

The present invention furthermore includes all solvates of the compounds of a testosterone analogue or derivative or salt thereof, or an ornithine decarboxylase (ODC), for example hydrates, and the solvates formed with other solvents of crystallization, such as alcohols, ethers, ethyl acetate, dioxane, DMF, or a lower alkyl ketone, such as acetone, or mixtures thereof.

The present invention also includes prodrug forms of a testosterone analogue or derivative or salt thereof, or an ornithine decarboxylase (ODC), for example the alkyl esters of acids or any of the prodrugs for guanidines known to one skilled in the art. Thus, the present invention includes those compounds produced in vivo after administration of a different compound (or prodrug of the compound). The in vivo effects of compounds described herein, may not be exerted by those compounds as such, but by one or more degradation products.

Various polymorphs of compounds forming part of the present invention may be prepared by crystallization of a testosterone analogue or derivative or salt thereof, or an ornithine decarboxylase (ODC) under different conditions. Examples of different conditions are: using different commonly used solvents or their mixtures for crystallization; crystallization at different temperatures; and various modes of cooling, ranging from very fast to very slow cooling during crystallizations. Polymorphs may also be obtained by heating or melting the compound followed by gradual or fast cooling. The presence of polymorphs may be determined by IR spectroscopy, solid probe NMR spectroscopy, differential scanning calorimetry, powder X-ray diffraction or such other techniques.

The compounds of the present invention can have asymmetric centers at any of the carbon atoms, including any one of the R substituents. Consequently, compounds of a testosterone analogue or derivative or salt thereof, or an ornithine decarboxylase (ODC) can exist in enantiomeric or diastereomeric forms either in pure or substantially pure form or in mixtures thereof in all ratios. When diastereomeric or enantiomeric products are prepared, they can be separated by conventional methods for example, chromatographic or fractional crystallization. If mobile hydrogen atoms are present, the present invention also encompasses all tautomeric forms of the compounds of a testosterone analogue or derivative or salt thereof, or an ornithine decarboxylase (ODC).

The present invention is accordingly directed to a compound of a testosterone analogue or derivative or salt thereof, or an ornithine decarboxylase (ODC), or a prodrug thereof, or a pharmaceutically acceptable salt of the compound, for the manufacture of a medicament for the treatment of a mammal (e.g., human) having a disease or condition with low testosterone.

Another aspect of the present invention is directed to a method for preventing and/or minimizing the effect of low testosterone resulting from damage to the testis by administering to an affected mammal, (e.g., a female or male human), a therapeutically effective amount of a composition comprising testosterone, a testosterone analogue or derivative or salt thereof, in combination with an ornithine decarboxylase (ODC), or a prodrug thereof, or a pharmaceutically, acceptable salt thereof.

In n preferred embodiment of the invention the pharmaceutical composition as provides a dosage of 0.002 mg/kg to about 50 mg/kg to a subject.

In another embodiment of the invention the pharmaceutical composition reduces side effects associated with testosterone administration, such side effects comprise prostate related side effects in males and virilization in females. Said prostate related side effects include prostate cancer and enlarged prostate and virilization hirsutism and/or acne.

The pharmaceutical composition of the invention selectively promotes beneficial effects of testosterone, while preventing side effects related to testosterone administration by at least 10%.

In another embodiment of the invention the pharmaceutical composition reduces side effects associated with testosterone administration while promoting its beneficial effect like improvement in muscle mass and muscle performance, and physical function or measures of physical function in subjects with sarcopenia, frailty, or falls, or increased risk of falls. Said beneficial effects also comprise improved mobility in subjects with mobility limitation, treating muscle wasting and physical dysfunction in subjects who have muscle wasting due to bed rest, immobilization, surgery, or placement of an orthopedic cast or because of any acute or chronic illness and to promote recovery of physical function and strength and promote mobility after an acute or chornic illness, or hip fracture

In another embodiment of the invention the pharmaceutical composition may be a timed release or a sustained release or controlled release composition.

In another embodiment of the invention the pharmaceutical composition further comprises an additional agent. said agent is selected from the group consisting of leutropin, human horionic gonadotrophin, anti-cancer agent, anti-viral agent.

The composition of the invention can be administered in the form of gel, tablet, capsule, granulate, food product, troches, dispersions, suspensions solutions, or patches.

Characterizing Effect of Testosterone or Analogues Thereof in Combination with ODC Inhibitors

As disclosed herein, the combination of testosterone and an ODC inhibitor allows for selective inhibition of testosterone effects on the prostate without affecting the other anabolic roles of testosterone on the muscle and other organs, e.g., muscle growth.

In some embodiments, an ODC inhibitor as disclosed herein inhibits ODC enzymatic activity or expression by at least 10%, or about 20%, or about 25%, or about 50%, or about 75% or about 100%, or about 3-fold, or about 5-fold, or about 10-fold or more than 10-fold.

In some embodiments, the combination of testosterone and an ODC inhibitor as disclosed herein can be assessed in vivo in whole animal studies to demonstrate the utility the combination of an ODC inhibitor and testosterone or analogue or derivative thereof for elevating testosterone levels in the subject and having testosterone beneficial effects while preventing or reducing testosterone-associated prostate side effects with testosterone. Such in vivo studies also enable determination of appropriate pharmacokinetic properties determine whether testosterone and an ODC inhibitor as disclosed herein can reverse the age-related loss of testosterone production in men as a way of treating conditions associated with such testosterone reduction. In some embodiments, the combination of testosterone and at least one ODC inhibitor are assessed in C57BL/6 mice, and the combination of testosterone and an ODC inhibitor which increases testosterone levels in C57BL/6 and increases anabolic effects of testosterone by minimize testosterone-induced prostate growth can be selected for use in the methods, compositions and kits as disclosed herein.

In some embodiments, a combination of testosterone and an ODC inhibitor which increases testosterone levels in Mice by at least about 10% or more, or by about 15%, or at least about 20%, or at least about 30%, or at least about 40%, or at least about 50%, or at least about 60%, or at least about 70%, or at least about 80%, or at least about 90%, or at least about 100%, or at least about 1.5-fold, or at least about 2-fold, or at least about 3-fold or more than 3-fold, and prevents prostate growth or increase in prostate mass by at least 1%, or at least about 5% or at least about 6%, or at least about 7% or at least about 10%, or more than 10% as compared to the effect of testosterone in the absence of an ODC inhibitor can be selected for use in the methods, compositions and kits as disclosed herein.

In some embodiments, when the compounds as disclosed herein used for living beings other than humans, e.g., for agricultural or commercial animals, the combination of testosterone or analogue or salt thereof and an ODC inhibitor can be formulated to activate non-human testosterone and/or inhibit non human-ODC with an IC₅₀ at least an order of magnitude less than an IC₅₀ for inhibition of a human ODC, though more preferably at least two or three orders of magnitude less. In such an embodiment is useful for administering to non-human animals, such as agriculture animals, e.g., to increase testosterone in agriculture animals for meat production, but avoiding such compounds being also effective to the general population who are consumers of the meat, or getting into the water system from the agricultural plant.

The primary aspect of the present invention is a method for treating a mammal (e.g., human) having a disease or condition with low testosterone levels by administering a therapeutically effective amount of a combination of testosterone, analogue or salt thereof and an ODC inhibitor, or prodrugs thereof, or a pharmaceutically acceptable salt of the compound or of the prodrug, to the mammal.

The data obtained from cell culture assays and in vivo animal models can be used in formulating a range of dosage for use in humans. The dosage may vary depending upon the dosage form employed and the route of administration. For any compound used in the method of the invention, the therapeutically effective dose can be estimated initially from cell culture assays. A dose may be formulated in animal models to achieve a circulating plasma concentration range that includes the IC₅₀. Such information can be used to more accurately determine useful doses in humans. For example, levels of a combination of testosterone and an ODC inhibitor, or levels of testosterone in plasma may be measured by ordinary methods know to one skilled in the art, for example, by high performance liquid chromatography, or by other known methods in the art. Another aspect of the invention is directed to co-administrable combination of testosterone or analogue thereof in a first therapeutically effective amount, and an ornithine decarboxylase (ODC) inhibitor in a second therapeutically effective amount the combination selectively promotes beneficial effects of testosterone, while preventing side effects related to testosterone administration.

In an embodiment of the invention the co-administrable combination selectively promotes beneficial effects of testosterone, while preventing side effects related to testosterone administration by at least 10%.

The side effects related to testosterone administration comprise prostate related side effects in males and virilization in females. Prostate related side effects include prostate cancer and enlarged prostate and virilization includes hirsutism, and/oracne increased libido, male pattern muscle mass gain, increased risks of heart disease and hypertension.

The beneficial effects of testosterone may comprise improvement in muscle mass and muscle performance, and physical function or measures of physical function in subjects with sarcopenia, frailty, or falls, or increased risk of falls. Beneficial effects of testosterone also comprise improved mobility in subjects with mobility limitation, treating muscle wasting and physical dysfunction in subjects who have muscle wasting due to bed rest, immobilization, surgery, or placement of an orthopedic cast or because of any acute or chronic illness and to promote recovery of physical function and strength and promote mobility after an acute or chornic illness, or hip fracture.

### Method for Increasing Testosterone Levels in a Subject

Another aspect of the present invention is directed to a method for increasing testosterone levels in a subject comprising administration of testosterone or analogue thereof; and an ornithine decarboxylase (ODC) inhibitor, wherein said method selectively promotes beneficial effects of testosterone, while preventing side effects related to testosterone administration.

In an embodiment of the invention the testosterone or analogue thereof are administered in an amount of about 0.0002 mg/kg to about 50 mg/kg of body weight per day.

In an embodiment of the invention the side effects comprise prostate related side effects in males and virilization in females. Said prostate related side effects include prostate cancer and enlarged prostate and virilization includes hirsutism, and/or acne.

In an embodiment the beneficial effects comprise improvement in muscle mass and muscle performance, and physical function or measures of physical function in subjects with sarcopenia, frailty, or falls, or increased risk of falls. Beneficial effects also comprise improved mobility in subjects with mobility limitation, treating muscle wasting and physical dysfunction in subjects who have muscle wasting due to bed rest, immobilization, surgery, or placement of an orthopedic cast or because of any acute or chronic illness and to promote recovery of physical function and strength and promote mobility after an acute or chronic illness, or hip fracture

In an embodiment of the invention the method selectively promotes beneficial effects of testosterone, while preventing side effects related to testosterone administration by at least 10%.

An embodiment of the invention testosterone or analogue thereof and ornithine decarboxylase (ODC) inhibitor are administered simultaneously.

In another embodiment of the invention testosterone or analogue thereof and ornithine decarboxylase (ODC) inhibitor are administered sequentially.

in the present aspect invention the method is for increasing testosterone levels in a, wherein the subject the subject is a male or female. In another embodiment the subject has low testosterone levels. In yet another embodiment the subject has testosterone level of less than . . . 350 pg/ml. in yet another embodiment the subject suffers from hypogonadism. In another embodiment the subject has age-related low testosterone production. In another embodiment the subject suffers from a low-testosterone associated disorder. Said low testosterone associated disorder is type 2 diabetes, metabolic syndrome, dyslipidemia, obesity insulin resistance or hyperinsulinemia, male menopause, andropause, hypogonadotropichypogonadism, testicular failure, hyperprolactinemia, hypopituitarism, hypothalamic or pituitary disease, genetic diseases such as Klinefelter's syndrome, Kallman's syndrome, and Prader-Willi syndrome, testicular failure, infertility, myotonic dystrophy, acquired damage to the testes, such as alcoholism, physical injury, or viral diseases like mumps or testicular cancer.

In case of a female subject the female suffers from hirsutism, acne, hair loss, Another aspect of the present invention deals with method for treating a subject with low testosterone levels by selectively promoting beneficial effects of testosterone, while preventing side effects related to testosterone administration, comprising administration of; testosterone or analogue thereof; and an ornithine decarboxylase (ODC) inhibitor.

In specific embodiment of the invention the subject has testosterone level of less than 350 pg/ml. in another embodiment the subject has age-related low testosterone production. In yet another embodiment the subject suffers from a low-testosterone associated disorder selected from, but not limited to type 2 diabetes, metabolic syndrome, dyslipidemia, obesity insulin resistance or hyperinsulinemia, male menopause, andropause, hypogonadotropichypogonadism, testicular failure, hyperprolactinemia, hypopituitarism, hypothalamic or pituitary disease, genetic diseases such as Klinefelter's syndrome, Kallman's syndrome, and Prader-Willi syndrome, testicular failure, infertility, myotonic dystrophy, acquired damage to the testes, such as alcoholism, physical injury, or viral diseases like mumps or testicular cancer.

In specific embodiments the ornithine decarboxylase inhibitor to be used for the method of treatment of the present invention is selected from the group consisting of 2-difluoromethylomithine, N-(4′-Pyridoxyl)-Ornithine(BOC)-OMe(POB), α-methyl ornithine, 1,4-diamino-2-butanone (DAB), antizyme (AZ) or combination thereof.

In specific embodiments the testosterone analogue to be used for the method of treatment of the present invention is selected from the group consisting of testosterone ester, testosterone prodrugs, fatty acid ester of testosterone, or testosterone metabolites. In an preferred embodiment of the invention the testosterone metabolite is dihydrotestosterone. In another preferred embodiment the ester of testosterone is a C2-C13 alkyl ester, more preferably, an undecanoate acid ester of testosterone.

In an embodiment of the invention the testosterone or analogue thereof and ODC inhibitor are ach administered in an amount of about 0.0002 mg/kg to about 50 mg/kg of body weight per day.

In an embodiment the method of treatment of the present invention prevents side effects related to testosterone administrarion and comprise prostate related side effects in males and virilization in females. Prostate related side effects include prostate cancer and enlarged prostate and virilization includes hirsutism and acne.

In an embodiment of the invention the method of treatment prevents side associated with testosterone administration while promoting beneficial effects of testosterone which comprise improvement in muscle mass and muscle performance, and physical function or measures of physical function in subjects with sarcopenia, frailty, or falls, or increased risk of falls. Beneficial effects of testosterone also comprise improved mobility in subjects with mobility limitation associated with sarcopenia, muscle atrophy and weakness due to acute or chronic illness, bed rest, or fracture, treating muscle wasting and physical dysfunction in subjects who have muscle wasting due to bed rest, immobilization, surgery, or placement of an orthopedic cast or because of any acute or chronic illness and to promote recovery of physical function and strength and promote mobility after an acute or chornic illness, or hip fracture.

In an embodiment of the present invention the method of treatment selectively promotes beneficial effects of testosterone, while preventing side effects related to testosterone administration by at least 10%.

In an embodiment of the present invention the testosterone or analogue thereof and ornithine decarboxylase (ODC) inhibitor are administered simultaneously.

In another embodiment of the invention testosterone or analogue thereof and ornithine decarboxylase (ODC) inhibitor are administered sequentially.

In another embodiment of the present invention testosterone or analogue thereof; and an ornithine decarboxylase (ODC) inhibitor can be administered through oral, intravenous, intramuscular, transdermal, rectal, parenteral, intrathecal, vaginal routes or by direct absorption through mucous membrane.

In another specific embodiment of the invention the invention testosterone or analogue thereof; and an ornithine decarboxylase (ODC) inhibitor or their composition is administered in the form of gel, tablet, capsule, granulate, food product, troches, dispersions, suspensions solutions, or patches.

In another aspect of the invention provides a method for treating hypogonadism in males by selectively promoting beneficial effects of testosterone, while preventing prostate related side effects, comprising administration of; testosterone or analogue thereof; and an ornithine decarboxylase (ODC) inhibitor.

Another aspect of the present invention is directed to a diagnostic kit comprising a combination of testosterone or analogue thereof; and an ornithine decarboxylase (ODC) inhibitor. The diagnostic kit of the invention further comprises instructions for use thereof.

Administration of testosterone, analogue thereof or an ODC inhibitor or a composition comprising the combination of testosterone or analogue thereof and an ODC inhibitor as disclosed herein may be by oral, parenteral, sublingual, rectal, or enteral administration, or pulmonary absorption or topical application. Direct administration of a composition comprising a combination of testosterone, analogue or salt thereof and an ODC inhibitor as disclosed herein to a subject can be by oral, parenteral, sublingual, rectal such as suppository or enteral administration, or by pulmonary absorption or topical application.

Parenteral administration may be by intravenous (IV) injection, subcutaneous (s.c.) injection, intramuscular (i.m) injection, intra-arterial injection, intrathecal (i.t.) injection, intra-peritoneal (i.p) injection, or direct injection or other administration to the subject.

In addition to a composition comprising a combination of testosterone, analogue or salt thereof and an ODC inhibitor as disclosed herein, such compositions can optionally contain pharmaceutically-acceptable carriers and other ingredients known to facilitate administration and/or enhance uptake (e.g., saline, dimethyl sulfoxide, lipid, polymer, affinity-based cell specific-targeting systems). In some embodiments, a composition a composition comprising a combination of testosterone, analogue or salt thereof and an ODC inhibitor as disclosed herein and/or salts thereof can be incorporated in a gel, sponge, or other permeable matrix (e.g., formed as pellets or a disk) and placed in proximity to the endothelium for sustained, local release. In some embodiments, a composition comprising a composition comprising a combination of testosterone, analogue or salt thereof and an ODC inhibitor as disclosed herein and/or salts thereof can be administered in a single dose or in multiple doses which are administered at different times.

In some embodiments, the testosterone, or testosterone derivative or analogue or salt thereof, and ODC inhibitor, such as DFMO as cyclosporine as disclosed herein can be provided in a therapeutic composition so that the preferred amounts of the testosterone agent and the ODC inhibitor are supplied by a single dosage, for example a single capsule enabling testosterone, (or a testosterone derivative or analogue or salt thereof), and ODC inhibitor, such as DFMO to be administered to a subject at about the same time.

In some embodiments of the invention, testosterone, (or a testosterone derivative, analogue or salt thereof), and ODC inhibitor, such as DFMO, can be administered substantially simultaneously, meaning that both agents can be provided in a single dosage, for example by mixing the agents and incorporating the mixture into a single capsule or within a short time of each other. In alternative embodiments, testosterone, (or a testosterone derivative, analogue or salt thereof), and ODC inhibitor, such as DFMO can be administered substantially simultaneously by administration in separate dosages within a short time period, for example within one hour or less, 45 minutes or less, 30 minutes or less, 15 minutes or less, 10 minutes or less, 5 minutes or less and all time periods in between. Alternatively, testosterone, (or a testosterone derivative, analogue or salt thereof), and ODC inhibitor, such as DFMO can be administered sequentially, meaning that separate dosages, and possibly even separate dosage forms of testosterone, (or a testosterone derivative, analogue or salt thereof), and ODC inhibitor, such as DFMO can be administered at separate times, for example on a staggered schedule but with equal frequency of administration of each of the testosterone, (or a testosterone derivative, analogue or salt thereof), and ODC inhibitor, such as DFMO. Of course, it is also possible that testosterone, (or a testosterone derivative, analogue or salt thereof), can be administered either more or less frequently than the one or more ODC inhibitor. Different agents have different half lives, thus one can stagger schedules and still maintain both agents being effective in an individual. In any case, it is preferable that, among successive time periods of a sufficient length, for example one day, the weight ratio of testosterone, (or a testosterone derivative, analogue or salt thereof), are administered to the weight ratio of the ODC inhibitor administered remains constant.

In some embodiments, testosterone, (or a testosterone derivative, analogue or salt thereof), and ODC inhibitor, such as DFMO can be administered sequentially, for example the two agents can be administered within about 1 hour or more of each other, as long as they are both biologically active within the same time period. For example, the time between administration of the testosterone, (or a testosterone derivative, analogue or salt thereof), and the ODC inhibitor, such as DFMO can vary depending on the half life of each of the agents. For example a longer time period can occur between administration of testosterone, (or a testosterone derivative, analogue or salt thereof), and the ODC inhibitor, if both agents have long half lives, as compared to a shorter time period between administration of each agent if both agents have short half lives. Alternatively, the first agent administered for example testosterone, (or a testosterone derivative, analogue or salt thereof), can be administered in a time-release capsule to release the biologically active testosterone agent at a certain period after administration, or testosterone, (or a testosterone derivative, analogue or salt thereof), can be administered as a pro-drug that takes a certain time period to be metabolized to become the biologically active compound, and where in both situations the testosterone becomes biologically active at a time period which coincides with administration and biological activity of the ODC inhibitor, such as DFMO.

In alternative embodiments, testosterone, (or a testosterone derivative, analogue or salt thereof), and ODC inhibitor, such as DFMO can be administered sequentially, for example, one can administer a testosterone to a subject followed by at least one ODC inhibitor, then a testosterone agent, and so forth, so that the subject is administered, in an alternating regimen, doses of testosterone (or a testosterone derivative, analogue or salt thereof) followed by a dose of an ODC inhibitor, such as DFMO.

In alternative embodiments, a subject is administered one agent continuously and administered the other agent in repeated doses. By way of an example but not as a limitation, a subject can be continuously administered testosterone (or a testosterone derivative, analogue or salt thereof) by any suitable means such as a transdermal patch or other continuous administration method such as catheterization or by pump administration and administered an ODC inhibitor, such as DFMO at regular intervals, for example but not limited to daily, twice a day, twice a week, monthly etc by any suitable means known by persons of ordinary skill in the art and disclosed herein to keep the agents active in an individual.

In alternative embodiments, a subject is administered testosterone, (or a testosterone derivative, analogue or salt thereof), and ODC inhibitor, such as DFMO by pulse chase schedules. For example, a subject is administered one agent, such as ODC inhibitor for a brief period of time (the pulse) and then a subject is administered the other agent, such as an testosterone (or a testosterone derivative, analogue or salt thereof) for a longer period (the chase). In such embodiments, a subject can be administered varying amounts of each agent for each pulse-chase administration regimen. The pulse-chase regimine can be switched so that the subject is administered testosterone for limited period of time, followed by administration of the ODC inhibitor for a longer period of time.

In some embodiments, a subject is administered varying amounts of each agent, for example varying amounts of testosterone (or a testosterone derivative, analogue or salt thereof) and varying amounts of the ODC inhibitor, such as DFMO.

Daily dosages can vary within wide limits and will be adjusted to the subject requirements in each particular case. In general, for administration to adults, an appropriate daily dosage has been described above, although the limits that were identified as being preferred can be exceeded if necessary. The daily dosage can be administered as a single dosage or in divided dosages. Various delivery systems include capsules, tablets, and gelatin capsules, for example.

Any suitable route and any combination of routes of administration can be employed for providing a subject with an effective dosage of a combined therapy of the present invention. For example, oral, rectal, transdermal, parenteral (subcutaneous, intramuscular, intravenous), intrathecal, and like forms of administration can be employed. Dosage forms include tablets, troches, dispersions, suspensions, solutions, capsules, patches, and the like.

The composition of the present invention is administered and dosed in accordance with good medical practice, taking into account the clinical condition of the individual subject, the site and method of administration, scheduling of administration, subject age, sex, body weight and other factors known to medical practitioners.

In some embodiments, compositions can be directly or indirectly administered to a subject or patient. In some embodiments, indirect administration is performed, for example, by administering a composition comprising a combination of testosterone, analogue or salt thereof and an ODC inhibitor as disclosed herein to cells ex vivo and subsequently introducing the treated cells to the subject, e.g., human patient. Alternatively, the cells may be obtained from the patient to be treated or from a genetically related or unrelated patient. Related patients offer some advantage by lowering the immunogenic response to the cells to be introduced. For example, using techniques of antigen matching, immunologically compatible donors can be identified and utilized.

Alternatively, pharmaceutical compositions comprising a combination of testosterone, analogue or salt thereof and an ODC inhibitor as disclosed herein and/or salts thereof can be added to the culture medium of cells ex vivo.

Pharmaceutical compositions comprising a combination of testosterone, analogue or salt thereof and an ODC inhibitor as disclosed herein and/or salts thereof can be administered by any known route. By way of example, Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. and/or salts thereof can be administered by a mucosal, pulmonary, topical, or other localized or systemic route (e.g., enteral and parenteral).

The phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection, infusion and other injection or infusion techniques, without limitation. The phrases “systemic administration,” “administered systemically”, “peripheral administration” and “administered peripherally” as used herein mean the administration of the agents as disclosed herein such that it enters the animal's system and, thus, is subject to metabolism and other like processes, for example, subcutaneous administration.

Preparations for parenteral administration of a combination of testosterone, analogue or salt thereof and an ODC inhibitor as disclosed herein include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation, for example the carrier does not decrease the impact of the agent on the treatment. In other words, a carrier is pharmaceutically inert.

Preparations of a composition comprising a combination of testosterone, analogue or salt thereof and an ODC inhibitor are administered in effective amounts. An effective amount is that amount of a pharmaceutical preparation that alone, or together with further doses, stimulates the desired response. In the case of treating a disorder or condition that is associated with abnormal testosterone levels or activity and/or where it the desired response is increasing testosterone levels, and/or increasing testosterone production or reducing the onset, stage or progression of male menopause. This may involve only slowing the progression of decrease in age-related testosterone levels, or slowing the progression temporarily, although more preferably, it involves halting the progression of decreasing of the low testosterone levels permanently. An effective amount for treating a low testosterone disease or disorder is an amount that alters (e.g., increases) the amount of testosterone level in the subject with a low testosterone disease or disorder, with respect to that amount that would occur in the absence of the active compound.

In some embodiments of the invention relates to, in part, the administration of an effective amount of a composition comprising a combination of testosterone, analogue or salt thereof and an ODC inhibitor as disclosed herein. In some embodiments, the amount of testosterone or derivative or analogue or salt thereof is administered in effective amounts. Typically an effective amount of such testosterone compounds and the amount of an ODC inhibitor can be determined by an ordinary physician, or in clinical trials, establishing an effective dose for a test population versus a control population in a blind study, where the effective dose results in beneficial anabolic effects of testosterone (e.g., muscle growth) while minimizing the testosterone-induced increases in prostate growth and/or prostate-associated side effects.

In some embodiments, an effective amount is an amount that increases testosterone in a subject, or diminishes or eliminates a low testosterone disease or disorder in the subject. Thus, an effective amount is an amount of a combination of testosterone, analogue or salt thereof and an ODC inhibitor as disclosed herein, that when administered to a subject increases the absolute testosterone level in the serum of the subject as compared to without the administration of a combination of testosterone, analogue or salt thereof and an ODC inhibitor, and where the ODC inhibitor is at an effective dose to reduce testosterone-induced increase in prostate mass and/or prostate growth.

### Oral Formulations

In some embodiments, administration of a composition comprising a combination of testosterone, analogue or salt thereof and an ODC inhibitor as disclosed herein is in an oral formulations. Alternatively, in some embodiments, compositions comprising a combination of testosterone, analogue or salt thereof and an ODC inhibitor can also be administered to the nasal passages as a spray. Sprays also provide immediate access to the pulmonary system and are the preferable methods for administering compositions immediately to the subject. Access to the gastrointestinal tract is gained using oral, enema, or injectable forms of administration. For example, administration of the compositions comprising a combination of testosterone, analogue or salt thereof and an ODC inhibitor as disclosed herein and/or salts thereof to a subject is preferably oral. As a result, the subject can undergo administration of a composition comprising a combination of testosterone, analogue or salt thereof and an ODC inhibitor at home.

As indicated above, orally active compositions comprising a combination of testosterone, analogue or salt thereof and an ODC inhibitor as disclosed herein are preferred for at least a portion of the cycle of therapy, as oral administration is usually the safest, most convenient, and economical mode of drug delivery. Consequently, compositions as disclosed herein comprising a combination of testosterone, analogue or salt thereof and an ODC inhibitor as disclosed herein thereof can be modified to increase their oral bioavailability by reducing or eliminating their polarity. This can often be accomplished by formulating a composition with a complimentary reagent that neutralizes its polarity, or by modifying the compound with a neutralizing chemical group. Oral bioavailability is also a problem, because drugs are exposed to the extremes of gastric pH and gastric enzymes. Accordingly, problems associated with oral bioavailability can be overcome by modifying the molecular structure to be able to withstand very low pH conditions and resist the enzymes of the gastric mucosa such as by neutralizing an ionic group, by covalently bonding an ionic interaction, or by stabilizing or removing a disulfide bond or other relatively labile bond.

In some embodiments, an oral formulation of a composition comprising a combination of testosterone, analogue or salt thereof and an ODC inhibitor as disclosed herein can comprise trappsol and/or captisol for stability, or alternatively cyclodextrin. In some embodiments, an oral formulation a composition comprising a combination of testosterone, analogue or salt thereof and an ODC inhibitor as disclosed herein comprises a preservative, for example, methylparaben, which can be used for example at a concentration of about 0.25% for the syrup formulation in the pH range of 6-7. Applicants recommend a preservative challenge test to be conducted at a later stage and a variety of different timepoints to determine the optimal concentration of methylparaben based on the results of the preservative challenge test.

In some embodiments, an oral formulation of a composition comprising a combination of testosterone, analogue or salt thereof and an ODC inhibitor as disclosed herein thereof can also comprise a sweetener, for example, it can be formulated as a syrup using any sweetener commonly known to one of ordinary skill in the art, and in different combinations and percentage of the formulation. Exemplary sweeteners include, but are not limited to, Sucrose syrup, High Fructose Corn syrup, Sodium saccharin, Aspartame, Acesulfame and Sucralose. In some embodiments, an oral formulation of a combination of testosterone, analogue or salt thereof and an ODC inhibitor as disclosed herein comprises at least one sweetener(s) or a combination of any sweeteners and a stabilizer, e.g., but not limited to Trappsol.

In some embodiments, an oral formulation of a composition comprising a combination of testosterone, analogue or salt thereof and an ODC inhibitor as disclosed herein comprises at least one, or any combination of High Fructose Corn syrup, Sodium saccharin, Aspartame, Acesulfame or Sucralose. Without wishing to be bound by theory, High Fructose corn syrup was found to have a better taste-masking effect than Sucrose syrup. Sodium saccharin was found to impart greater initial sweetness than Aspartame but provided a very bitter after-taste. Acesulfame by itself provided a good initial sweetness with bitter after-taste but in combination with Sucralose provided a lingering sweet after-taste.

In some embodiments, an oral formulation of a composition comprising a combination of testosterone, analogue or salt thereof and an ODC inhibitor as disclosed herein can also comprise a flavor, for example, any flavor known to persons of ordinary skill in the art, for example, but without limitation, Chemy, Grape, Lemon, Pineapple, Orange, Menthol, Chocolate, Mint, Chocolate mint.

### Enteric Coated Formulation

In some embodiments, a composition comprising a combination of testosterone, analogue or salt thereof and an ODC inhibitor as disclosed herein can be formulated as tablets, for oral and/or enteral administration in accordance with conventional procedures employing solid carriers well-known in the art. Capsules employed for oral formulations to be used with the methods of the present invention can be made from any pharmaceutically acceptable material, such as gelatin or cellulose derivatives. Sustained release oral delivery systems and/or enteric coatings for orally administered dosage forms are also contemplated, such as those described in U.S. Pat. No. 4,704,295, “Enteric Film-Coating Compositions,” issued Nov. 3, 1987; U.S. Pat. No. 4,556,552, “Enteric Film-Coating Compositions,” issued Dec. 3, 1985; U.S. Pat. No. 4,309,404, “Sustained Release Pharmaceutical Compositions,” issued Jan. 5, 1982; and U.S. Pat. No. 4,309,406, “Sustained Release Pharmaceutical Compositions,” issued Jan. 5, 1982, which are all incorporated herein in their entirety by reference.

Accordingly, in some embodiments oral formulations of a composition comprising a combination of testosterone, analogue or salt thereof and an ODC inhibitor as disclosed herein can be in the form of a tablet formulation, for example, a tablet an enteric polymer casing. An example of such a preparation can be found in WO2005/021002, which is incorporated herein in its entirety by reference. The active material in the core can be present in a micronised or solubilized form. In addition to active materials the core can contain additives conventional to the art of compressed tablets. Appropriate additives in such a tablet can comprise diluents such as anhydrous lactose, lactose monohydrate, calcium carbonate, magnesium carbonate, dicalcium phosphate or mixtures thereof; binders such as microcrystalline cellulose, hydroxypropylmethylcellulose, hydroxypropyl-cellulose, polyvinylpyrrolidone, pre-gelatinised starch or gum acacia or mixtures thereof; disintegrants such as microcrystalline cellulose (fulfilling both binder and disintegrant functions) cross-linked polyvinylpyrrolidone, sodium starch glycollate, croscarmellose sodium or mixtures thereof; lubricants, such as magnesium stearate or stearic acid, glidants or flow aids, such as colloidal silica, talc or starch, and stabilizers such as desiccating amorphous silica, coloring agents, flavors etc. In some embodiments, a tablet comprises lactose as diluent. When a binder is present, it is preferably hydroxypropylmethyl cellulose. In some embodiments, a tablet comprises magnesium stearate as lubricant. In some embodiments, a tablet comprises croscarmellose sodium as disintegrant, or can comprise a microcrystalline cellulose.

Examples of solid carriers include starch, sugar, bentonite, silica, and other commonly used carriers. Further non-limiting examples of carriers and diluents that can be used in the formulations of the present invention include saline, syrup, dextrose, and water.

In some embodiments, a diluent can be present in a range of 10-80% by weight of the core. The lubricant can be present in a range of 0.25-2% by weight of the core. The disintegrant can be present in a range of 1-10% by weight of the core. Microcrystalline cellulose, if present, can be present in a range of 10-80% by weight of the core.

In some embodiments, the active ingredient, e.g., a combination of testosterone, analogue or salt thereof and an ODC inhibitor comprises between 10 and 50% of the weight of the core, more preferably between 15 and 35% of the weight of the core (calculated as free base equivalent). The core can contain any therapeutically suitable dosage level of the active ingredient e.g., a combination of testosterone, analogue or salt thereof and an ODC inhibitor, but preferably contains up to 150 mg as free base of the active ingredient. In some embodiments, the core contains 20, 30, 40, 50, 60, 80 or 100 mg as free base of the active ingredient. The active ingredient e.g., a combination of testosterone, analogue or salt thereof and an ODC inhibitor can be present as the free base, or as any pharmaceutically acceptable salt. If the active ingredient e.g., a combination of testosterone, analogue or salt thereof and an ODC inhibitor present as a salt, the weight is adjusted such that the tablet contains the desired amount of active ingredient, calculated as free base of the salt.

In some embodiments, the core can be made from a compacted mixture of its components. The components can be directly compressed, or can be granulated before compression. Such granules can be formed by a conventional granulating process as known in the art. In an alternative embodiment, the granules can be individually coated with an enteric casing, and then enclosed in a standard capsule casing.

In some embodiments, the core can be surrounded by a casing that comprises an enteric polymer. Examples of enteric polymers are cellulose acetate phthalate, cellulose acetate succinate, methylcellulose phthalate, ethylhydroxycellulose phthalate, polyvinylacetate pthalate, polyvinylbutyrate acetate, vinyl acetate-maleic anhydride copolymer, styrene-maleic mono-ester copolymer, methyl acrylate-methacrylic acid copolymer or methacrylate-methacrylic acid-octyl acrylate copolymer. These can be used either alone or in combination, or together with other polymers than those mentioned above. The casing can also include insoluble substances which are neither decomposed nor solubilised in living bodies, such as alkyl cellulose derivatives such as ethyl cellulose, crosslinked polymers such as styrene-divinylbenzene copolymer, polysaccharides having hydroxyl groups such as dextran, cellulose derivatives which are treated with bifunctional crosslinking agents such as epichlorohydrin, dichlorohydrin or 1,2-, 3,4-diepoxybutane. The casing can also include starch and/or dextrin.

In some embodiments, enteric coating materials are the commercially available EUDRAGIT® enteric polymers such as EUDRAGIT® L, EUDRAGIT® S and EUDRAGIT® NE, used alone or with a plasticiser. Such coatings are normally applied using a liquid medium, and the nature of the plasticiser depends upon whether the medium is aqueous or non-aqueous. Plasticisers for use with aqueous medium include propylene glycol, triethyl citrate, acetyl triethyl citrate or CITROFLEX® or CITROFLEX® A2. Non-aqueous plasticisers include these, and also diethyl and dibutyl phthalate and dibutyl sebacate. A preferred plasticiser is Triethyl citrate. The quantity of plasticiser included will be apparent to those skilled in the art.

In some embodiments, a casing can also include an anti-tack agent such as talc, silica or glyceryl monostearate. In some embodiments, an anti-tack agent is glyceryl monostearate. Typically, the casing can include around 5-25 wt % Plasticiser and up to around 50 wt % of anti tack agent, preferably 1-10 wt % of anti-tack agent.

If desired, a surfactant can be included to aid with forming an aqueous suspension of the polymer. Many examples of possible surfactants are known to the person skilled in the art. Preferred examples of surfactants are polysorbate 80, polysorbate 20, or sodium lauryl sulphate. If present, a surfactant can form 0.1-10% of the casing, preferably 0.2-5% and particularly preferably 0.5-2%

In one embodiment, there is a seal coat included between the core and the enteric coating. A seal coat is a coating material that can be used to protect the enteric casing from possible chemical attack by any alkaline ingredients in the core. The seal coat can also provide a smoother surface, thereby allowing easier attachment of the enteric casing. A person skilled in the art would be aware of suitable coatings. Preferably the seal coat is made of an OPADRY coating, and particularly preferably it is Opadry White OY-S-28876.

In some embodiments, an example of an enteric-coated formulation as described in WO2005/021002, can comprise varying amounts of one or more of testosterone, or analogue or salt thereof and an ODC inhibitor. In that example, lactose monohydrate, microcrystalline cellulose, the active ingredient, the hydroxypropyl methyl cellulose and half of the croscarmellose sodium were screened into a 10 Litre Fielder high-shear blender (any suitable high shear blender could be used) and blended for 5 minutes at 300 rpm with the chopper off. The mixture was then granulated by the addition of about 750 ml water whilst continuing to blend. The granules were dried in a Glatt 3/5 fluid bed drier, screened by Comil into a Pharmatec 5 Liter bin blender and then blended with any lactose anhydrous given in the formula plus the remainder of the croscarmellose sodium over 5 minutes at 20 rpm. Magnesium stearate was screened into the blender and the mixing process continued for a further 1 minute at 10 rpm. The lubricated mix was compressed using a Riva Piccolla rotary tablet press fitted with 9.5 mm round normal convex punches (any suitable tablet press could be used). The sealcoat, and subsequently the enteric coat, are applied by spraying of an aqueous suspension of the coat ingredients in a Manesty 10 coater using parameters for the coating process as recommended by the manufacturers of the coating polymers (again, any suitable coater could be used).

Other enteric-coated preparations of this sort can be prepared by one skilled in the art, using these materials or their equivalents.

### Other Formulations and Routes of Administration

In some embodiments, the invention provides a composition comprising a combination of testosterone, or analogue or salt thereof and an ODC inhibitor as disclosed herein for use as a medicament, methods for preparing the medicament and methods for the sustained release of the medicament in vivo. Delivery systems can include time-release, delayed release or sustained release delivery systems, or as a pro-drug composition. Such systems can avoid repeated administrations of a pharmaceutical composition comprising a combination of testosterone, or analogue or salt thereof and an ODC inhibitor to increase convenience to the subject and/or the physician.

Many types of release delivery systems are available and known to those of ordinary skill in the art. They include, but are not limited to, polymer-based systems such as polylactic and polyglycolic acid, poly(lactide-glycolide), copolyoxalates, polyanhydrides, polyesteramides, polyorthoesters, polyhydroxybutyric acid, and polycaprolactone. Microcapsules of the foregoing polymers containing drugs are described in, for example, U.S. Pat. No. 5,075,109. Nonpolymer systems that are lipids including sterols such as cholesterol, cholesterol esters and fatty acids or neutral fats such as mono-, di- and tri-glycerides; phospholipids; hydrogel release systems; silastic systems; peptide based systems; wax coatings, compressed tablets using conventional binders and excipients, partially fused implants and the like. Specific examples include, but are not limited to: (a) erosional systems in which the polysaccharide is contained in a form within a matrix, found in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

In one embodiment, a vehicle is a biocompatible microparticle or implant that is suitable for implantation into the mammalian recipient. Exemplary bioerodible implants that are useful in accordance with this method are described in PCT International application no. WO 95/24929, entitled “Polymeric Gene Delivery System”, describes a biocompatible, preferably biodegradable polymeric matrix for containing an exogenous gene under the control of an appropriate promoter. The polymeric matrix is used to achieve sustained release of the exogenous gene in the patient. In accordance with the instant invention, the compound(s) of the invention is encapsulated or dispersed within the biocompatible, preferably biodegradable polymeric matrix disclosed in WO 95/24929. The polymeric matrix preferably is in the form of a microparticle such as a microsphere (wherein the compound is dispersed throughout a solid polymeric matrix) or a microcapsule (wherein the compound is stored in the core of a polymeric shell).

Other forms of the polymeric matrix for containing the compounds of the invention include films, coatings, gels, implants, and stents. The size and composition of the polymeric matrix device is selected to result in favorable release kinetics in the tissue into which the matrix device is implanted. The size of the polymeric matrix device further is selected according to the method of delivery that is to be used. The polymeric matrix composition can be selected to have both favorable degradation rates and also to be formed of a material that is bioadhesive, to further increase the effectiveness of transfer when the device is administered to a vascular surface. The matrix composition also can be selected not to degrade, but rather, to release by diffusion over an extended period of time.

Both non-biodegradable and biodegradable polymeric matrices can be used to deliver agents and compounds of the invention of the invention to the subject. Biodegradable matrices are preferred. Such polymers may be natural or synthetic polymers. Synthetic polymers are preferred. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multi-valent ions or other polymers.

In general, compositions comprising a combination of testosterone, or analogue or salt thereof and an ODC inhibitor as disclosed herein can be delivered using the bioerodible implant by way of diffusion, or more preferably, by degradation of the polymeric matrix. Exemplary synthetic polymers which can be used to form the biodegradable delivery system include: polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene and polyvinylpyrrolidone.

Examples of non-biodegradable polymers include ethylene vinyl acetate, poly(meth)acrylic acid, polyamides, copolymers and mixtures thereof. Examples of biodegradable polymers include synthetic polymers such as polymers of lactic acid and glycolic acid, polyanhydrides, poly(ortho)esters, polyurethanes, poly(butic acid), poly(valeric acid), and poly(lactide-cocaprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

Bioadhesive polymers of particular interest include bioerodible hydrogels may include, but are not limited to: polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

Use of a long-term sustained release implant comprising a composition comprising a combination of testosterone, or analogue or salt thereof and an ODC inhibitor as disclosed herein can be particularly suitable for treatment of subjects with an established low testosterone disease or disorder, as well as subjects at risk of developing a such a disease or disorder, or subjects who have prostate cancer or are in need to testosterone replacement therapy and are at risk of prostate cancer.

The “long-term” release, as used herein, means that the implant is constructed and arranged to deliver therapeutic levels of composition comprising a combination of testosterone, or analogue or salt thereof and an ODC inhibitor as disclosed herein for at least about 7 days, and in some embodiments about 30-60 days, and in some embodiments, for 4-6 months, or for 6-12 months, or longer than 12 months, for example, several years. In some embodiments, an implant may be positioned at or near the site of the testis, but alternatively, can be positioned anywhere in the subject where the compounds are delivered to the systemic system or for physiological function to increase testosterone levels in the subject yet reduce testosterone-induced prostate growth. In some embodiments, a composition comprising testosterone can be located anywhere in the body where the beneficial effect of testosterone is desired (e.g., muscle cells) and a composition comprising an ODC inhibitor can be located in close proximity to, or near the prostate to localize the inhibition of ODC to the prostate. Long-term release implants can also be positioned near male reproductive organs to allow regional administration of a combination of testosterone, or analogue or salt thereof and an ODC inhibitor. Long-term sustained release implants are well known to those of ordinary skill in the art and include some of the release systems described above.

In some embodiments, the compositions as disclosed herein is administered to a subject using an infusion pump (to infuse, for example, the compositions as disclosed herein into the subject's circulatory system) is generally used intravenously, although subcutaneous, arterial, and epidural infusions are occasionally used. Injectable forms of administration are sometimes preferred for maximal effect. When long-term administration by injection is necessary, medi-ports, in-dwelling catheters, or automatic pumping mechanisms are also preferred, wherein direct and immediate access is provided to the arteries in and around the heart and other major organs and organ systems.

In some embodiments, compositions as disclosed herein comprising a combination of testosterone, or an analogue, derivative thereof and a ODC inhibitor can be administered to a specific site may be by transdermal transfusion, such as with a transdermal patch, by direct contact to the cells or tissue, if accessible, or by administration to an internal site through an incision or some other artificial opening into the body.

### Doses and Administration Regimens

Suitable choices in amounts and timing of doses, formulation, and routes of administration of a combination of testosterone, or an analogue, derivative thereof with a ODC inhibitor as disclosed herein can be made with the goals of achieving a favorable response in the subject with low testosterone level, e.g., where a favorable response is an increase in the testosterone level in the subject by at least about 10% as compared to in the absence of such a compound, and avoiding undue toxicity or other harm thereto (i.e., safety), in particular, avoiding increase in prostate mass or growth or prostate-associated side effects. Therefore, “effective” refers to such choices that involve routine manipulation of conditions to achieve a desired effect.

A bolus of the formulation of a combination of testosterone, or an analogue, derivative thereof with a ODC inhibitor as disclosed herein can be administered to a subject over a short time period, for example, once a day is a convenient dosing schedule. Alternatively, an effective daily dose can be divided into multiple doses for purposes of administration, for example, two to twelve doses per day. Dosage levels of active ingredients in a pharmaceutical composition comprising a combination of testosterone, or an analogue, derivative thereof with a ODC inhibitor as disclosed herein can also be varied so as to achieve a transient or sustained concentration of the compound or derivative thereof in an individual, especially in and around the blood circulation and to result in the desired therapeutic response or protection. But it is also within the skill of the art to start doses at levels lower than required to achieve the desired therapeutic effect and to gradually increase the dosage until the desired effect is achieved.

In some embodiments, the amount of testosterone, or an analogue, derivative thereof with a ODC inhibitor as disclosed herein can be administered is dependent upon factors known to a person skilled in the art such as bioactivity and bioavailability of the compound (e.g., half-life in the body, stability, and metabolism); chemical properties of the compound (e.g., molecular weight, hydrophobicity, and solubility); route and scheduling of administration, and the like. It will also be understood that the specific dose level to be achieved for any particular individual can depend on a variety of factors, including age, gender, health, medical history, weight, combination with one or more other drugs, and severity of disease.

Production of a composition comprising a combination of testosterone, or an analogue, derivative thereof with a ODC inhibitor as disclosed herein according to present regulations will be regulated for good laboratory practices (GLP) and good manufacturing practices (GMP) by governmental agencies (e.g., U.S. Food and Drug Administration). This requires accurate and complete record keeping, as well as monitoring of QA/QC. Oversight of patient protocols by agencies and institutional panels is also envisioned to ensure that informed consent is obtained; safety, bioactivity, appropriate dosage, and efficacy of products are studied in phases; results are statistically significant; and ethical guidelines are followed. Similar oversight of protocols using animal models, as well as the use of toxic chemicals, and compliance with regulations is required.

Dosages, formulations, dosage volumes, regimens, and methods for analyzing results of increased the production of testosterone and levels of testosterone in a subject can vary. Thus, minimum and maximum effective dosages of a combination of testosterone, or an analogue, derivative thereof with a ODC inhibitor as disclosed herein vary depending on the method of administration. Increase in testosterone levels in a subject can occur within a specific dosage range, which varies depending on, for example, the race, sex, gender, age, and overall health of the subject receiving the dosage, the route of administration, whether a composition comprising a combination of testosterone, or an analogue, derivative thereof with a ODC inhibitor as disclosed herein is administered in conjunction with other molecules, and the specific regimen of administration. For example, in general, nasal administration requires a smaller dosage than oral, enteral, rectal, or vaginal (if being administered to female) administration.

In some embodiments, compositions of a combination of testosterone, or an analogue, derivative thereof with an ODC inhibitor as disclosed herein are also safe at effective dosages. Safe compositions are compositions that are not substantially toxic (e.g. cytotoxic or myelotoxic), or mutagenic at required dosages, do not cause adverse reactions or side effects, and are well-tolerated. Although side effects may occur, compositions are substantially safe if the benefits achieved from their use outweigh disadvantages that may be attributable to side effects. Unwanted side effects may include, but may not occur, frequent and/or sustained erections, nausea, vomiting, aggression, muscle development, baldness, hypersensitivity, allergic reactions, cardiovascular problems and other problems.

Compositions comprising a combination of testosterone, or an analogue, derivative thereof with a ODC inhibitor as disclosed herein useful for treating low testosterone levels do not substantially affect the viability of a Leydig cells or other cells in the testis, nor affect the effect of prostate function (e.g., do not increase prostate growth as compared to the treatment of testosterone in the absence of an ODC inhibitor).

Useful combination therapies will be understood and appreciated by those of skill in the art. Potential advantages of such combination therapies include the ability to use less of each of the individual active ingredients to minimize toxic side effects, synergistic improvements in efficacy, improved ease of administration or use, and/or reduced overall expense of compound preparation or formulation.

Administration of the composition comprising a combination of testosterone, or an analogue, derivative thereof with a ODC inhibitor as disclosed herein to a subject according to a method of the invention may be for prophylaxis, or alternatively, for therapeutic treatment of a subject diagnosed with low testosterone and/or a disorder associated with low testosterone as disclosed herein.

In some embodiments, the combination of testosterone, or an analogue, derivative thereof with a ODC inhibitor as disclosed herein can be administered to an adult, an adolescent, a child, in some embodiments, although rarely, the subject can be a neonate, an infant or in utero.

In some embodiments, the combination of testosterone, or an analogue, derivative thereof with a ODC inhibitor as disclosed herein can be administered according to a specific dosing regimen, e.g., in a single or multiple doses, or continuous or sporadic, or as deemed necessary based on an administration regime as determined by measuring total testosterone levels and/or free testosterone levels in the subject as disclosed herein.

In some embodiments, a combination of testosterone, or an analogue, derivative thereof with a ODC inhibitor as disclosed herein can be administered to a subject via a continuous infusion throughout the cycle of therapy. Alternatively, a combination of testosterone, or an analogue, derivative thereof with a ODC inhibitor as disclosed herein can be administered to a the subject over a single span of a few to several hours per day every day throughout the first period of the cycle of therapy.

Alternatively, in some embodiments a combination of testosterone, or an analogue, derivative thereof with a ODC inhibitor as disclosed herein can be administered to a subject in a single parenteral bolus, or orally, daily for several days throughout the treatment regimen or cycle, or weekly.

In some embodiments, a combination of testosterone, or an analogue, derivative thereof with a ODC inhibitor as disclosed herein can be administered alone, in combination with each other, and/or in combination with other drug therapies that are administered to subjects with a low-testosterone associated disease or disorders, such as sexual dysfunction, male menopause, as well as a subject with prostate cancer or at risk of prostate cancer and the like.

In some embodiments, a combination of testosterone, or an analogue, derivative thereof with at least one ODC inhibitor as disclosed herein can be administered alone or in combination with other suitable therapeutic agents useful in treating immune and inflammatory disorders such as immunosuppressants such as cyclosporins.

In some embodiments, a combination of testosterone, or an analogue, derivative thereof with at least one ODC inhibitor as disclosed herein are used in combination with other agents. For example, a combination of testosterone, or an analogue, derivative thereof with at least one ODC inhibitor as disclosed herein can be administered with an additional agent, e.g., lutropin (leutinizing hormone) or human horionic gonadotrophin that produce the cAMP response in Leydig cells. In some embodiments, an additional therapy is an anti-cancer treatment. Such an anti-cancer agent can be an agent that decreases growth of tumor after the anti-cancer effects of other therapies have decreased. The additional agent or therapy can also be another anti-viral or anti-cancer agent or therapy.

In some embodiments, additional compounds and drug therapies can be administered to a subject in combination (e.g., concurrent with, or after or before) administration of the a combination of testosterone, or an analogue, derivative thereof with at least one ODC inhibitor as disclosed herein as disclosed herein. Additional compounds and alternative drug therapies are well known to those of ordinary skill in the art and are administered by modes known to those of skill in the art. The drug therapies can be administered in amounts that are effective to achieve the physiological goals (to reduce a symptom from a low testosterone disease or disorder in a subject, e.g. low testosterone levels), in combination with at least one pharmaceutical compound of the invention e.g., testosterone, or an analogue, derivative thereof, or at least one ODC inhibitor.

In some embodiments, a combination of testosterone, or an analogue, derivative thereof with at least one ODC inhibitor as disclosed herein can be used in prophylaxis treatment, for example, where the subject has been diagnosed with low testosterone levels or likely to develop low testosterone levels (e.g., as a result of injury or damage to Leydig cells or the testis, for example, if the subject has, or will undergo treatment for testicular cancer such as for example, testis removal, chemotherapy or radiation therapy for the treatment of testicular cancer, a subject can be administered a combination of testosterone, or an analogue, derivative thereof with at least one ODC inhibitor as disclosed herein prior to, or concurrent with or subsequent to, the chemotherapy or radiation therapy, in order to prevent development of low testosterone levels which typically occur as a side-effect of the chemotherapy or radiation therapy for testicular cancer treatment, or as a result of damage to the testis.

In some embodiments, a combination of testosterone, or an analogue, derivative thereof with at least one ODC inhibitor as disclosed herein can be administered to a subject to augment the treatment of prostate and/or testicular cancer, for example, where a subject is undergoing, or has undergone, or will undergo conventional prostate and/or testicular cancer treatment, for example, chemotherapy, radiation therapy, antibody therapy, and/or other forms of prostate or testicular cancer therapy. Some conventional chemotherapeutic agents that would be useful in combination therapy with the methods and compositions of the invention where a combination of testosterone, or an analogue, derivative thereof with at least one ODC inhibitor are administered to a subject in combination with one or more anti-cancer therapies, such as but not limited to cyclophosphamides such as alkylating agents, the purine and pyrimidine analogs such as mercaptopurine, the vinca and vinca-like alkaloids, the etoposides or etoposide-like drugs, the antibiotics such as deoxyrubocin and bleomycin, the corticosteroids, the mutagens such as the nitrosoureas, antimetabolites including methotrexate, the platinum based cytotoxic drugs, the hormonal antagonists such as anti-insulin and anti-androgen, the anti-estrogens such as tamoxifen, and other agents such as doxorubicin, L-asparaginase, DTIC, mAMSA, procarbazine, hexamethylmelamine, and mitoxantrone. These agents could be given simultaneously, or alternately as defined by a protocol in combination with testosterone, or an analogue, derivative thereof and at least one ODC inhibitor as disclosed herein to a subject designed to maximize effectiveness, but minimize toxicity to the patient's body.

In some embodiments, a combination of testosterone, or an analogue, derivative thereof with at least one ODC inhibitor as disclosed herein can be prepared in solution as a dispersion, mixture, liquid, spray, capsule, or as a dry solid such as a powder or pill, as appropriate or desired. Solid forms may be processed into tablets or capsules or mixed or dissolved with a liquid such as water, alcohol, saline or other salt solutions, glycerol, saccharides or polysaccharide, oil, or a relatively inert solid or liquid. Liquids, pills, capsules or tablets administered orally may also include flavoring agents to increase palatability. Additionally, in some embodiments, a composition comprising a combination of testosterone, or an analogue, derivative thereof with at least one ODC inhibitor as disclosed herein can further comprise agents to increase shelf-life, such as preservatives, anti-oxidants, and other components necessary and suitable for manufacture and distribution of the composition. Compositions comprising a combination of testosterone, or an analogue, derivative thereof with at least one ODC inhibitor as disclosed herein can further comprise a pharmaceutically acceptable carrier or excipient. Carriers are chemical or multi-chemical compounds that do not significantly alter or affect the active ingredients of the compositions. Examples include water, alcohols such as glycerol and polyethylene glycol, glycerin, oils, salts such as sodium, potassium, magnesium, and ammonium, fatty acids, saccharides, or polysaccharides. Carriers may be single substances or chemical or physical combinations of these substances.

In some embodiments, a combination of testosterone, or an analogue, derivative thereof with at least one ODC inhibitor as disclosed herein can be used in combination with other agents to maximize the effect of the compositions administered in an additive or synergistic manner. Accordingly, a combination of testosterone, or an analogue, derivative thereof with at least one ODC inhibitor as disclosed herein can also comprise proteinaceous agents such as growth factors and/or cytokines. Such proteinaceous agents may also be aminated, glycosylated, acylated, neutralized, phosphorylated, or otherwise derivatized to form compositions that are more suitable for the method of administration to the patient or for increased stability during shipping or storage.

Compositions as disclosed herein comprising a combination of testosterone, or an analogue, derivative thereof with at least one ODC inhibitor as disclosed herein can be physiologically stable at therapeutically effective concentrations. Physiological stable compounds of a combination of testosterone, or an analogue, derivative thereof with at least one ODC inhibitor as disclosed herein not break down or otherwise become ineffective upon administration to a subject or prior to having a desired effect. A combination of testosterone, or an analogue, derivative thereof with at least one ODC inhibitor as disclosed herein can be structurally resistant to catabolism, and, thus, physiologically stable, or coupled by electrostatic or covalent bonds to specific reagents to increase physiological stability. Such reagents include amino acids such as arginine, glycine, alanine, asparagine, glutamine, histidine, or lysine, nucleic acids including nucleosides or nucleotides, or substituents such as carbohydrates, saccharides and polysaccharides, lipids, fatty acids, proteins, or protein fragments. Useful coupling partners include, for example, glycol, such as polyethylene glycol, glucose, glycerol, glycerin, and other related substances.

Physiological stability of a combination of testosterone, or an analogue, derivative thereof with at least one ODC inhibitor as disclosed herein can be measured from a number of parameters such as the half-life of the testosterone or analogue or derivative thereof, or the ODC inhibitor, or the half-life of active metabolic products derived therefrom. In some embodiments, a combination of testosterone, or an analogue, derivative thereof with at least one ODC inhibitor as disclosed herein have in vivo half-lives of greater than about 1 day, or 2 days, or 3 days, or 5 days, or about 7 days, or longer than about 7 days. A combination of testosterone, or an analogue, derivative thereof with at least one ODC inhibitor as disclosed herein is stable using this criteria, however, physiological stability can also be measured by observing the duration of biological effects on the patient. Preferably, a combination of testosterone, or an analogue, derivative thereof with at least one ODC inhibitor as disclosed herein is a stable composition where it has an in vivo half-life of greater than about 24 hours, a serum half-life of greater than about 24 hours, or a biological effect which continues for greater than 24 hours after treatment has been terminated or the serum level of the compound has decreased by more than half.

Preferably, a combination of testosterone, or an analogue, derivative thereof with at least one ODC inhibitor as disclosed herein are also not significantly biotransformed, degraded, or excreted by catabolic processes associated with metabolism. Although there may be some biotransformation, degradation, or excretion, these functions are not significant, if the composition is able to exert its desired effect.

In some embodiments, a combination of testosterone, or an analogue, derivative thereof with at least one ODC inhibitor as disclosed herein can additionally comprise chemicals that are substantially non-toxic. Substantially non-toxic means that the composition, although possibly possessing some degree of toxicity, is not harmful to the long-term health of the subject. Although the active component of the composition may not be toxic at the required levels, there may also be problems associated with administering the necessary volume or amount of the final form of the composition to the patient. For example, if a combination of testosterone, or an analogue, derivative thereof and at least one ODC inhibitor contains a salt, although the active ingredient may be at a concentration that is safe and effective, there can be a harmful build-up of sodium, potassium, or another ion. With a reduced requirement for the composition or at least the active component of that composition, the likelihood of such problems can be reduced or even eliminated. Consequently, although subjects may suffer minor or short term detrimental side-effects, the advantages of taking the composition outweigh the negative consequences.

The amount of a combination of testosterone, or an analogue, derivative thereof with at least one ODC inhibitor as disclosed herein that can be combined with a carrier material to produce a single dosage form will generally be that amount of the compound that produces a therapeutic effect. Generally out of one hundred percent, this amount will range from about 0.01% to 99% of the compound, preferably from about 5% to about 70%, most preferably from 10% to about 30%.

The data obtained from the cell culture assays and animal studies can be used in formulating a range of dosage for use in humans. The dosage of such compounds lies preferably within a range of circulating concentrations that include the ED₅₀ (the dose therapeutically effective in 50% of the population) with little or no toxicity. The dosage may vary within this range depending upon the dosage form employed and the route of administration utilized.

The therapeutically effective dose can be estimated initially from cell culture assays. Alternatively, a dose may be formulated in animal models of low testosterone levels, e.g., a Mice to achieve a circulating plasma concentration range that includes the IC₅₀ (i.e., the concentration of the therapeutic which achieves a half-maximal inhibition of symptoms) as determined in cell culture. Levels of the compound and/or testosterone levels in plasma may be measured, for example, by high performance liquid chromatography or other methods commonly known to persons in the art and disclosed herein. The effects of any particular dosage of a combination of testosterone, or an analogue, derivative thereof with at least one ODC inhibitor as disclosed herein can be monitored by a suitable bioassay.

The combination of testosterone, or an analogue, derivative thereof with at least one ODC inhibitor as disclosed herein as disclosed herein may be adjusted by the individual physician or veterinarian, particularly in the event of any complication. A therapeutically effective amount typically varies from 0.01 mg/kg to about 1000 mg/kg, preferably from about 0.1 mg/kg to about 200 mg/kg, and most preferably from about 0.2 mg/kg to about 20 mg/kg, in one or more dose administrations daily, for one or more days. It will be recognized by those of skill in the art that testosterone, or an analogue, derivative thereof may have detrimental effects at high amounts. Thus, an effective amount for use in the methods of the invention may be optimized such that the amount administered results in minimal negative side effects and maximum increase in safe and effective serum testosterone levels in the subject.

The absolute amount of a combination of testosterone, or an analogue, derivative thereof with at least one ODC inhibitor as disclosed herein will depend upon a variety of factors, including the type of testosterone (e.g., salt, derivative, analogue thereof) or type of ODC inhibitor used, the material selected for administration, whether the administration is in single or multiple doses, and individual subject parameters including age, physical condition, size, weight, and the stage of the disease or disorder. These factors are well known to those of ordinary skill in the art and can be addressed with no more than routine experimentation.

Accordingly, the dosage of a combination of testosterone, or an analogue, derivative thereof with at least one ODC inhibitor as disclosed herein can be determined by a physician and adjusted, as necessary, to suit observed effects of the treatment (e.g., an increase in serum testosterone levels while preventing an increase in prostate mass or prostate growth of more than 40% as compared to in the absence of an ODC inhibitor). Generally, the compositions are administered so that testosterone, or an analogue, derivative thereof or the ODC inhibitor as disclosed herein are given each at a dose from 1 μg/kg to 150 mg/kg, 1 μg/kg to 100 mg/kg, 1 μg/kg to 50 mg/kg, 1 μg/kg to 20 mg/kg, 1 μg/kg to 10 mg/kg, 1 μg/kg to 1 mg/kg, 100 μg/kg to 100 mg/kg, 100 μg/kg to 50 mg/kg, 100 μg/kg to 20 mg/kg, 100 μg/kg to 10 mg/kg, 100 μg/kg to 1 mg/kg, 1 mg/kg to 100 mg/kg, 1 mg/kg to 50 mg/kg, 1 mg/kg to 20 mg/kg, 1 mg/kg to 10 mg/kg, 10 mg/kg to 100 mg/kg, 10 mg/kg to 50 mg/kg, or 10 mg/kg to 20 mg/kg. It is to be understood that ranges given here include all intermediate ranges, for example, the range 1 mg/kg to 10 mg/kg includes 1 mg/kg to 2 mg/kg, 1 mg/kg to 3 mg/kg, 1 mg/kg to 4 mg/kg, 1 mg/kg to 5 mg/kg, 1 mg/kg to 6 mg/kg, 1 mg/kg to 7 mg/kg, 1 mg/kg to 8 mg/kg, 1 mg/kg to 9 mg/kg, 2 mg/kg to 10 mg/kg, 3 mg/kg to 10 mg/kg, 4 mg/kg to 10 mg/kg, 5 mg/kg to 10 mg/kg, 6 mg/kg to 10 mg/kg, 7 mg/kg to 10 mg/kg, 8 mg/kg to 10 mg/kg, 9 mg/kg to 10 mg/kg, and the like. It is to be further understood that the ranges intermediate to the given above are also within the scope of this invention, for example, in the range 1 mg/kg to 10 mg/kg, dose ranges such as 2 mg/kg to 8 mg/kg, 3 mg/kg to 7 mg/kg, 4 mg/kg to 6 mg/kg, and the like.

In some embodiments, a combination of testosterone, or an analogue, derivative thereof with at least one ODC inhibitor as disclosed herein are administered at a dosage so that testosterone or the ODC inhibitor has an in vivo, e.g., serum or blood, concentration of less than 500 nM, less than 400 nM, less than 300 nM, less than 250 nM, less than 200 nM, less than 150 nM, less than 100 nM, less than 50 nM, less than 25 nM, less than 20 nM, less than 10 nM, less than 5 nM, less than 1 nM, less than 0.5 nM, less than 0.1 nM, less than 0.05 nM, less than 0.01 nM, less than 0.005 nM, or less than 0.001 nM after 15 mins, 30 mins, 1 hr, 1.5 hrs, 2 hrs, 2.5 hrs, 3 hrs, 4 hrs, 5 hrs, 6 hrs, 7 hrs, 8 hrs, 9 hrs, 10 hrs, 11 hrs, 12 hrs, or 24 hrs, or 48 hours or more from the time of administration.

Regimens of Administration of a Combination of Testosterone, or an Analogue, Derivative Thereof with at Least one ODC Inhibitor

In some embodiments, treatment of a subject with a combination of testosterone, or an analogue, derivative thereof with at least one ODC inhibitor as disclosed herein can be according to the methods as disclosed herein can be therapeutic treatment, e.g., a method of treatment of a low testosterone level in a subject. In some embodiments, therapeutic treatment involves administration of a combination of testosterone, or an analogue, derivative thereof with at least one ODC inhibitor as disclosed herein according to the methods as disclosed herein to a patient suffering from one or more symptoms of or having been diagnosed as being afflicted with a low testosterone level. Relief and even partial relief from one or more of a symptom of low testosterone levels may correspond to an increased sexual drive, increased interest in life or, simply, an increased quality of life. Further, treatments that alleviate a pathological symptom can allow for other treatments to be administered.

In alternative embodiments, the treatment of a subject with a combination of testosterone, or an analogue, derivative thereof with at least one ODC inhibitor as disclosed herein can be according to the methods as disclosed herein can be a prophylactic treatment, for example, to prevent low testosterone levels from occurring in a subject, for example, where the subject is developing age-related low testosterone levels (e.g., undergoing male menopause) or has damage or injury to the testis or Leydig cells, for example, a subject with testicular cancer which is, or has or will undergo testicular cancer treatment, such as for example chemotherapy, radiotherapy, resection or testicular removal and the like. In some embodiments, prophylactic treatments involve administration of a combination of testosterone, or an analogue, derivative thereof with at least one ODC inhibitor as disclosed herein according to a method of the invention to a subject having a been recommended to have, or having undergone a prostate cancer and/or testicular cancer treatment, where it is desirable to prevent the decrease in testosterone levels in the subject as a side-effect of the cancer treatment or testicle removal, and where treatment with testosterone alone is undesirable due to the prostate-associated side effects of testosterone. Administration of a composition comprising a combination of testosterone, or an analogue, derivative thereof with at least one ODC inhibitor as disclosed herein can begin at the beginning or after, or during (e.g., concurrent with) administration of a cancer therapy (e.g., chemotherapy, radiation therapy) etc., and can continue, if necessary, after cancer treatment, and if necessary for the remaining life of the subject. In some embodiments, prophylactic treatment is useful where a subject is likely to be exposed to radiation, for example, subjects who are in or located near an area of a radiation disaster accident, or subjects who are working in a recovery effort in an area that has had a radiation disaster or working in or near a radiation exposure. As demonstrated herein, both prophylactic and therapeutic uses are readily acceptable, because these compounds are generally safe and non-toxic.

With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment or make other alteration to treatment regimen. The dosing schedule can vary from once a week to daily depending on a number of clinical factors, such as the subject's sensitivity to the combination of testosterone, or an analogue, derivative thereof with at least one ODC inhibitor. A desired dose can be administered everyday or every third, fourth, fifth, or sixth day. The desired dose can be administered at one time or divided into subdoses, e.g., 2-4 subdoses and administered over a period of time, e.g., at appropriate intervals through the day or other appropriate schedule. Such sub-doses can be administered as unit dosage forms. In some embodiments of the aspects described herein, administration is chronic, e.g., one or more doses daily over a period of weeks or months. Examples of dosing schedules are administration daily, twice daily, three times daily or four or more times daily over a period of 1 week, 2 weeks, 3 weeks, 4 weeks, 1 month, 2 months, 3 months, 4 months, 5 months, or 6 months or more.

In some embodiments, administration of a combination of testosterone, or an analogue, derivative thereof with at least one ODC inhibitor as disclosed herein can be intermittent; for example, administration can be once every two days, every three days, every five days, once a week, once or twice a month, and the like. The amount, forms, and/or amounts of the different forms of a combination of testosterone, or an analogue, derivative thereof with at least one ODC inhibitor can be varied at different times of administration.

Pulsed administration of a combination of testosterone, or an analogue, derivative thereof with at least one ODC inhibitor as disclosed herein can be used for the treatment of a low testosterone and/or a disorder associated with low testosterone in a subject, e.g., but not limited to obesity, type 2 diabetes, male menopause etc. In some embodiments, pulsed administration of a composition comprising a combination of testosterone, or an analogue, derivative thereof with at least one ODC inhibitor can be used to increase production of testosterone levels in a subject yet reducing prostate-associated side effects (e.g., an increase in prostate growth and/or mass). In alternative embodiments, a pulse of testosterone or an analogue, derivative or salt thereof is given to the subject, with a pulse of at least one ODC inhibitor given to the subject intermittently between the testosterone pulses.

Similarly, pulsed administration of a combination of testosterone, or an analogue, derivative thereof with at least one ODC inhibitor, (or pulses of testosterone or ODC inhibitors alone) can be used for prophylactic treatment, e.g., for example, a subject who will, or has or is currently undergoing chemotherapy and chemoradiation therapy, in particular, where the subject has prostate and/or testicular cancer. In some embodiments, pulsed administration can be more effective than continuous treatment as pulsed doses results in an overall lower amount of compound used than would be expected from continuous administration of the same composition. Each pulse dose can be reduced and the total amount of drug administered over the course of treatment to the patient can be minimized.

With pulse therapy, in vivo levels of testosterone, or an analogue, derivative thereof or the ODC inhibitor as disclosed herein can drop below that level required for effective continuous treatment. Pulsed administration can reduce the amount of a combination of testosterone, or an analogue, derivative thereof with at least one ODC inhibitor as disclosed herein required to be administered to the patient per dose, and/or per total treatment regimen with an increased effectiveness. Pulsed administration can also provide a saving in time, effort and expense and a lower effective dose can lessen the number and severity of complications that can be experienced by a subject. As such, pulsing can be more effective than continuous administration of the same composition.

In some embodiments, individual pulses of a combination of testosterone, or an analogue, derivative thereof with at least one ODC inhibitor, or alternatively pulses of testosterone or an ODC inhibitor alone can be delivered to a subject continuously over a period of several hours, such as about 2, 4, 6, 8, 10, 12, 14 or 16 hours, or several days, such as 2, 3, 4, 5, 6, or 7 days, or from about 1 hour to about 24 hours or from about 3 hours to about 9 hours. Alternatively, periodic doses can be administered in a single bolus or a small number of injections of a combination of testosterone, or an analogue, derivative thereof with at least one ODC inhibitor, or alternatively pulses of testosterone or an ODC inhibitor alone over a short period of time, for example, less than 1 or 2 hours. For example, arginine butyrate can be administered over a period of 4 days with infusions for about 8 hours per day or overnight, followed by a period of 7 days of no treatment.

In some embodiments, an interval between pulses of administration or the interval of no delivery can be greater than 24 hours or can be greater than 48 hours, and can be for even longer such as for 3, 4, 5, 6, 7, 8, 9 or 10 days, two, three or four weeks or even longer. The interval between pulses can be determined by one of ordinary skill in the art, for example, as demonstrated herein in the Examples, by measuring testosterone levels (e.g., total testosterone and/or free testosterone) in the subject after administration of the pulse dose, and administering a pulse when the testosterone level reaches a certain pre-defined low threshold limit. Such pre-defined low threshold limits can be determined by one of ordinary skill in the art, and can be, for example, about less than about 350 ng/dL of total testosterone (TT) levels and/or about 50 pg/ml (or about 15 pg/ml if measured by analogue free test) for free testosterone (FT) level. Alternatively, in some embodiments, the interval between pulses can be calculated by administering another dose of a combination of testosterone, or an analogue, derivative thereof with at least one ODC inhibitor, or alternatively pulses of testosterone or an ODC inhibitor alone, and when the active compound of the composition is no longer detectable in the subject prior to delivery of the next pulse. Alternatively, intervals can also be calculated from the in vivo half-life of the compound present in the composition. For example, the bioavailability of the combination of testosterone, or an analogue, derivative thereof with at least one ODC inhibitor, can be determined at least about 12 hours after administration, or at least about 48 hours after administration. Accordingly, intervals can be calculated as greater than the in vivo half-life, or 2, 3, 4, 5 and even 10 times greater than the functional or composition half-life. Intervals can be 25, 50, 100, 150, 200, 250 300 and even 500 times the half life of the bioavailability a combination of testosterone, or an analogue, derivative thereof and/or the ODC inhibitor.

In some embodiments, the number of pulses in a single therapeutic regimen can be as little as two, but can be from about 5 to 10, 10 to 20, 15 to 30 or more.

In some embodiments, a subject can a combination of testosterone, or an analogue, derivative thereof with at least one ODC inhibitor for life according to the methods of this invention, for example, where the subject has a permanent or incurable low testosterone levels, e.g., where the subject has had damage and/or removal of the prostate and/or one or more testicles, has an inherited genetic low testosterone level, or has age-related low testosterone e.g., male menopause, or a subject who is at risk of prostate cancer and has low testosterone levels. Compositions can be administered by most any means, and can be delivered to the subject as an oral formulation, or injection (e.g. intravenous, subcutaneous, intraarterial), infusion or instillation. Various methods and apparatus for pulsing compositions by infusion or other forms of delivery to the patient are disclosed in U.S. Pat. Nos. 4,747,825; 4,723,958; 4,948,592; 4,965,251 and 5,403,590, which are incorporated herein in their entirety by reference.

In one embodiment, a combination of testosterone, or an analogue, derivative thereof with at least one ODC inhibitor can be administered to a subject for about 2, or about 3, or about 4, or about five days, or more than five days, and then a subsequently administered after an appropriate interval for an additional period of time, for example, for about 2, or about 3, or about 4, or about five days, or more than five days. Cycles of treatment may occur in immediate succession or with an interval of no treatment between cycles.

In some embodiments, a combination of testosterone, or an analogue, derivative thereof with at least one ODC inhibitor can be administered to a subject before development of male menopause or a age-related decrease in testosterone levels, or a chemotherapeutic treatment, or radiation treatment is administered to the subject. In alternative embodiments, a combination of testosterone, or an analogue, or salt or derivative thereof with at least one ODC inhibitor can be co-administered to a subject concurrently with another agent or treatment regimen, e.g., concurrently with a chemotherapeutic treatment, or radiation treatment. In some embodiments, a combination of testosterone, or an analogue, or salt or derivative thereof with at least one ODC inhibitor can also be co-administered with a pharmaceutical composition comprising an comprising one or more addition agents. The pharmaceutical compositions of a combination of testosterone, or an analogue, or salt or derivative thereof with at least one ODC inhibitor can be provided by pulsed administration. For example, a combination of testosterone, or an analogue, or salt or derivative thereof with at least one ODC inhibitor can be administered to a subject, followed by a chemotherapeutic treatment, or radiation treatment after an interval of time has passed, and this order of administration the same or similar time interval can be repeated, for example, at least 1, 2, 3, 4, 5, 6, 7, 8, 9, or 10 or more times.

According to the compositions and methods of the present invention, for the treatment of sexual dysfunction and erectile dysfunction, the testosterone and ODC inhibitor can be administered as the sole active agents or together with another active agent useful in treating sexual dysfunction or erectile dysfunction, such PDE5 inhibitors such as sildenafil (VIAGRA™), vardenafil (LEVITRA™), tadalafil (CIALIST™), avanafil, DA159, dasanatafil, SK350; AGE (advanced glycation end-product) breaker such as alagebrium chloride; alpha 1 blocker such as phentolamine mesylate (VASOMAX™, ROGITINE™); alpha 1A antagonists such as HMP 12; alpha 2 antagonists such as moxisylyte (ERECNOS™), yohimbe; dopamine agonists such as apomorphine, NBI69733; dopamine D4 agonists such as ABT724 and AT670; guanylate cyclase stimulants such as BAY632521; melanocortin agonists such as PT141; oxytocin agonists; FR229934; SCH444877, ATB901, JNJ10258859, prostaglandin agonists such as alprostadil (MUSE™, ALPROX-TD™, VIRIDAL DUO™), radical scavengers such as OX008; rotamase inhibitors such as GPI1485; aviptadil; nitroglycerine; GPCR agonists such as R873; a selective androgen receptor modulator (SARM); or with another 5a-reductase inhibitor.

A combination of testosterone, or an analogue, or salt or derivative thereof with at least one ODC inhibitor can be administrated to a subject in combination with one or more pharmaceutically active agents. Exemplary pharmaceutically active compound include, but are not limited to, those found in Harrison's Principles of Internal Medicine, 13^{th} Edition, Eds. T. R. Harrison et al. McGraw-Hill N.Y., NY; Physicians Desk Reference, 50^{th} Edition, 1997, Oradell N.J., Medical Economics Co.; Pharmacological Basis of Therapeutics, 8th Edition, Goodman and Gilman, 1990; United States Pharmacopeia, The National Formulary, USP XII NF XVII, 1990; current edition of Goodman and Oilman's The Pharmacological Basis of Therapeutics; and current edition of The Merck Index, the complete content of all of which are herein incorporated in its entirety.

### Low Testosterone Disorders and Subjects Amenable to Treatment

In one embodiment, the invention relates to compositions useful in elevating testosterone levels in a subject with low testosterone levels, yet maintaining normal prostate function and avoiding testosterone-induced prostate side effects. In particular as discussed herein, the combination of testosterone, or an analogue, or salt or derivative thereof with at least one ODC inhibitor enables the sexual and anabolic effects of testosterone while preventing the prostate-effects of testosterone. In some embodiments, the combination of testosterone, or an analogue, or salt or derivative thereof with at least one ODC inhibitor as disclosed herein allows the sexual and anabolic effects of testosterone (e.g., muscle growth and increase in muscle mass) while preventing an increase in prostate growth and/or prostate mass by greater than about 40% as compared to the administration of testosterone alone (e.g., in the absence of an ODC inhibitor). In some embodiments, the present invention prevents an increase in prostate growth and/or prostate mass by at least about 10%, or at least about 20%, or at least about 30%, or at least about 40% or more than 40% as compared to the administration of testosterone alone (e.g., in the absence of an ODC inhibitor).

Subjects amenable to treatment are typically male subjects, e.g., male humans, however, female subjects are also be amenable to treatment using the methods and compositions as disclosed herein. In some embodiments, a subject amenable to treatment according to the methods and compositions as disclosed herein has a total testosterone (TT) level of equal or less than about 350-300 ng/dL, or a free testosterone (FT) level of equal or less than about 50 pg/ml or equal or less than about 15 pg/ml (if FT is measured by Analog Free T test). For example, normal levels of total testosterone in males is between about 300-1000 ng/dl, and 15-70 ng/dl or 5-50 ng/dl for premenopausal and post-menopausal females, respectively. Additionally, normal free testosterone (FT) levels in males are between 34-194 pg/ml or about 1-13 pg/ml for females.

In some embodiments, subjects amenable to treatment are subjects currently undergoing or previously undergone exogenous testosterone therapy, e.g., testosterone patches or injections. In some embodiments, the methods and compositions as disclosed herein are useful to wean subjects from testosterone replacement therapy, for example, where due to the exogenous testosterone replacement, the subject's own Leydig cells have reduced the production of endogenous testosterone. For example, subjects undergoing testosterone replacement therapy, e.g., being administered exogenous testosterone, such as athletes taking testosterone for increased sports performance can be administered the compounds as disclosed herein to restart their own testosterone synthesis. Accordingly, in some embodiments, the compounds and methods as disclosed herein can be used by athletes, and can in some embodiments be used alone or in combination with other compounds such as human chrorionic gonaditropin (HCG).

In some embodiments, a subject amenable to treatment according to the methods and compositions as disclosed herein is a hypogonadal subject.

In one embodiment, a subject is a male human over 50 years old. In one class of this embodiment, the subject is a male human over 55 years old. In another class, the subject is a male human with hypogonadism having serum total testosterone less than 350 ng/dL (hypogonadism being defined as a serum total testosterone level less than the lower limit of normal for younger men [LLN], and recognizing that the LLN will be dependent on the laboratory performing the serum testosterone assay). In another class, the subject is a male human with hypogonadism having serum total testosterone less than 300 ng/dL. In another class, the subject is a male human with hypogonadism having serum total testosterone less than 317 ng/dL. In another class, the subject is a male human with hypogonadism having serum total testosterone less than 280 ng/dL. In another class, the subject is a male human with hypogonadism having serum free testosterone less than 7.344 ng/dL (0.255 nmol/L). In another class, the subject is a male human with hypogonadism having serum bioavailable testosterone less than 109.4 ng/dL (3.8 nmol/L). In another embodiment, the subject is a human male with partial androgen deficiency with serum total testosterone less than 400 ng/dL. In another embodiment, the subject is a human male with partial androgen deficiency with serum total testosterone less than 432 ng/dL (15 nmol/L).

In one embodiment, a subject to be treated by the methods and compositions of the present invention is a man with sexual dysfunction. In another embodiment, a subject is a human male with partial androgen deficiency with serum total testosterone<450 ng/dL. In another class of this embodiment, the subject is a male human having a serum total testosterone level<450 ng/dL, as measured by conventional means.

In another class of this embodiment, a subject is a male human having a serum total testosterone level<432 ng/dL. In one class of this embodiment, the subject is a male human having a serum testosterone level<400 ng/dL. In another class of this embodiment, the subject is a male human having a serum total testosterone level<350 ng/dL. In another class of this embodiment, the subject is a male human having a serum total testosterone level<300 ng/dL. In another class of this embodiment, the subject is a male human having a serum total testosterone level<280 ng/dL. Further, in another embodiment, the male subject has a serum total testosterone level of <200 ng/dL.

In another embodiment, a subject does have benign prostatic hyperplasia. In one embodiment, the subject amenable to treatment using the methods as disclosed herein is selected for prostate hyperplasia or a disorder or disease associated with the prostate, e.g., prostate cancer, or have had partial prostate removal for the removal of prostate cancer. In another embodiment, a subject does not have benign prostatic hyperplasia.

In some embodiments, the methods and compositions are used to maintain testosterone levels during the aging process. In some embodiments, the methods and compositions as disclosed herein can be used to for the treatment of hypogonadism and/or male menopause. It seems that there might be a benefit to stimulating hormone production by the patient rather than introducing hormones by a foreign route.

In some embodiments, the methods and compositions as disclosed herein are used to maintain or increase testosterone levels in a subject who has undergone removal of part or a whole testis after testicular cancer, or where the subject has previously, or is undergoing radiation therapy for testicular cancer. In some embodiments, a subject amenable to treatment according to the methods and compositions as disclosed herein is a male or female subject with acquired immune deficiency syndrome (AIDS).

There are correlations between low testosterone levels (e.g., testosterone levels in the lowest quartile) and the development of type 2 diabetes or both metabolic syndrome and diabetes, dyslipidemia, and obesity. Other cross-sectional studies report a correlation between low levels of both free and total testosterone and dyslipidemia, obesity, and insulin resistance or hyperinsulinemia. Thus, the methods and compositions as disclosed herein can be used to elevate the natural production of testosterone in aging men for the treatment of one or more of such low-testosterone associated disease such as, but not limited to; type 2 diabetes, metabolic syndrome, dyslipidemia, obesity, insulin resistance or hyperinsulinemia. In some embodiments, the treatment is preventative treatment, where the subject does not yet have a symptom of the low-testosterone associated disease, but is likely to develop the low-testosterone associated disease based on low levels of free and/or total testosterone. In alternative embodiments, the treatment of a subject encompasses treatment of a symptom of a low-testosterone associated disease, where a subject has one or more symptoms of a low-testosterone associated disease such as, but not limited to; type 2 diabetes, metabolic syndrome, dyslipidemia, obesity, insulin resistance or hyperinsulinemia.

Without wishing to be bound by theory, testosterone (4 androsten 17β-ol-3-one) is a C19 steroid hormone with a molecular weight of 288.4 daltons. Testosterone is the major androgen in males and is controlled by luteinizing hormone (LH). LH is released from the anterior pituitary exerting the primary control on testosterone production, and acting directly on the Leydig cells in the testes, where testosterone is produced. Testosterone stimulates adult maturation of external genitalia and secondary sex organs, and the growth of beard, auxiliary and pubic hair. In addition, testosterone has anabolic effects leading to increased linear growth, nitrogen retention, and muscular development. Clinical evaluation of serum testosterone, along with serum LH, assists in evaluation of hypogonadal males. Major causes of lowered testosterone in males include hypogonadotropic hypogonadism, testicular failure, hyperprolactinemia, hypopituitarism, some types of liver and kidney diseases, and critical illness.

Testosterone levels are much lower in females compared to males. The major sources of testosterone in females are the ovaries, the adrenal glands, and the peripheral conversion of precursors, specifically the conversion of androstenedione to testosterone. In females, the normal levels of androgens may provide a substrate for estrogen production. Increased serum testosterone levels in females may be indicative of polycystic ovary syndrome and adrenal hyperplasia, among other conditions.

Testosterone strongly binds to plasma proteins such as sex hormone-binding globulin (SHBG) or testosterone-estradiol-binding globulin (TEBG). Testosterone also binds with low affinity to CBG (cortisol-binding globulins) and albumin. Less than 2.5% of testosterone circulates unbound to plasma proteins.

Testosterone levels in a subject can be easily measured by ordinary methods commonly known in the art. For example, numerous assays for testosterone are known to those of skill in the art. See, e.g., Marcus and Durnford, Steroids 46: 975-86 (1985); Giraudi et al., Steroids 52: 423-4 (1988); Ooi and Donnelly, Clin. Chem. 44: 2178-82 (1988); Dorgan et al., Steroids 67: 151-8 (2002); Choi et al., Clin. Chem. 49: 322-5 (2003). Additionally, U.S. Patent Application 2008/0166697, which is incorporated herein in its entirety by reference that discloses methods to measure testosterone levels by Mass spectroscopy.

Total Testosterone (TT) is a combination of circulating testosterone bound to carrier proteins (albumin, SHBG, transcortin, transferrin) and the free/unbound hormone. The measurement of TT can be achieved by double isotope techniques and is commonly used for elucidation of difficult clinical diagnoses such as male or female pseudohermaphroditism, congenital adrenal hyperplasia and the androgen insensitivity syndrome. Commercially available kits can readily be used to measure TT, such as commercially available kits from Pantex, DSL, Incostar and the like. Alternatively, serum testosterone levels can be measured by isotope dilution-liquid chromatography (see Bui et al., Ann Clin Biochem 2010; 47:248-252), in the blood, urine or saliva, (commercially available testosterone assay from Salimetrics, LLC, State College, Pa.). The normal range for testosterone levels in men is broad and varies by stage of maturity and age. Decreased levels (hypogonadism) in males may be due to: hypothalamic or pituitary disease, genetic diseases which can cause decreased testosterone production in young men (Klinefelter's, Kallman's, and Prader-Willi syndromes) or testicular failure and infertility (as in myotonic dystrophy, a form of muscular dystrophy), or impaired testosterone production because of acquired damage to the testes, such as alcoholism, physical injury, or viral diseases like mumps.

The optimal levels of testosterone for individual subjects vary due to a huge variance between “normal” levels among men due to age and genetic variation. Levels of total T (TT) are measured in nanograms per deciliter (ng/dL) and can range between 300 ng/dL to 1,000 ng/dL. A level of TT under 350-300 ng/dL is considered a low testosterone level and such subjects are amenable to treatment with the compounds, and methods as disclosed herein.

Free Testosterone (FT) refers to unbound, usable, and beneficial portion of your testosterone. There are three common tests for free testosterone, (i) Calculated Free T, which rather than directly testing for free testosterone, works by running tests for a variety of factors such as SHBG levels, LH levels, and total T. The free T levels are calculated through a formula that eliminates the bound portions, leaving only the free testosterone remaining. (ii) Equilibrium Dialysis is a commonly used standard for testing free T levels, as it is extremely accurate. Great advances in the field have developed rapid, inexpensive equilibrium dialysis equipment that can be used to measure free Testosterone. (iii) Analog Free T test, also referred to as a direct RIA (Radioimmune assay) test can be used to measure FT.

Free Testosterone (FT) is often measured to correct the total testosterone concentration for the effect of variable binding by SHBG. Therefore, FT levels are more appropriate to measure rather than TT when investigating for hypoandrogenicity. Methods available to measure FT can be complex (equilibrium dialysis and calculated free testosterone (CFT)) or simple (the commercial FT kit “Coat-A-Count” using an analog tracer).

FT is measured in pg/ml, and FT levels of under about 50 pg/ml are low, as measured by equilibrium dialysis or calculated free T, and such subjects are amenable to treatment with the compounds, and methods as disclosed herein. In some embodiments, where the Free Testosterone (FT) is measured by an Analog Free T test, a FT level of about 15 pg/mL or lower is considered a low testosterone level, and such subjects are amenable to treatment with the compounds, and methods as disclosed herein.

Bioavaliable (or Bioactive) Testosterone refers to both unbound testosterone, as well as testosterone bound to albumen in the bloodstream, but disregards the unusable testosterone that is already bound to SHBG. This level is frequently used in endocrinological studies, because it acts as a good indicator of hormonal activity in general.

Symptoms of low testosterone include, but are not limited to depression, mental fogginess/fuzziness, difficulty concentrating, anxiety, loss of muscle, increased weight gain, decreased facial hair, decreased sexual desire (e.g., decreased libido), increased erectile dysfunction (ED), decreased concentration and the like.

Andropause (also called “male menopause”) is a normal part of aging; and can be accompanied in some men by a gradual and undesired decline in their sexuality, mood and overall energy. Sometimes it can even expose men to more serious health risks. Andropause, a clinical syndrome similar to the female menopause, can occur in men between the ages of 40 and 80+. Andropause, just like menopause, is characterized by a drop in hormone levels. The bodily changes, as a result of reduced hormones, occur very gradually in men and may be accompanied by adverse changes in attitudes and moods, ongoing fatigue, a loss of vitality, and decreased sex drive. Added to this, there is usually a decline in physical agility and ability. Medical studies have reported that a decreased in testosterone levels or a low testosterone level of below about 350 ng/dl can contribute to the risks for other health problems like heart disease and weak bones.

In healthy men also there is a clear, slow but continuous, age-dependent decline of testosterone (T) levels, which is more pronounced for free T (FT) than for total T, a consequence of the age-associated increase of the levels of sex hormone binding globulin (SHBG); at 75 yr of age mean total T (TT) level in the morning is about two thirds of the mean level at 20-30 yr of age, whereas the mean FT and bioactive T (FT plus albumin bound T) level are only 40% of the mean levels in younger males. Moreover, the circadian rhythm of plasma testosterone levels, with higher levels in the morning than in the evening, is generally lost in elderly men. However, wide inter-individual variations can exist due to genetic factors, body mass index, diet, social habits (alcohol, tobacco), and stress. While andropause is similar to menopause in women, use the andropause is distinct in that males retain their reproductive capacity.

In some embodiments, subjects amenable to treatment are typically male subjects, e.g., male humans, however, female subjects are also be amenable to treatment using the methods and compositions as disclosed herein. In some embodiments, a subject amenable to treatment according to the methods and compositions as disclosed herein has a total testosterone (TT) level of equal or less than about 350-300 ng/dL, or a free testosterone (FT) level of equal or less than about 50 pg/ml or equal or less than about 15 pg/ml (if FT is measured by Analog Free testosterone test). For example, normal levels of total testosterone in males is between about 300-1000 ng/dl, and 15-70 ng/dl or 5-50 ng/dl for premenopausal and post-menopausal females, respectively. Additionally, normal free testosterone (FT) levels in males are between 34-194 pg/ml or about 1-13 pg/ml for females.

In some embodiments, subjects amenable to treatment are subjects currently undergoing or previously undergone exogenous testosterone therapy, e.g., testosterone patches or injections. In some embodiments, the methods and compositions as disclosed herein are useful to wean subjects from testosterone replacement therapy, for example, where due to the exogenous testosterone replacement, the subject's own Leydig cells have reduced the production of endogenous testosterone. For example, subjects undergoing testosterone replacement therapy, e.g., being administered exogenous testosterone, such as athletes taking testosterone for increased sports performance can be administered the compounds as disclosed herein to restart their own testosterone synthesis. Accordingly, in some embodiments, the compounds and methods as disclosed herein can be used by athletes, and can in some embodiments be used alone or in combination with other compounds such as human chrorionic gonaditropin(HCG).

In some embodiments, a subject amenable to treatment according to the methods and compositions as disclosed herein is a hypogonadal subject.

In one embodiment, a subject is a male human over 50 years old. In one class of this embodiment, the subject is a male human over 55 years old. In another class, the subject is a male human with hypogonadism having serum total testosterone less than 350 ng/dL(hypogonadism being defined as serum total testosterone level less than the lower limit of normal for younger men [LLN], and recognizing that the LLN will be dependent on the laboratory performing the serum testosterone assay). In another class, the subject is a male human with hypogonadism having serum total testosterone less than 300 ng/dL. In another class, the subject is a male human with hypogonadism having serum total testosterone less than 317 ng/dL. In another class, the subject is a male human with hypogonadism having serum free testosterone less than 7.344 ng/dL (0.255 nmol/L). In another class, the subject is a male human with hypogonadism having serum bioavailable testosterone less than 109.4 ng/dL (3.8 nmol/L). In another embodiment, the subject is a human male with partial androgen deficiency with serum total testosterone less than 400 ng/dL. In another embodiment, the subject is a human male with partial androgen deficiency with serum total testosterone less than 432 ng/dL (15 nmol/L).

In one embodiment, a subject to be treated by the methods and compositions of the present invention is a man with sexual dysfunction. In another embodiment, a subject is a human male with partial androgen deficiency with serum total testosterone<450 ng/dL. In another class of this embodiment, the subject is a male human having a serum total testosterone level<450 ng/dL, as measured by conventional means.

In another class of this embodiment, a subject is a male human having a serum total testosterone level<432 ng/dL. In one class of this embodiment, the subject is a male human having a serum testosterone level<400 ng/dL. In another class of this embodiment, the subject is a male human having a serum total testosterone level<350 ng/dL. In another class of this embodiment, the subject is a male human having a serum total testosterone level<300 ng/dL. In another class of this embodiment, the subject is a male human having a serum total testosterone level<280 ng/dL. Further, in another embodiment, the male subject has a serum total testosterone level of <200 ng/dL.

In another embodiment, a subject does have benign prostatic hyperplasia. In one embodiment, the subject amenable to treatment using the methods as disclosed herein is selected for prostate hyperplasia or a disorder or disease associated with the prostate, e.g., prostate cancer, or having had partial prostate removal for the removal of prostate cancer. In another embodiment, a subject does not have benign prostatic hyperplasia.

In some embodiments, the methods and compositions are used to maintain testosterone levels during the aging process. In some embodiments, the methods and compositions as disclosed herein can be used to for the treatment to hypogonadism and/or male menopause. It seems that there might be a benefit to stimulating hormone production by the patient rather than introducing hormones by a foreign route.

In some embodiments, the methods and compositions as disclosed herein are used to maintain or increase testosterone levels in a subject who has undergone removal of part or a whole testis after testicular cancer, or where the subject has previously, or is undergoing radiation therapy for testicular cancer. In some embodiments, a subject amenable to treatment according to the methods and compositions as disclosed herein is a male or female subject with acquired immune deficiency syndrome (AIDS).

There are correlations between low testosterone levels (e.g., testosterone levels in the lowest quartile) and the development of type 2 diabetes or both metabolic syndrome and diabetes, dyslipidemia, and obesity. Other cross-sectional studies report a correlation between low levels of both free and total testosterone and dyslipidemia, obesity, and insulin resistance or hyperinsulinemia. Thus, the methods and compositions as disclosed herein can be used to elevate the natural production of testosterone in aging men for the treatment of one or more of such low-testosterone associated disease such as, but not limited to; type diabetes, metabolic syndrome, dyslipidemia, obesity, insulin resistance or hyperinsulinemia. In some embodiments, the treatment is preventative treatment, where the subject does not yet have a symptom of the low-testosterone associated disease, but is likely to develop the low-testosterone associated disease based on low levels of free and/or total testosterone. In alternative embodiments, the treatment of a subject encompasses treatment of a symptom of a low-testosterone associated disease, where a subject has one or more symptoms of a low-testosterone associated disease such as, but not limited to; type 2 diabetes, metabolic syndrome, dyslipidemia, obesity, insulin resistance or hyperinsulinemia.

In some embodiments, a compositions comprising testosterone in combination with an ODC inhibitor as disclosed herein can be used in methods for the treatment of anemia, e.g., of aging subjects and/or subjects with a chronic disease, as well as subjects with sarcopenia and functional limitations associated with chronic disease, such as but not limited to end stage renal disease, chronic obstructive lung disease, congestive heart failure, or other chronic disease and with cancer cachexia.

In other embodiments, a composition comprising a PDE8 inhibitor, and/or a dual PDE8/PDE4 inhibitor compound as disclosed herein can be used in methods and to increase testosterone levels for the treatment of subjects who are XXY males (e.g., have Klinefelter's Syndrome). Without wishing to be bound by theory, XXY boys do not progress normally through puberty, as their testes remain child-sized and therefore do not produce enough of the male hormone testosterone. While adolescent XXY boys are typically taller than average they may lack facial and/or pubic hair. About one-third of XXY boys will develop enlarged breasts, a condition known as gynecomastia, which is different than just fat accumulation that gives the appearance of having breasts—it is true breast development. Accordingly, the methods, and composition comprising a PDE8 inhibitor, and/or a dual PDE8/PDE4 inhibitor compound as disclosed herein can be used to increase testosterone levels in teenage XXY males during puberty and during their adult life.

Damage to the cells of the testis can also cause low testosterone levels, where damage can be a result of, for example, accidents, inflammation of the testicles, testicular cancer, radiation therapy or chemotherapy used to treat testicular cancer. Additionally, diseases that affect the hypothalamus and the pituitary glands can cause low testosterone. These include cancer, inflammation and autoimmune diseases of either gland. Certain drugs can affect the way the pituitary gland works and cause low testosterone, for example, morphine and anabolic steroids. Accordingly, male subjects who have damage to one or both testicles, or have diseases that affect the hypothalamus or pituitary gland, or are taking long term morphine and/or anabolic steroids are amenable to treatment with administration of the compounds as disclosed herein.

Additionally, genetic diseases can result in low testosterone levels in a subject. For example, myotonic dystrophy causes testicular failure between the ages of about 30-40 and thus, such subjects are amenable to treatment according to the methods as disclosed herein using a combination of testosterone, or an analogue, or salt or derivative thereof with at least one ODC inhibitor.

In some embodiments, combination of testosterone, or an analogue, or salt or derivative thereof with at least one ODC inhibitor as disclosed herein can be used for the treatment of a subject whom has, or is undergoing a gender change, for example, from female to male transition.

Accordingly, a combination of testosterone, or an analogue, or salt or derivative thereof with at least one ODC inhibitor can be used for the treatment of low testosterone levels, for example but not limited to, XXY males, damage to the prostate and/or testicles due to cancer and/or cancer treatment, damage or injury to the prostate and/or testicles, including accidents, drug use, anabolic steroid use, and/or low levels due to genetic abnormalities, including but not limited to myotonic dystrophy and the like. There current treatment of low testosterone levels, typically include testosterone replacement therapy (e.g., exogenous supplement of testosterone, administered orally, cutaneous (e.g., in patches) or via subcutaneous or i.p. injection routes), which has limited utility and desirability in treatment of aging men as it often causes adverse side effects associated with the prostate as discussed herein. Additionally, a subject can also experience adverse effects at the site of administration of a testosterone replacement therapy, such as pain and itching at the site of administration. Thus, a combination of testosterone, or an analogue, or salt or derivative thereof with at least one ODC inhibitor as disclosed herein provides substantial advantage over existing low testosterone therapies, in that the testosterone induced side-effects on the prostate are avoided which can occur with administration of testosterone alone.

### Other Uses of the Methods and PDE8 Inhibitors Dual PDE8/PDE4 Inhibitor Compounds

In some embodiments, a combination of testosterone, or an analogue, or salt or derivative thereof with at least one ODC inhibitor can be used for the treatment of autoimmune diseases, or reduce or otherwise prevent multiple sclerosis and other autoimmune diseases associated with chemokine-induced migration of leukocytes. For example, a combination of testosterone, or an analogue, or salt or derivative thereof with at least one ODC inhibitor can be used in an amount that is effective to treat, reduce, alleviate, delay the progression of or otherwise prevent an autoimmune disease or allergic disease selected from the group comprising multiple sclerosis, type 1 diabetes, rheumatoid arthritis, asthma, chronic obstructive pulmonary diseases, inflammatory bowel disease, Alzheimer's disease and other neurodegenerative diseases with inflammatory components, atherosclerosis, vasculitis, and cancer, including metastatic cancer.

In some embodiments, a combination of testosterone, or an analogue, or salt or derivative thereof with at least one ODC inhibitor can be used in a method for increasing testosterone production in a subject, for example a male subject, and/or increasing levels of testosterone in a subject, or in methods for preventing the development of disease or disorder associated with low testosterone, for example, but not limited to, type 2 diabetes, obesity, and the like.

In some embodiments, a composition comprising testosterone in combination with an ODC inhibitor as disclosed herein can be used in methods for the treatment of anemia, e.g., of aging subjects and/or subjects with a chronic disease, as well as subjects with sarcopenia and functional limitations associated with chronic diseases, such as but not limited to, end stage renal disease, chronic obstructive lung disease, congestive heart failure or other chronic disease, and with cancer cachexia.

In some embodiments, a combination of testosterone, or an analogue, or salt or derivative thereof with at least one ODC inhibitor as disclosed herein and/or salts thereof can be used in a method for treating male menopause or low testosterone levels as a complication or side effect of where the subject has been exposed to any one of the following: radiation (e.g., accidental radiation exposure), radiation therapy, chemotherapy, and radiation as a pretreatment to ablate a testicular cancer etc.

In addition to the diagnostic tests described above, clinical features of low testosterone diseases and/or disorders can be monitored for assessment of low testosterone following onset of a low testosterone-associated disease or disorder. These features include, but are not limited to: assessment of the presence of cell damage, assessment of insulin tolerance, obesity, type 2 diabetes etc., and behavioral abnormalities. Such assessment can be done with methods known to one of ordinary skill in the art, such as behavioral testing, blood testing, and imaging studies, such as radiologic studies, CT scans, PET scans, etc.

In some embodiments, a combination of testosterone, or an analogue, or salt or derivative thereof with at least one ODC inhibitor can be used in method to increase or improve well-being in a male subject. In some embodiments, the methods as disclosed herein can be used to treat a subject with a symptom of sedimentary, or lack of well-being.

In alternative embodiments, a combination of testosterone, or an analogue, or salt or derivative thereof with at least one ODC inhibitor can be used in a method for performance enhancement, for example, in to increase athletic performance, or increase stamina and/or muscle strength or muscle size in subjects, including male and female subjects. In such embodiments and other aspects as disclosed herein, the composition of the present invention may be manufactured into liquids, pastes, bars, cakes, powders, granulates, effervescent tablets, tablets, capsules, lozenges, chewing gum, fast melting tablets or wafers, sublingual tablets, a spray or the like, using conventional methods practiced in the food, sweets and pharmaceutical industry. Alternatively, the composition may also be manufactured in the form of or as a part of a food product, such as a liquid, a paste a bar, a cake a powder or a granulate. It may for example be in the form of a fermented food product, a functional food product, or a sport drink or the like as mentioned above. For example an energy bar according to the present invention may include a combination of testosterone, or an analogue, or salt or derivative thereof with at least one ODC inhibitor and can also a variety of other components such as, for example, nuts, crisps, fruit pieces, chocolate, seeds, and the like. Preferred nuts are almonds, peanuts, hazelnuts, cashews, walnuts, pecans, brazil nuts, and the like. Crisp components include rice crisps, corn crisps, oats, wheat flakes, and the like. The chocolate can be any type of chocolate or chocolate like edible component in various forms, such as, for example, chocolate chips, chunks, flakes and the like. Non-limiting examples of seeds include sesame, sun flower, poppy, caraway, fennel and the like. Additionally, traditional food ingredients such as flavors and the like may be included. For example, additional ingredients may include natural and artificial flavors, sweeteners, salt, flavor enhancers, color additives, emulsifiers, stabilizers, fats, preservatives, and the like.

In some embodiments, a combination of testosterone, or an analogue, or salt or derivative thereof with at least one ODC inhibitor can be used in a method for treating sexual dysfunction in men, or increasing sexual performance in a subject, for example, increasing sexual libido, and/or improving semen quality and/or function of sexual anatomy, for example, increased erections in subjects with erectile dysfunction (ED) in male subjects. In some embodiments, the subject is not a human subject, and the subject is a male stud animal for breeding purposes, for example but not limited to, stud animals, both commercial and domestic animals, including but not limited to horses, cattle, dogs and the like.

In some embodiments, a combination of testosterone, or an analogue, or salt or derivative thereof with at least one ODC inhibitor as disclosed herein can be included into animal feeds for mammals. A combination of testosterone, or an analogue, or salt or derivative thereof with at least one ODC inhibitor can be included in said animal feeds the same way it is included in the aforesaid foods and beverages. The animal feeds are not limited to uses for any particular animals. For instance, the animal feeds may be formulated to feed farm animals like cattle and pigs, and companion animals like dogs, cats, and hamsters. The animal feeds may include flour and meat as ingredients. Said flour may be comprised of wheat powder, rice powder, rye powder, oat powder, barley powder, grain powder, corn powder, and soy powder; said flour may also be comprised of two or more of the aforesaid powder. The use of said flour may provide the necessary carbohydrates for the companion animals. Among the aforesaid powder, the wheat powder is preferably used. The wheat powder may be used alone or used with high-grade flour, middle-grade flour, and low-grade flour; the wheat powder may also be used with any other types of flour. The elasticity of the heat-processed animal feeds may be adjusted by combining the wheat powder with wheat grain and soy proteins. After the heat treatment, the lattice-like structures in wheat bran will become enlarged, which helps improve its taste.

### Kits

The invention also provides kits or pharmaceutical packages that include a combination of testosterone, or an analogue, or salt or derivative thereof with at least one ODC inhibitor for use in the prevention and treatment of the diseases and conditions described herein. In addition to a combination of testosterone, or an analogue, or salt or derivative thereof with at least one ODC inhibitor in the form of, for example, tablets, capsules, or lyophilized powders, the kits or packages can include instructions for using a combination of testosterone, or an analogue, or salt or derivative thereof with at least one ODC inhibitor in the prevention or treatment of low testosterone, or diseases and conditions associated with low testosterone levels. A combination of testosterone, or an analogue, or salt or derivative thereof with at least one ODC inhibitor can be provided in the kits or packages in a bottle or another appropriate form (e.g., a blister pack). Optionally, the kits or pharmaceutical packages can also include other pharmaceutically active agents (see, e.g., the agents listed above, such as anti-obesity agents), and/or materials used in administration of the drug(s), such as diluents, needles, syringes, applicators, and the like.

Various embodiments of the disclosure could also include permutations of the various elements recited in the claims as if each dependent claim was a multiple dependent claim incorporating the limitations of each of the preceding dependent claims as well as the independent claims. Such permutations are expressly within the scope of this disclosure.

While the invention has been particularly shown and described with reference to a number of embodiments, it would be understood by those skilled in the art that changes in the form and details may be made to the various embodiments disclosed herein without departing from the spirit and scope of the invention and that the various embodiments disclosed herein are not intended to act as limitations on the scope of the claims. All references cited herein are incorporated in their entirety by reference.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference and may be employed in the practice of the invention. More generally, documents or references are cited in this text, either in a Reference List before the claims, or in the text itself; and, each of these documents or references (“herein cited references”), as well as each document or reference cited in each of the herein cited references (including any manufacturer's specifications, instructions, etc.), is hereby expressly incorporated herein by reference.

The invention can be understood more fully by reference to the following detailed description and illustrative examples, that are intended to exemplify non-limiting embodiments of the invention.

### EXAMPLES

The following examples are provided for illustrative purposes only and are not intended to limit the scope of the invention.

The description of the present invention has been presented for purposes of illustration and description, but is not intended to be exhaustive or limiting of the invention to the form disclosed. The scope of the present invention is limited only by the scope of the following claims. Many modifications and variations will be apparent to those of ordinary skill in the art. The embodiment described and shown in the figures was chosen and described in order to best explain the principles of the invention, the practical application, and to enable others of ordinary skill in the art to understand the invention for various embodiments with various modifications as are suited to the particular use contemplated.

### Example 1

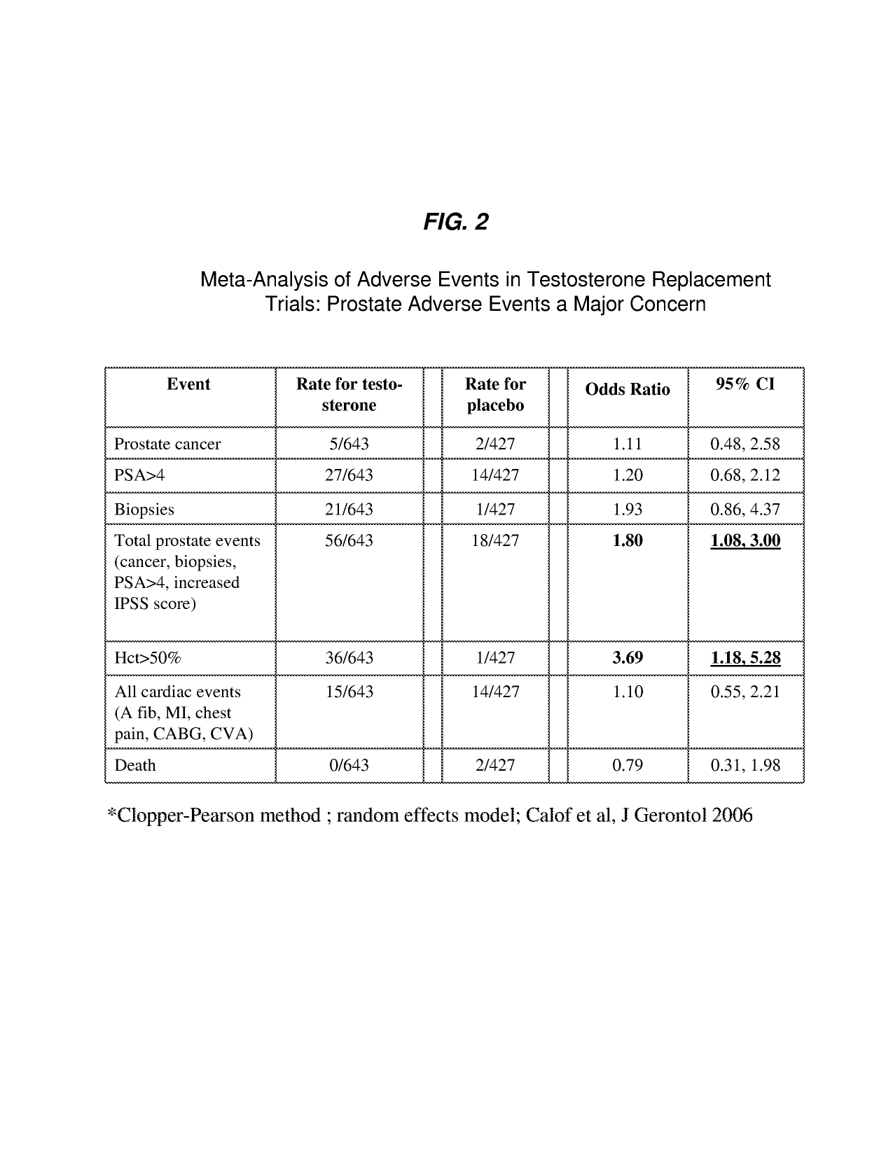

Testosterone increases skeletal muscle mass, muscle strength and physical function, and is a leading candidate as a function promoting therapy for treatment of frailty, sarcopenia, and functional limitations associated with aging and chronic illness. However, concerns about testosterone's adverse effects on the prostate, such as increased prostate growth and/or prostate mass, have limited the enthusiasm for its application as a function promoting anabolic therapy. Examples of adverse-effects on non-anabolic pathways are shown in FIG. 2.

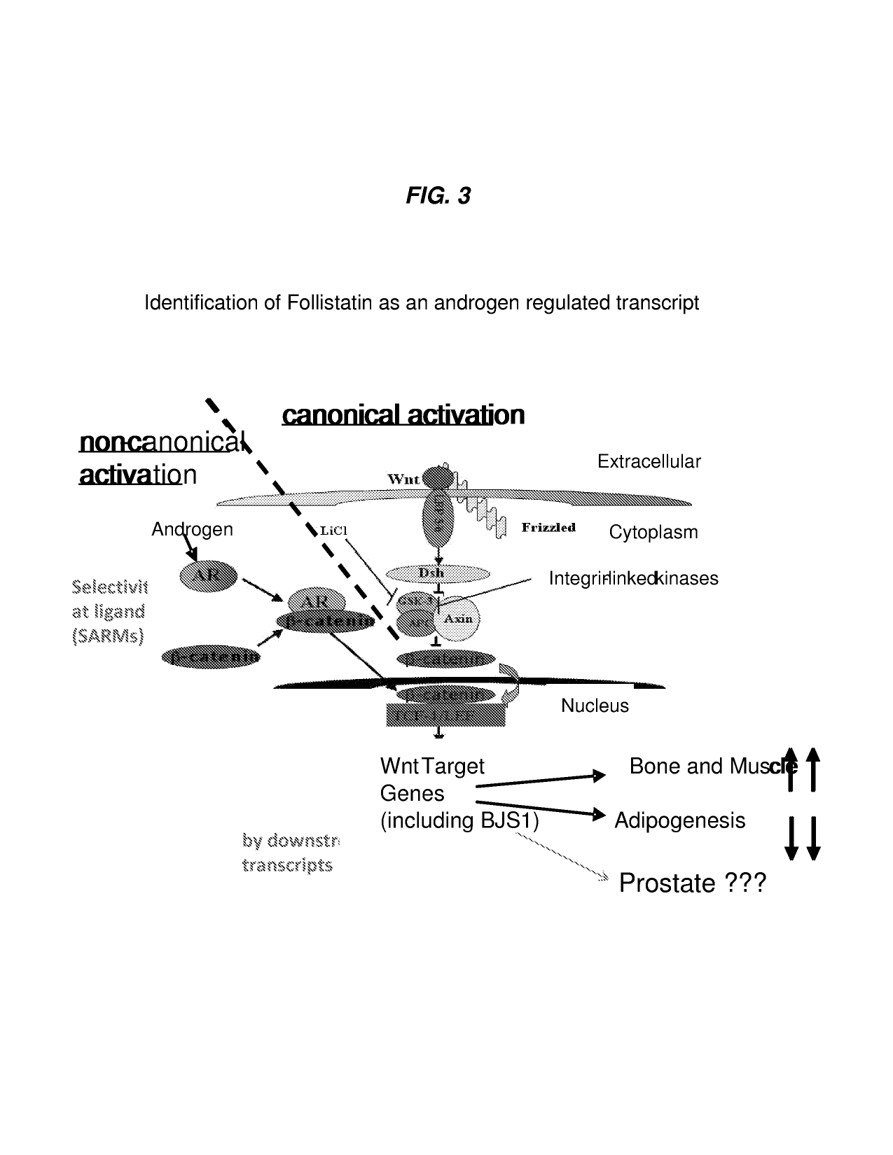

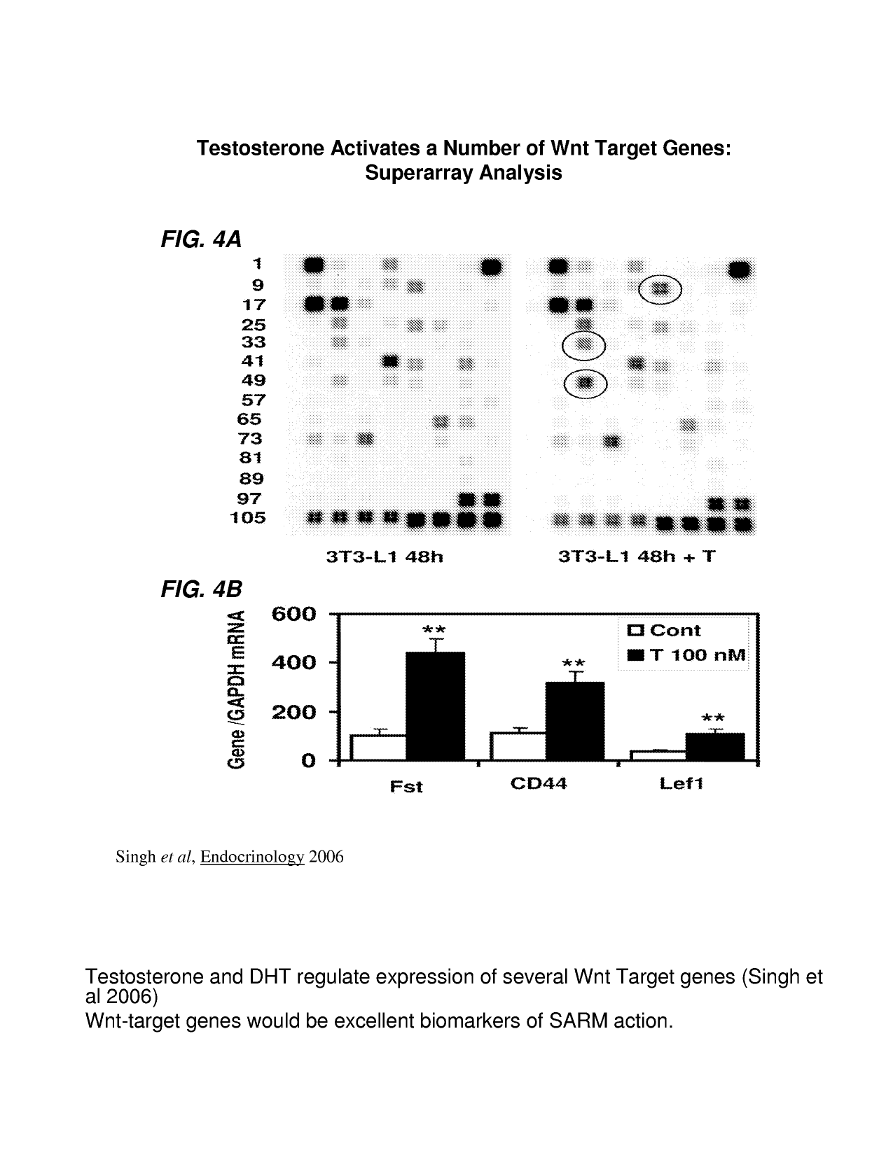

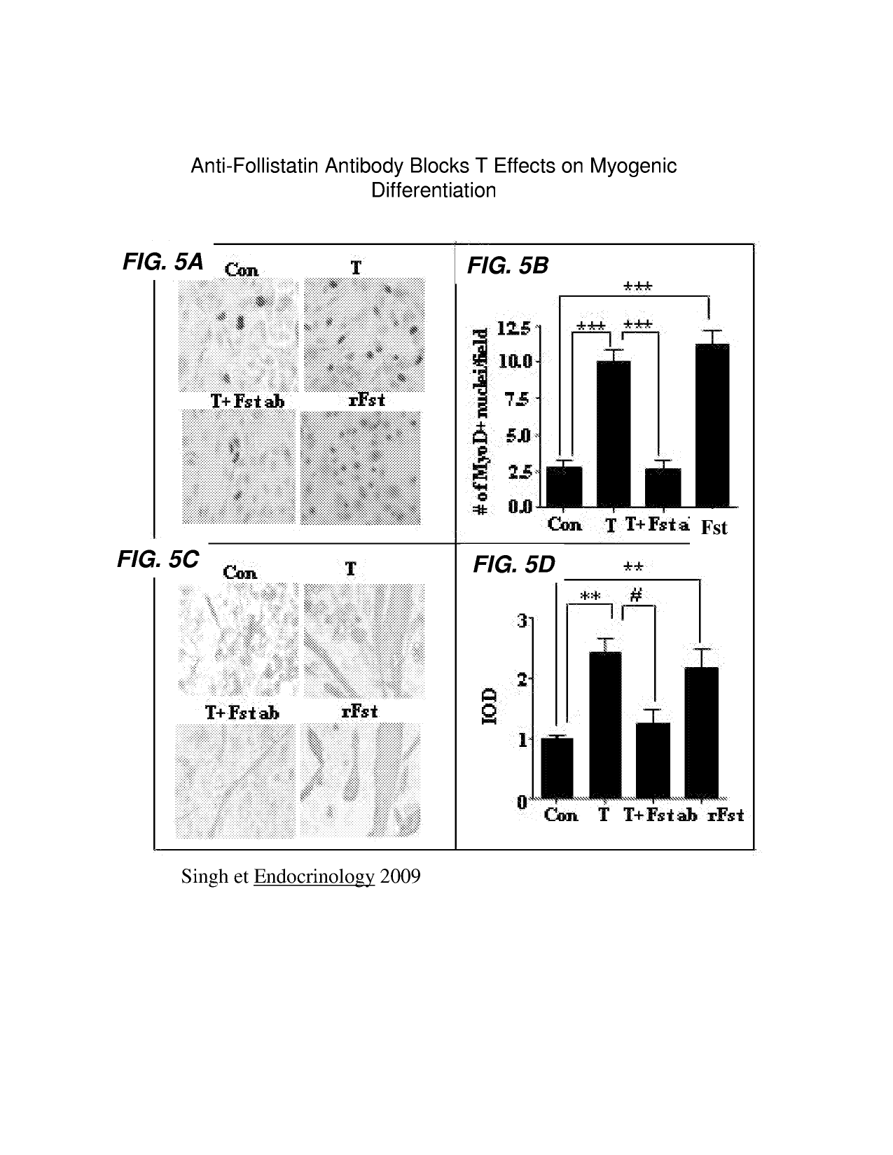

The inventors have demonstrated that testosterone promotes myogenic differentiation of mesenchymal multipotent cells by activating Wnt signaling pathway through β-catenin (See FIGS. 3 and 4A-4B). Follistatin, one of the Wnt-target genes, is essential for mediating testosterone's effects on the skeletal muscle. Knock down of follistatin in vitro blocks testosterone's effects on myogenic differentiation.

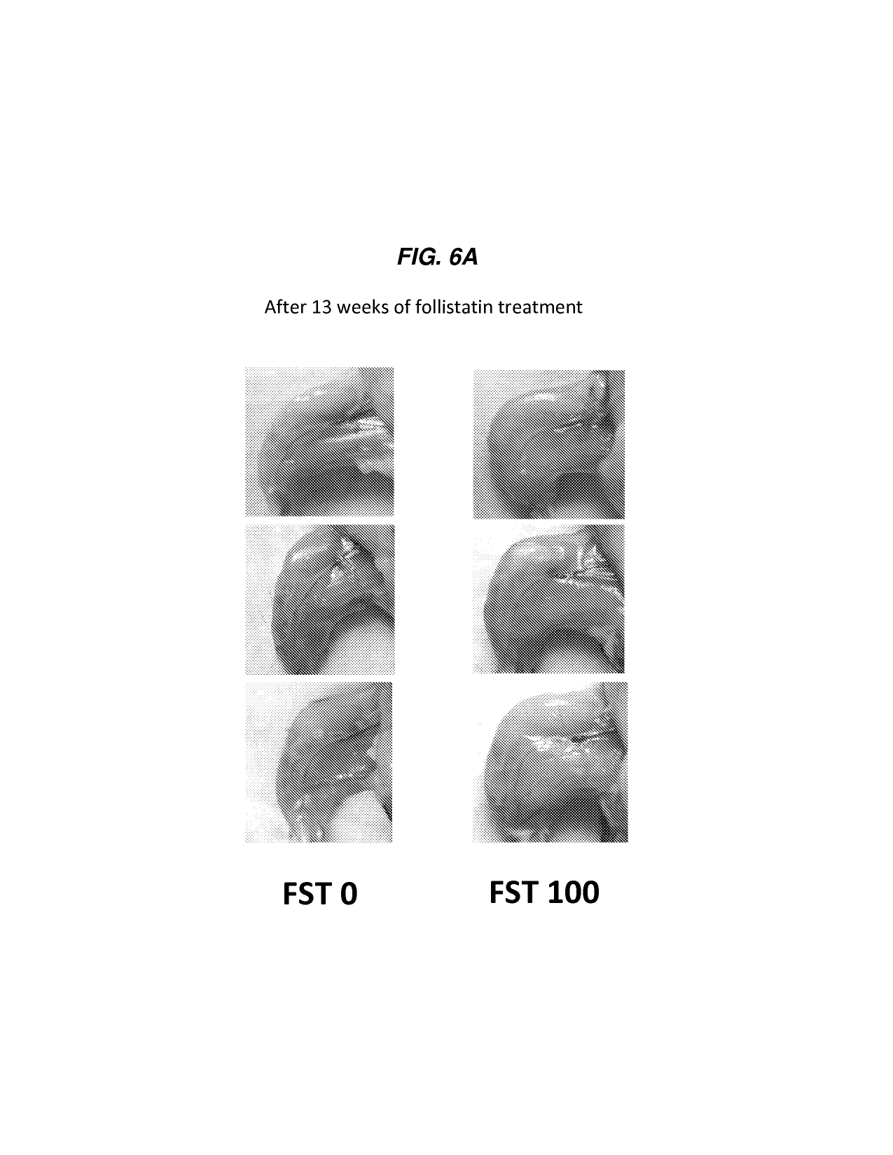

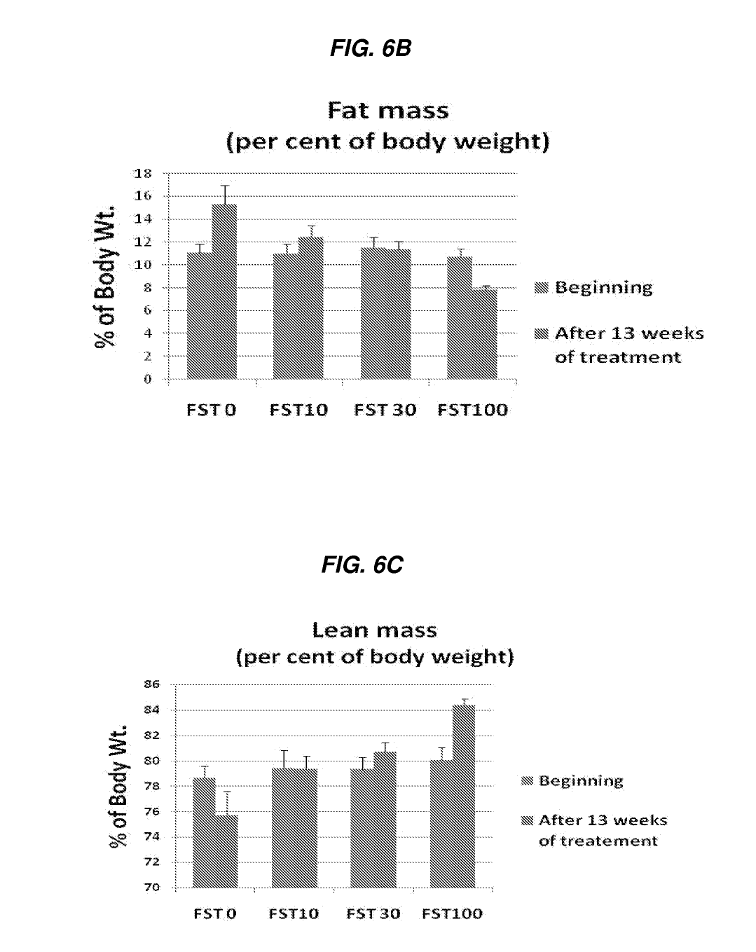

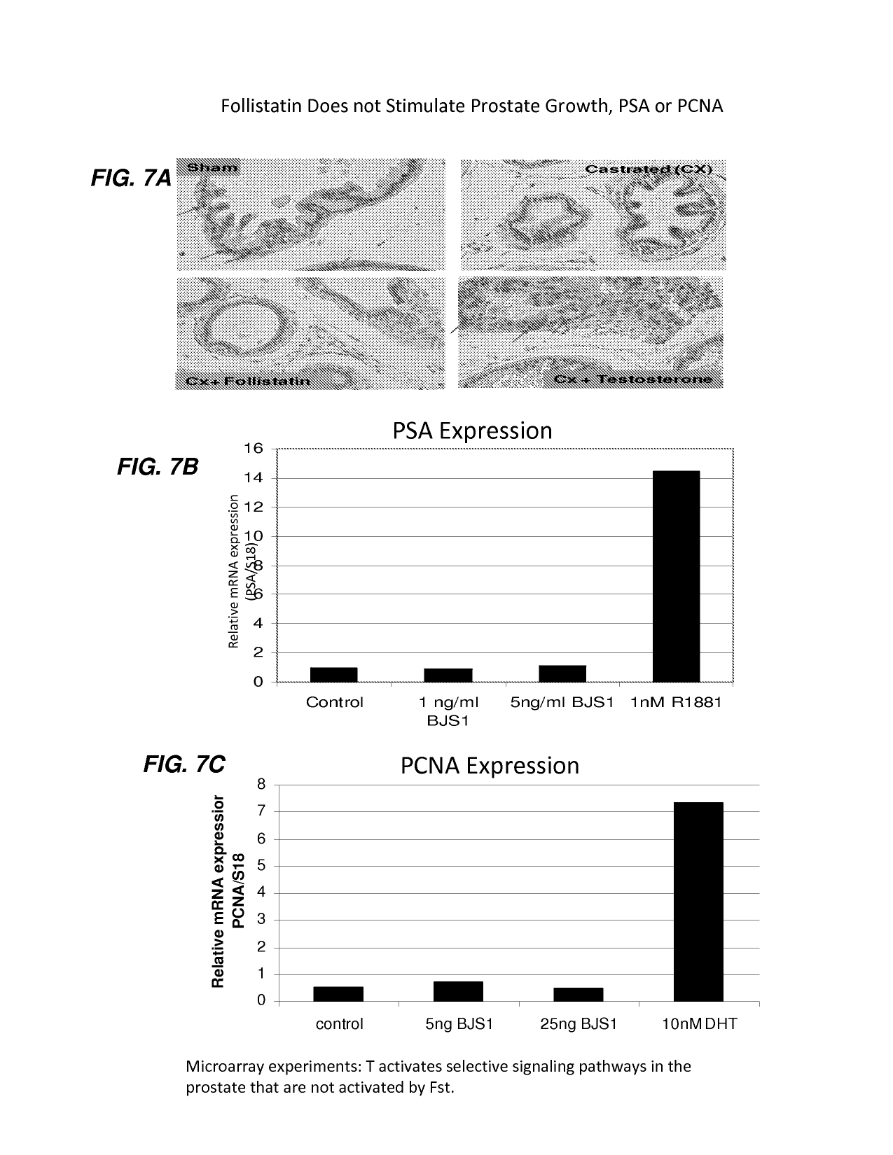

The inventors demonstrated that recombinant follistatin, when administered to castrated mice increases muscle and bone mass, and reduces fat mass (FIG. 6A-6C), but surprisingly, it does not stimulate prostate growth (FIG. 7A). The inventors surprisingly discovered that unlike testosterone, follitatin does not up stimulate the proliferation of androgen-sensitive LNCaP cells in vitro, nor does it up regulate PSA expression (FIG. 7B-7C).

Thus the inventors have demonstrated herein that follistatin, an essential mediator of testosterone's effects on the muscle, selectively increases muscle mass without affecting the prostate. In contrast, testosterone stimulates both muscle mass and prostate growth.

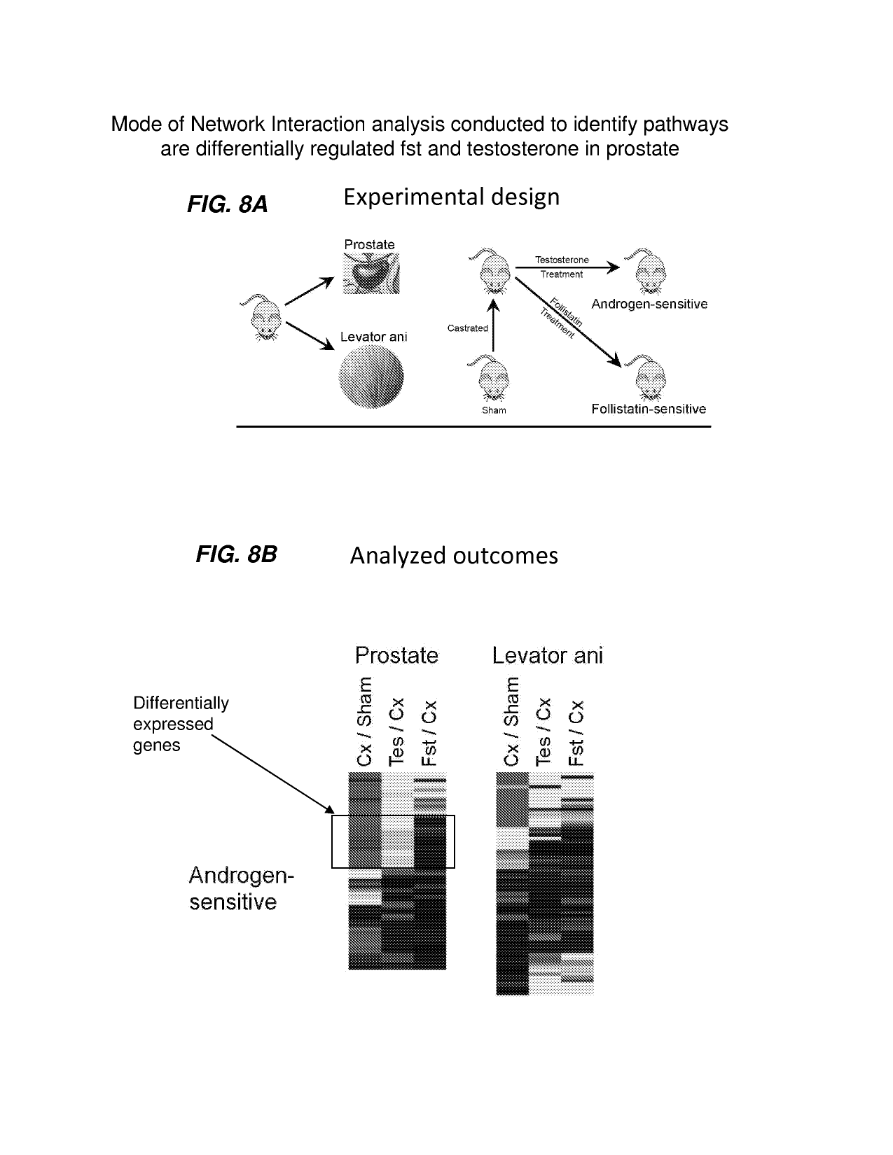

Using microarrays, the inventors elucidated the signaling pathways that are differentially activated in the skeletal muscle and prostate by each of testosterone and follistatin, and assessed the pathways that are specifically activated in the prostate by testosterone, but were not follistatin (FIG. 8A-8B). The inventors determined that testosterone and follstatin both activate common signaling pathways that mediate their anabolic effects on the skeletal muscle, and specifically assessed if testosterone activates one or more unique signaling pathways in the prostate that are not activated by follistatin. The inventors assessed if they could block the pathways that are uniquely activated by testosterone in the prostate, but which are not activated by follistatin, then inhibitors of such pathways in the prostate would allow one to selectively block testosterone's effects on the prostate but not on the muscle.

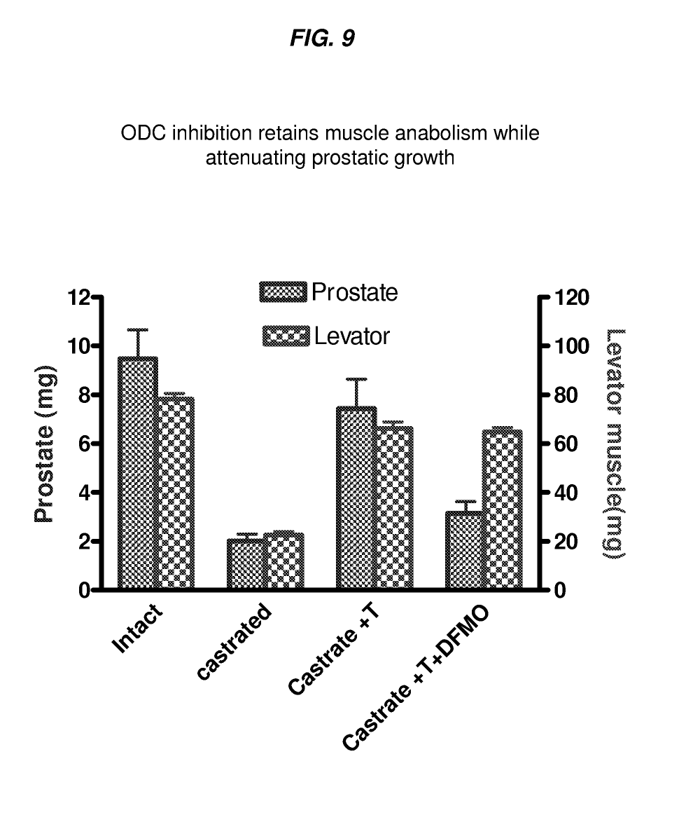

In analysis of microarray data, the inventors discovered ornithine decarboxylase (ODC) and the polyamine pathway as a key signaling pathway that is activated by testosterone in the prostate but not by follistatin. The inventors demonstrated that 2-difluoromethylomithine (DFMO), an inhibitor of the ODC pathway in the prostate blocks testosterone's effects on the prostate, but surprising did not block testosterone's effect on the muscle in mice in vivo (FIG. 9). Thus, the inventor demonstrate that the combined administration of testosterone plus DFMO or other ODC inhibitor provides a unique new approach to achieve selectivity of testosterone's effect without affecting the prostate thus alleviating concerns about its adverse effects on the prostate.

Thus, the inventors have discovered an anabolic drug combination comprising testosterone (or an analogue or pharmaceutically acceptable salt thereof) plus an ODC inhibitor, e.g., DFMO that has sexual and anabolic effects on the skeletal muscle but which, unlike use of testosterone alone, does not affect the prostate. In some embodiments, this anabolic drug combination (testosterone or an analogue or pharmaceutically acceptable salt thereof, plus an ODC inhibitor, e.g., DFMO) would be particularly attractive as a function promoting anabolic therapy or as an androgen replacement therapy in older men with low testosterone levels in whom there is heightened concern about the long term effects of testosterone on the prostate. Also, in men with prostate cancer who are receiving androgen deprivation therapy or who have undergone surgical orchiectomy or who are hypogonadal for other reasons, this drug combination would be ideal because it will allow full androgenic effect on the muscle and other androgen-dependent tissues such as the bone without affecting the prostate. Testosterone is typically contraindicated in these men because there is concern that it can promote growth of prostate cancer.

### REFERENCES

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference and may be employed in the practice of the invention. More generally, documents or references are cited in this text, either in a Reference List before the claims, or in the text itself; and, each of these documents or references (“herein cited references”), as well as each document or reference cited in each of the herein cited references (including any manufacturer's specifications, instructions, etc.), is hereby expressly incorporated herein by reference. Accordingly, the references are each incorporated herein in their entirety by reference.

What is claimed is:

1. A pharmaceutical composition for selectively promoting beneficial effects of testosterone, while preventing side effects related to testosterone administration comprising testosterone or an analogue thereof and an ornithine decarboxylase inhibitor.

2. The pharmaceutical composition as claimed in claim 1, wherein said testosterone analogue is selected from the group consisting of a testosterone ester, a testosterone salt, a testosterone prodrug, a fatty acid ester of testosterone, and a testosterone metabolite.

3. The pharmaceutical composition as claimed in claim 2, wherein said testosterone metabolite is dihydrotestosterone.

4. The pharmaceutical composition as claimed in claim 2, wherein said ester of testosterone is a C2-C13 alkyl ester.

5. The pharmaceutical composition as claimed in claim 4, wherein said C2-C13 alkyl ester is an undecanoate acid ester of testosterone.

6. The pharmaceutical composition as claimed in claim 1, wherein the ornithine decarboxylase inhibitor is selected from the group consisting of 2-difluoromethylornithine, N-(4′-Pyridoxyl)-Ornithine(BOC)-OMe(POB), α-methyl ornithine, 1,4-diamino-2-butanone (DAB), and antizyme (AZ) or a combination thereof.

7. The pharmaceutical composition as claimed in claim 1, wherein said composition is formulated for timed release, sustained release or controlled release of said testosterone or an analogue thereof or said ornithine decarboxylase inhibitor.

8. The pharmaceutical composition as claimed in claim 1, further comprising an agent selected from the group consisting of leutropin, human chorionicgonadotrophin, an anti-cancer agent, and an anti-viral agent.

9. The pharmaceutical composition as claimed in claim 1, wherein said composition is formulated in a gel, tablet, capsule, granulate, food product, troche, dispersion, suspension solution, implant, or patch.

10. A co-administrable product combination comprising a first composition that comprises a testosterone or an analogue thereof, and a second composition that comprises an ornithine decarboxylase (ODC) inhibitor for selectively promoting beneficial effects of testosterone, while preventing side effects related to testosterone administration.

11. The co-administrable product combination as claimed in claim 10, wherein said testosterone analogue is selected from the group consisting of a testosterone ester, a testosterone salt, a testosterone prodrug, a fatty acid ester of testosterone, and a testosterone metabolite.

12. The co-administrable product combination as claimed in claim 11, wherein said testosterone metabolite is dihydrotestosterone.

13. The co-administrable product combination as claimed in claim 11, wherein said ester of testosterone is a C₂-C₁₃ alkyl ester.

14. The co-administrable product combination as claimed in claim 13, wherein said C2-C13 alkyl ester is an undecanoate acid ester of testosterone.

15. The co-administrable product combination as claimed in claim 11, wherein the ornithine decarboxylase inhibitor is selected from the group consisting of 2-difluoromethylornithine, N-(4′-Pyridoxyl)-Ornithine(BOC)-OMe(POB), α-methyl ornithine, 1,4-diamino-2-butanone (DAB), and antizyme (AZ) or a combination thereof.

16. A method for increasing testosterone levels in a subject, for selectively promoting beneficial effects of testosterone, while preventing side effects related to testosterone administration, comprising:
administering testosterone or an analogue thereof to a subject; and
administering an ornithine decarboxylase (ODC) inhibitor to said subject, wherein the administration of said testosterone or analogue thereof and said ornithine decarboxylase (ODC) inhibitor is simultaneous or sequential.

17. The method as claimed in claim 16, wherein the subject has a testosterone level of less than 350 pg/ml.

18. The method as claimed in claim 16, wherein the ornithine decarboxylase inhibitor is selected from the group consisting of 2-difluoromethylornithine, N-(4′-Pyridoxyl)-Ornithine(BOC)-OMe(POB), α-methyl ornithine, 1,4-diamino-2-butanone (DAB), antizyme (AZ) or a combination thereof.

19. The method as claimed in claim 16, wherein the testosterone analogue is selected from the group consisting of a testosterone ester, a testosterone salt, a testosterone prodrug, a fatty acid ester of testosterone, and a testosterone metabolite.

20. The method as claimed in claim 16, wherein said testosterone or analogue thereof and said ODC inhibitor are each administered in an amount of about 0.0002 mg/kg to about 50 mg/kg of body weight per day.
